(19) United States
(12) Patent Application Publication
(10) Pub. No.: US 2019/0231757 A1
(43) Pub. Date: Aug. 1, 2019

(54) COMBINATION OF SPLEEN TYROSINE KINASE INHIBITORS AND OTHER THERAPEUTIC AGENTS

(71) Applicant: Takeda Pharmaceutical Company Limited, Osaka (JP)

(72) Inventors: Rachael L. Brake, Natick, MA (US); Jessica J. Sappal, Wakefield, MA (US); Karuppiah Kannan, Newton, MA (US); Yaping Shou, Boxborough, MA (US)

(21) Appl. No.: 16/317,471

(22) PCT Filed: May 26, 2017
(86) PCT No.: PCT/US2017/034805

§ 371 Date: Jan. 11, 2019

Related U.S. Application Data
(60) Provisional application No. 62/361,999, filed on Jul. 13, 2016.
(60) Provisional application No. 62/425,578, filed on Nov. 22, 2016.

(51) Int. Cl. A61K 31/437; A61K 31/4184; A61K 31/496; A61K 31/519; A61K 31/454; A61K 31/7068; A61K 45/06; C07K 16/28; A61P 35/00

(52) U.S. Cl. CPC A61K 31/437; A61K 31/4184; A61K 31/496; A61K 31/519; A61K 31/454; A61K 9/0053; A61K 45/06; C07K 16/2887; C07K 16/2818; A61P 35/00; A61K 31/7068

(57) ABSTRACT

This disclosure provides combination therapies for treating cancers. In particular, this disclosure provides methods for treating non-Hodgkin lymphoma comprising administering a combination of a SYK inhibitor and a second therapeutic agent.

### CROSS REFERENCE TO RELATED APPLICATION

This application claims priority to U.S. Provisional Application Nos. 62/361,999, filed Jul. 13, 2016 and 62/425,578, filed Nov. 22, 2016, the disclosures of which are incorporated herein by reference in their entireties.

### FIELD

This disclosure provides combination therapies for treating cancers. In particular, this disclosure provides methods for treating non-Hodgkin lymphoma comprising administering a combination of a spleen tyrosine kinase (SYK) inhibitor and a second therapeutic agent.

### BACKGROUND

Spleen tyrosine kinase (SYK) is a 72 kDa non-receptor cytoplasmic tyrosine kinase. SYK has a primary amino acid sequence similar to that of zeta-associated protein-70 (ZAP-70) and is involved in receptor-mediated signal transduction. The N-terminal domain of SYK contains two Src-homology 2 (SH2) domains, which bind to diphosphorylated immunoreceptor tyrosine-based activation motifs (ITAMs) found in the cytoplasmic signaling domains of many immunoreceptor complexes. The C-terminus contains the catalytic domain, and includes several catalytic loop autophosphorylation sites that are responsible for receptor-induced SYK activation and subsequent downstream signal propagation. SYK is expressed in many cell types involved in adaptive and innate immunity, including lymphocytes (B cells, T cells, and NK cells), granulocytes (basophils, neutrophils, and eosinophils), monocytes, macrophages, dendritic cells, and mast cells. SYK is expressed in other cell types, including airway epithelium and fibroblasts in the upper respiratory system. See, e.g., TURNER et al., Immunology Today, 21(3):148-54 (2000); and SANDERSON et al., Inflammation & Allergy—Drug Targets, 8:87-95 (2009).

SYK's role in ITAM-dependent signaling and its expression in many cell types suggest that compounds which inhibit SYK activity may be useful for treating hematological malignancies, such as acute myeloid leukemia, B-cell chronic lymphocytic leukemia, B-cell lymphoma (e.g., mantle cell lymphoma), and T-cell lymphoma (e.g., peripheral T-cell lymphoma); as well as epithelial cancers, such as lung cancer, pancreatic cancer, and colon cancer. See, e.g., HAHN et al., Cancer Cell, 16:281-294 (2009); CHU et al., Immunol. Rev., 165:167-180 (1998); FELDMAN et al., Leukemia, 22:1139-43 (2008); RINALDI et al., Br. J. Haematol., 132:303-316 (2006); STREUBEL et al., Leukemia, 20:313-18 (2006); BUCHNER et al., Cancer Research, 69(13):5424-32 (2009); BAUDOT et al., Oncogene, 28:3261-73 (2009); and SINGH et al., Cancer Cell, 15:489-500 (2009).

It would be beneficial if more effective cancer treatment regimens could be developed. Combinations of cancer treatments that could both treat cancer, and overcome resistance to anticancer agents would be especially helpful. Thus, there is a need for new cancer treatment.

### BRIEF SUMMARY

In certain embodiments, provided herein is a method of treating a non-Hodgkin lymphoma comprising administering to a subject having the non-Hodgkin lymphoma a therapeutically effective amount of a combination comprising a SYK inhibitor and a second therapeutic agent. In certain embodiments, provided herein is a method of treating a non-Hodgkin lymphoma other than chronic lymphocytic leukemia comprising administering to a subject having the non-Hodgkin lymphoma a therapeutically effective amount of a combination comprising a SYK inhibitor and a second therapeutic agent. In certain embodiments, provided herein is a method of treating a non-Hodgkin lymphoma comprising administering to a subject having the non-Hodgkin lymphoma a therapeutically effective amount of a combination comprising a SYK inhibitor and a second therapeutic agent other than ibrutinib, idelalisib, or fludarabine. In certain embodiments, provided herein is a method of treating diffuse large B-cell lymphoma (DLBCL) comprising administering to a subject having DLBCL a therapeutically effective amount of a combination comprising a SYK inhibitor and a second therapeutic agent.

In certain embodiments, the SYK inhibitor for use in the methods and kits provided herein is 6-((1R,2S)-2-aminocyclohexylamino)-7-fluoro-4-(1-methyl-1H-pyrazol-4-yl)-1H-pyrrolo[3,4-c]pyridine-3(2H)-one or the citrate salt thereof (“Compound A”).

In certain embodiments, the second therapeutic agent for use in the methods and kits provided herein is an anticancer agent. In certain embodiments, the second therapeutic agent for use in the methods and kits provided herein is bendamustine, rituximab, gemcitabine, lenalidomide, ibrutinib, venetoclax (ABT-199), nivolumab and/or pembrolizumab.

In certain embodiments, the combination for use in the methods and kits provided herein comprises Compound A and bendamustine. In certain embodiments, the combination for use in the methods and kits provided herein comprises Compound A, bendamustine, and rituximab. In certain embodiments, the combination for use in the methods and kits provided herein comprises Compound A and gemcitabine. In certain embodiments, the combination for use in the methods and kits provided herein comprises Compound A and lenalidomide. In certain embodiments, the combination for use in the methods and kits provided herein comprises Compound A and ibrutinib. In certain embodiments, the combination for use in the methods and kits provided herein comprises Compound A and venetoclax. In certain embodiments, the combination for use in the methods and kits provided herein comprises Compound A and nivolumab. In certain embodiments, the combination for use in the methods and kits provided herein comprises Compound A and pembrolizumab.

In certain embodiments, provided herein is a medical kit for treating a non-Hodgkin lymphoma comprising a therapeutically effective amount of a combination comprising a SYK inhibitor and a second therapeutic agent. In certain embodiments, provided herein is a medical kit for treating a non-Hodgkin lymphoma other than chronic lymphocytic leukemia comprising a therapeutically effective amount of a combination comprising a SYK inhibitor and a second therapeutic agent. In certain embodiments, provided herein is a medical kit for treating a non-Hodgkin lymphoma comprising a therapeutically effective amount of a combination comprising a SYK inhibitor and a second therapeutic agent other than ibrutinib, idelalisib, or fludarabine. In certain embodiments, provided herein is a medical kit for treating diffuse DLBCL comprising a SYK inhibitor and a second therapeutic agent.

### BRIEF DESCRIPTION OF THE DRAWINGS

FIG. 1 exemplifies antitumor activities of Compound A and anti-PD-1 as single agents or in combination against A20 mouse syngeneic B-cell lymphoma.

FIG. 2 exemplifies antitumor activities of Compound A and bendamustine as single agents or in combination against TMD8 DLBCL xenografts.

FIG. 3 exemplifies antitumor activity of Compound A and bendamustine as single agents or in combination against Ly19 xenografts.

FIG. 4 exemplifies antitumor activity of Compound A, ibrutinib, or bendamustine as single agents or in combination against OCI-Ly10 human DLBCL xenografts.

FIG. 5 exemplifies antitumor activity of Compound A, bendamustine, and rituximab as single agents or in combination against OCI-Ly10 human lymphoma xenografts.

FIG. 6 exemplifies antitumor activity of Compound A and gemcitabine as single agents or in combination against OCI-Ly10 xenografts.

FIG. 7 exemplifies antitumor activity of Compound A and gemcitabine as single agents or in combination against TMD8 DLBCL xenografts.

FIG. 8 exemplifies antitumor activity of Compound A and gemcitabine as single agents or in combination against TMD8 DLBCL xenografts.

FIG. 9 exemplifies antitumor activity of Compound A and lenalidomide as single agents or in combination against OCI-Ly10 xenografts.

FIG. 10 exemplifies antitumor activity of Compound A and ABT-199 alone or combined against Ly10 model.

FIG. 11 exemplifies antitumor activity of Compound A and ibrutinib as single agents or in combination against WSU-Luc human lymphoma xenografts.

### DETAILED DESCRIPTION

Unless defined otherwise, all technical and scientific terms used herein have the same meaning as is commonly understood by one of skill in the art to which this disclosure belongs. All patents and publications referred to herein are incorporated by reference in their entirety.

### Certain Terminology

The term “Spleen tyrosine kinase” used herein refers to any member of the Syk family of tyrosine kinases. SYK is a 72 kDa non-receptor cytoplasmic tyrosine kinase.

The term “Spleen tyrosine kinase inhibitor” or “SYK inhibitor” used herein refers to a compound having the ability to interact with Spleen tyrosine kinase and inhibiting its enzymatic activity.

As used herein, the terms “treatment,” “treat,” and “treating” are meant to include the full spectrum of intervention for the cancer from which the subject is suffering, such as administration of the combination to alleviate, slow, stop, or reverse one or more symptoms of the cancer or to delay the progression of the cancer even if the cancer is not actually eliminated. Treatment can include, for example, a decrease in the severity of a symptom, the number of symptoms, or frequency of relapse, e.g., the inhibition of tumor growth, the arrest of tumor growth, or the regression of already existing tumors.

The term “subject”, as used herein, means a mammal, and “mammal” includes, but is not limited to, a human. In certain embodiments, the subject has been treated with an agent, e.g., a SYK inhibitor and/or another agent, prior to initiation of treatment according to the method of the disclosure. In certain embodiments, the subject is at risk of developing or experiencing a recurrence of a cancer. In certain embodiments, the subject is a cancer patient.

The term “anti-cancer agent”, “anti-tumor agent” or “chemotherapeutic agent” refers to any agent useful in the treatment of a neoplastic condition. One class of anti-cancer agents comprises chemotherapeutic agents.

The term “effective amount” or “therapeutically effective amount” refers to that the amount of a compound, or combination of one or more compounds when administered (either sequentially or simultaneously) that elicits the desired biological or medicinal response, e.g., either destroys the target cancer cells or slows or arrests the progression of the cancer in a subject. The therapeutically effective amount may vary depending upon the intended application (in vitro or in vivo), or the subject and disease condition being treated, e.g., the weight and age of the subject, the severity of the disease condition, the manner of administration and the like, which can readily be determined by one skilled in the art. The term also applies to a dose that will induce a particular response in target cells, e.g., reduction of platelet adhesion and/or cell migration. For example, the “therapeutically effective amount” as used herein refers to the amount of a SYK inhibitor and a second therapeutic agent that, when administered in combination has a beneficial effect. In another example, the “therapeutically effective amount” as used herein refers to the amount of a SYK inhibitor, a second therapeutic agent, and an additional therapeutic agent(s) that, when administered in combination has a beneficial effect. In certain embodiments, the combined effect is additive. In certain embodiments, the combined effect is synergistic. Further, it will be recognized by one skilled in the art that in the case of combination therapy, the amount of the SYK inhibitor, the second therapeutic agent, and/or the additional therapeutic agent(s) may be used in a “sub-therapeutic amount”, i.e., less than the therapeutically effective amount of the SYK inhibitor, the second therapeutic agent, or the additional therapeutic agent(s) alone.

The term “about” refers to approximately, in the region of, roughly, or around. When the term “about” is used in conjunction with a number or a numerical range, it means that the number or numerical range referred to is an approximation within experimental variability (or within statistical experimental error), and thus the number or numerical range may vary from, for example, between 1% and 15% of the stated number or numerical range. In general, the term “about” is used herein to modify a numerical value above and below the stated value by a variance of +10%.

The term “combination administration,” or “administered in combination” refers to administering of more than one pharmaceutically active ingredients (including but not limited to a SYK inhibitor, a second therapeutic agent, and one or more additional therapeutic agent(s) as disclosed herein) to a subject. Combination administration may refer to simultaneous administration or may refer to sequential administration of the SYK inhibitor and the second therapeutic agent or the SYK inhibitor, the second therapeutic agent, and the additional therapeutic agent(s) as disclosed herein.

The terms “simultaneous” and “simultaneously” refer to the administration of the SYK inhibitor and the second therapeutic agent as disclosed herein, to a subject at the same time, or at two different time points that are separated by no more than 2 hours. The terms may also refer to the administration of the additional therapeutic agent(s), the SYK inhibitor, and the second therapeutic agent as disclosed herein, to a subject at the same time, or at two different time points that are separated by no more than 2 hours. The terms may also refer to the administration of the additional therapeutic agent(s) and the SYK inhibitor as disclosed herein, to a subject at the same time, or at two different time points that are separated by no more than 2 hours. The terms may also refer to the administration of the additional therapeutic agent(s) and the second therapeutic agent as disclosed herein, to a subject at the same time, or at two different time points that are separated by no more than 2 hours.

The terms “sequential” and “sequentially” refer to the administration of the SYK inhibitor and the second therapeutic agent as disclosed herein, to a subject at two different time points that are separated by more than 2 hours, e.g., about 3 hours, 4 hours, 5 hours, about 8 hours, 12 hours, 1 day, 2 days, 3 days, 4 days, 5 days, 6 days, 7 days or even longer. The terms may also refer to the administration of the SYK inhibitor and the additional therapeutic agent(s) as disclosed herein, to a subject at two different time points that are separated by more than 2 hours, e.g., about 3 hours, 4 hours, 5 hours, about 8 hours, 12 hours, 1 day, 2 days, 3 days, 4 days, 5 days, 6 days, 7 days or even longer. The terms may also refer to the administration of the second therapeutic agent and the additional therapeutic agent(s) as disclosed herein, to a subject at two different time points that are separated by more than 2 hours, e.g., about 3 hours, 4 hours, 5 hours, about 8 hours, 12 hours, 1 day, 2 days, 3 days, 4 days, 5 days, 6 days, 7 days or even longer.

The term “synergistic effect” refers to a situation where the combination of two or more agents produces a greater effect than the sum of the effects of each of the individual agents. The term encompasses not only a reduction in symptoms of the disorder to be treated, but also an improved side effect profile, improved tolerability, improved patient compliance, improved efficacy, or any other improved clinical outcome.

The term a “sub-therapeutic amount” of an agent or therapy is an amount less than the effective amount for that agent or therapy as a single agent, but when combined with an effective or sub-therapeutic amount of another agent or therapy can produce a result desired by the physician, due to, for example, synergy in the resulting efficacious effects, or reduced side effects.

The term “pharmaceutically acceptable salt” refers to salts derived from a variety of organic and inorganic counter ions well known in the art. Pharmaceutically acceptable acid addition salts may be formed with inorganic acids and organic acids. For reviews of suitable salts, see, e.g., BERGE et al, J. Pharm. Sci. 66:1-19 (1977) and Remington: The Science and Practice of Pharmacy, 20th Ed., ed. A. Gennaro, Lippincott Williams & Wilkins, 2000. Non-limiting examples of suitable acid salts includes: hydrochloric acid, hydrobromic acid, sulfuric acid, nitric acid, phosphoric acid, acetic acid, propionic acid, glycolic acid, pyruvic acid, oxalic acid, maleic acid, malonic acid, succinic acid, lactate acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Non-limiting examples of suitable base salts includes: sodium, potassium, lithium, ammonium, calcium, magnesium, iron, zinc, copper, manganese, aluminum, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions of the disclosure is contemplated. Supplementary active ingredients can also be incorporated into the compositions.

The terms “carrier”, “adjuvant”, or “vehicle” are used interchangeably herein, and include any and all solvents, diluents, and other liquid vehicles, dispersion or suspension aids, surface active agents, isotonic agents, thickening or emulsifying agents, preservatives, solid binders, lubricants and the like, as suited to the particular dosage form desired. Remington: The Science and Practice of Pharmacy, 20th Ed., ed. A. Gennaro, Lippincott Williams & Wilkins, 2000 discloses various carriers used in formulating pharmaceutically acceptable compositions and known techniques for the preparation thereof. Except insofar as any conventional carrier medium is incompatible with the compounds of the disclosure, such as by producing any undesirable biological effect or otherwise interacting in a deleterious manner with any other component(s) of the pharmaceutically acceptable composition, its use is contemplated to be within the scope of this disclosure.

Unless otherwise stated, compounds described herein include compounds which differ only in the presence of one or more isotopically enriched atoms. For example, compounds having the present structure except for the replacement of a hydrogen atom by a deuterium or tritium, or the replacement of a carbon atom by a ¹³C- or ¹⁴C-enriched carbon are within the scope of the disclosure.

Unless otherwise stated, compounds described herein include all stereochemical forms of the structure; i.e., the R and S configurations for each asymmetric center. Therefore, single stereochemical isomers as well as enantiomeric and diastereomeric mixtures of the present compounds are within the scope of the disclosure. In the compounds described herein where relative stereochemistry is defined, the diastereomeric purity of such a compound may be at least 80%, at least 90%, at least 95%, or at least 99%. As used herein, the term “diastereomeric purity” refers to the amount of a compound having the depicted relative stereochemistry, expressed as a percentage of the total amount of all diastereomers present.

“Substituted,” when used in connection with a chemical substituent or moiety (e.g., an alkyl group), means that one or more hydrogen atoms of the substituent or moiety have been replaced with one or more non-hydrogen atoms or groups, provided that valence requirements are met and that a chemically stable compound results from the substitution.

The term “alkyl” refers to straight chain and branched saturated hydrocarbon groups, generally having a specified number of carbon atoms (e.g., C₁₋₃ alkyl refers to an alkyl group having 1 to 3 carbon atoms, C₁₋₆ alkyl refers to an alkyl group having 1 to 6 carbon atoms, and so on). Examples of alkyl groups include methyl, ethyl, n-propyl, i-propyl, n-butyl, s-butyl, i-butyl, t-butyl, pent-1-yl, pent-2-yl, pent-3-yl, 3-methylbut-1-yl, 3-methylbut-2-yl, 2-methylbut-2-yl, 2,2,2-trimethyleth-1-yl, n-hexyl, and the like.

“Alkenyl” refers to straight chain and branched hydrocarbon groups having one or more carbon-carbon double bonds, and generally having a specified number of carbon atoms. Examples of alkenyl groups include ethenyl, 1-propen-1-yl, 1-propen-2-yl, 2-propen-1-yl, 1-buten-1-yl, 1-buten-2-yl, 3-buten-1-yl, 3-buten-2-yl, 2-buten-1-yl, 2-buten-2-yl, 2-methyl-1-propen-1-yl, 2-methyl-2-propen-1-yl, 1,3-butadien-1-yl, 1,3-butadien-2-yl, and the like.

“Alkynyl” refers to straight chain or branched hydrocarbon groups having one or more triple carbon-carbon bonds, and generally having a specified number of carbon atoms. Examples of alkynyl groups include ethynyl, 1-propyn-1-yl, 2-propyn-1-yl, 1-butyn-1-yl, 3-butyn-1-yl, 3-butyn-2-yl, 2-butyn-1-yl, and the like.

“Halo,” “halogen” and “halogeno” may be used interchangeably and refer to fluoro, chloro, bromo, and iodo.

“Haloalkyl,” “haloalkenyl,” and “haloalkynyl,” refer, respectively, to alkyl, alkenyl, and alkynyl groups substituted with one or more halogen atoms, where alkyl, alkenyl, and alkynyl are defined above, and generally having a specified number of carbon atoms. Examples of haloalkyl groups include fluoromethyl, difluoromethyl, trifluoromethyl, chloromethyl, dichloromethyl, trichloromethyl, and the like.

“Cycloalkyl” refers to saturated monocyclic and bicyclic hydrocarbon groups, generally having a specified number of carbon atoms that comprise the ring or rings (e.g., C₃₋₈ cycloalkyl refers to a cycloalkyl group having 3 to 8 carbon atoms as ring members). Bicyclic hydrocarbon groups may include isolated rings (two rings sharing no carbon atoms), spiro rings (two rings sharing one carbon atom), fused rings (two rings sharing two carbon atoms and the bond between the two common carbon atoms), and bridged rings (two rings sharing two carbon atoms, but not a common bond). The cycloalkyl group may be attached to a parent group or to a substrate at any ring atom unless such attachment would violate valence requirements. In addition, the cycloalkyl group may include one or more non-hydrogen substituents unless such substitution would violate valence requirements.

Examples of monocyclic cycloalkyl groups include cyclopropyl, cyclobutyl, cyclopentyl, cyclohexyl, and the like. Examples of fused bicyclic cycloalkyl groups include bicyclo[2.1.0]pentanyl (i.e., bicyclo[2.1.0]pentan-1-yl, bicyclo[2.1.0]pentan-2-yl, and bicyclo[2.1.0]pentan-5-yl), bicyclo[3.1.0]hexanyl, bicyclo[3.2.0]heptanyl, bicyclo[4.1.0]heptanyl, bicyclo[3.3.0]octanyl, bicyclo[4.2.0]octanyl, bicyclo[4.3.0]nonanyl, bicyclo[4.4.0]decanyl, and the like. Examples of bridged cycloalkyl groups include bicyclo[2.1.1]hexanyl, bicyclo[2.2.1]heptanyl, bicyclo[3.1.1]heptanyl, bicyclo[2.2.2]octanyl, bicyclo[3.2.1]octanyl, bicyclo[4.1.1]octanyl, bicyclo[3.3.1]nonanyl, bicyclo[4.2.1]nonanyl, bicyclo[3.3.2]decanyl, bicyclo[4.2.2]decanyl, bicyclo[4.3.1]decanyl, bicyclo[3.3.3]undecanyl, bicyclo[4.3.2]undecanyl, bicyclo[4.3.3]dodecanyl, and the like. Examples of spiro cycloalkyl groups include spiro[3.3]heptanyl, spiro[2.4]heptanyl, spiro[3.4]octanyl, spiro[2.5]octanyl, spiro[3.5]nonanyl, and the like. Examples of isolated bicyclic cycloalkyl groups include those derived from bi(cyclobutane), cyclobutanecyclopentane, bi(cyclopentane), cyclobutanecyclohexane, cyclopentanecyclohexane, bi(cyclohexane), etc.

As used herein, “aryl” refers to fully unsaturated monocyclic aromatic hydrocarbons and to polycyclic hydrocarbons having at least one aromatic ring, both monocyclic and polycyclic aryl groups generally having a specified number of carbon atoms that comprise their ring members (e.g., C₆₋₁₄ aryl refers to an aryl group having 6 to 14 carbon atoms as ring members). The aryl group may be attached to a parent group or to a substrate at any ring atom and may include one or more non-hydrogen substituents unless such attachment or substitution would violate valence requirements. Examples of aryl groups include phenyl, biphenyl, cyclobutabenzenyl, indenyl, naphthalenyl, benzocycloheptanyl, biphenylenyl, fluorenyl, groups derived from cycloheptatriene cation, and the like.

“Heterocycle” and “heterocyclyl” may be used interchangeably and refer to saturated or partially unsaturated monocyclic or bicyclic groups having ring atoms composed of carbon atoms and 1 to 4 heteroatoms independently selected from nitrogen, oxygen, and sulfur. Both the monocyclic and bicyclic groups generally have a specified number of carbon atoms in their ring or rings (e.g., C₂₋₅ heterocyclyl refers to a heterocyclyl group having 2 to 5 carbon atoms and 1 to 4 heteroatoms as ring members). As with bicyclic cycloalkyl groups, bicyclic heterocyclyl groups may include isolated rings, spiro rings, fused rings, and bridged rings. The heterocyclyl group may be attached to a parent group or to a substrate at any ring atom and may include one or more non-hydrogen substituents unless such attachment or substitution would violate valence requirements or result in a chemically unstable compound. Examples of monocyclic heterocyclyl groups include oxiranyl, thiaranyl, aziridinyl (e.g., aziridin-1-yl and aziridin-2-yl), oxetanyl, thiatanyl, azetidinyl, tetrahydrofuranyl, tetrahydrothiopheneyl, pyrrolidinyl, tetrahydropyranyl, tetrahydrothiopyranyl, piperidinyl, 1,4-dioxanyl, 1,4-oxathianyl, morpholinyl, 1,4-dithianyl, piperazinyl, 1,4-azathianyl, oxepanyl, thiepanyl, azepanyl, 1,4-dioxepanyl, 1,4-oxathiepanyl, 1,4-oxaazepanyl, 1,4-dithiepanyl, 1,4-thiazepanyl, 1,4-diazepanyl, 3,4-dihydro-2H-pyranyl, 5,6-dihydro-2H-pyranyl, 2H-pyranyl, 1,2,3,4-tetrahydropyridinyl, and 1,2,5,6-tetrahydropyridinyl.

“Heteroaryl” refers to unsaturated monocyclic aromatic groups and to polycyclic groups having at least one aromatic ring, each of the groups having ring atoms composed of carbon atoms and 1 to 4 heteroatoms independently selected from nitrogen, oxygen, and sulfur. Both the monocyclic and polycyclic groups generally have a specified number of carbon atoms as ring members (e.g., C₁₋₉ heteroaryl refers to a heteroaryl group having 1 to 9 carbon atoms and 1 to 4 heteroatoms as ring members) and may include any bicyclic group in which any of the above-listed monocyclic heterocycles are fused to a benzene ring. The heteroaryl group may be attached to a parent group or to a substrate at any ring atom and may include one or more non-hydrogen substituents unless such attachment or substitution would violate valence requirements or result in a chemically unstable compound. Examples of heteroaryl groups include monocyclic groups such as pyrrolyl (e.g., pyrrol-1-yl, pyrrol-2-yl, and pyrrol-3-yl), furanyl, thiopheneyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, and pyrazinyl.

Examples of heteroaryl groups also include bicyclic groups such as benzofuranyl, isobenzofuranyl, benzothiopheneyl, benzo[c]thiopheneyl, indolyl, 3H-indolyl, isoindolyl, 1H-isoindolyl, indolinyl, isoindolinyl, benzimidazolyl, indazolyl, benzotriazolyl, 1H-pyrrolo[2,3-b]pyridinyl, 1H-pyrrolo[2,3-c]pyridinyl, 1H-pyrrolo[3,2-c]pyridinyl, 1H-pyrrolo[3,2-b]pyridinyl, 3H-imidazo[4,5-b]pyridinyl, 3H-imidazo[4,5-c]pyridinyl, 1H-pyrazolo[4,3-b]pyridinyl, 1H-pyrazolo[4,3-c]pyridinyl, 1H-pyrazolo[3,4-c]pyridinyl, 1H-pyrazolo[3,4-b]pyridinyl, 7H-purinyl, indolizinyl, imidazo[1,2-a]pyridinyl, imidazo[1,5-a]pyridinyl, pyrazolo[1,5-a]pyridinyl, pyrrolo[1,2-b]pyridazinyl, imidazo[1,2-c]pyrimidinyl, quinolinyl, isoquinolinyl, cinnolinyl, quinazolinyl, quinoxalinyl, phthalazinyl, 1,6-naphthyridinyl, 1,7-naphthyridinyl, 1,8-naphthyridinyl, 1,5-naphthyridinyl, 2,6-naphthyridinyl, 2,7-naphthyridinyl, pyrido[3,2-d]pyrimidinyl, pyrido[4,3-d]pyrimidinyl, pyrido[3,4-d]pyrimidinyl, pyrido[2,3-d]pyrimidinyl, pyrido[2,3-b]pyrazinyl, pyrido[3,4-b]pyrazinyl, pyrimido[5,4-d]pyrimidinyl, pyrazino[2,3-b]pyrazinyl, and pyrimido[4,5-d]pyrimidinyl.

“Oxo” refers to a double bonded oxygen (═O).

### SYK Inhibitors

In certain embodiments, the SYK inhibitor for use in the methods and kits provided herein is a compound of Formula I,

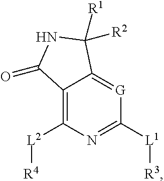

or a pharmaceutically acceptable salt thereof, wherein:

### G is C(R⁵);

L¹ and L² are each independently selected from —NH— and a bond;
R¹ and R² are each independently selected from hydrogen, halo, C₁₋₃ alkyl, and C₁₋₃ haloalkyl, or
R¹ and R², together with the atom to which they are attached, form a C₃₋₆ cycloalkyl;
R³ is selected from C₂₋₆ alkyl, C₃₋₈ cycloalkyl, C₂₋₅ heterocyclyl, and C₁₋₉ heteroaryl, each optionally substituted with from one to five substituents independently selected from halo, oxo, —NO₂, —CN, R⁶, and R⁷;
R⁴ is selected from C₃₋₈ cycloalkyl, C₂₋₅ heterocyclyl, C₆₋₁₄ aryl, and C₁₋₉ heteroaryl, each optionally substituted with from one to five substituents independently selected from halo, oxo, —CN, R⁶, and R⁷;
R⁵ is selected from hydrogen, halo, —CN, C₁₄ alkyl, C₂₋₄ alkenyl, C₂₋₄ alkynyl, C₂₋₅ heterocyclyl, C₁₋₅ heteroaryl, and R¹⁰, wherein the alkyl, alkenyl, alkynyl moieties are each optionally substituted with from one to five substituents independently selected from halo, —CN, oxo, and R¹⁰, and wherein the heterocyclyl moiety has 3 to 6 ring atoms and the heteroaryl moiety has 5 or 6 ring atoms, and the heterocyclyl and heteroaryl moieties are each optionally substituted with from one to four substituents independently selected from halo, —NO₂, —CN, C₁₄ alkyl, C₂₋₄ alkenyl, C₂₋₄ alkynyl, C₁₄ haloalkyl, and R¹⁰;
each R⁶ is independently selected from —OR⁸, —N(R⁸)R⁹, —NR⁸C(O)R⁹, —C(O)R⁸, —C(O)OR⁸, —C(O)N(R⁸)R⁹, —C(O)N(R⁸)OR⁹, —C(O)N(R⁸)S(O)₂R⁹, —N(R⁸)S(O)₂R⁹, —S(O)ₙR⁸, and —S(O)₂N(R⁸)R⁹;
each R⁷ is independently selected from C₁₋₆ alkyl, C₂₋₆ alkenyl, C₂₋₆ alkynyl, C₃₋₆ cycloalkyl-(CH₂)ₘ—, C₆₋₁₄ aryl-(CH₂)ₘ—, C₂₋₅ heterocyclyl-(CH₂)ₘ—, and C₁₋₉ heteroaryl-(CH₂)ₘ—, each optionally substituted with from one to five substituents independently selected from halo, oxo, —NO₂, —CN, C₁₋₆ alkyl, C₁₋₆ haloalkyl, and R¹⁰;
each R⁸ and R⁹ is independently selected from hydrogen or from C₁₋₆ alkyl, C₂₋₆ alkenyl, C₂₋₆ alkynyl, C₃₋₆ cycloalkyl-(CH₂)ₘ—, C₆₋₁₄ aryl-(CH₂)ₘ—, C₂₋₅ heterocyclyl-(CH₂)ₘ—, and C₁₋₉ heteroaryl-(CH₂)ₘ—, each optionally substituted with from one to five substituents independently selected from halo, oxo, —NO₂, —CN, C₁₋₆ alkyl, C₁₋₆ haloalkyl, and R¹⁰;
each R¹⁰ is independently selected
from —OR¹¹, —N(R¹¹)R¹², —N(R¹¹)C(O)R¹², —C(O)R¹¹, —C(O)OR¹¹, —C(O)N(R¹¹)R¹², —C(O)N(R¹¹)OR¹², —C(O)N(R¹¹)S(O)₂R¹², —NR¹¹S(O)₂R¹², —S(O)ₙR¹¹, and —S(O)₂N(R¹¹)R¹²;
each R¹¹ and R¹² is independently selected from hydrogen and C₁₋₆ alkyl;
each n is independently selected from 0, 1 and 2; and
each m is independently selected from 0, 1, 2, 3, and 4;
wherein each of the aforementioned heteroaryl moieties has one to four heteroatoms independently selected from N, O, and S, and each of the aforementioned heterocyclyl moieties is saturated or partially unsaturated and has one or two heteroatoms independently selected from N, O, and S.

In certain embodiments, the SYK inhibitor for use in the methods and kits provided herein is a compound of Formula II, or 6-((1R,2S)-2-aminocyclohexylamino)-7-fluoro-4-(1-methyl-1H-pyrazol-4-yl)-1H-pyrrolo[3,4-c]pyridine-3 (2H)-one:

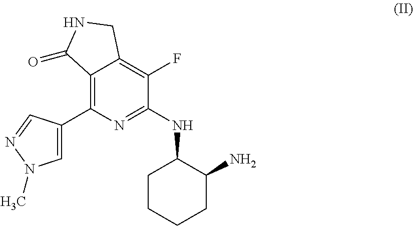 [SMILES: Cn1cc(-c2nc(N[C@@H]3CCCC[C@@H]3N)c(F)c3c2C(=O)NC3)cn1]

or a pharmaceutically acceptable salt thereof.

In certain embodiments, the SYK inhibitor for use in the methods and kits provided herein is a compound of Formula III, or 6-((1R,2S)-2-aminocyclohexylamino)-7-fluoro-4-(1-methyl-1H-pyrazol-4-yl)-1H-pyrrolo[3,4-c]pyridine-3 (2H)-one citrate (“Compound A”):

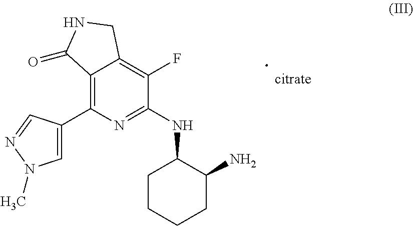 [SMILES: Cn1cc(-c2nc(N[C@@H]3CCCC[C@@H]3N)c(F)c3c2C(=O)NC3)cn1]

or a crystalline form thereof.

The compounds of Formula I, Formula II and Formula III are described in WO 2011/079051, U.S. Pat. No. 8,440,689, and U.S. Ser. No. 14/973,180. They may be prepared by methods known to one skilled in the art and/or according to the methods described in WO 2011/022439, U.S. Pat. No. 8,440,689, and U.S. Ser. No. 14/973,180, each of which is hereby incorporated by reference in its entirety.

Methods of Treatments and/or Medical Uses

In certain embodiments, provided herein is a method of treating a non-Hodgkin lymphoma comprising administering to a subject having the non-Hodgkin lymphoma a therapeutically effective amount of a combination comprising a SYK inhibitor and a second therapeutic agent. In certain embodiments, provided herein is a method of treating a non-Hodgkin lymphoma other than chronic lymphocytic leukemia comprising administering to a subject having the non-Hodgkin lymphoma a therapeutically effective amount of a combination comprising a SYK inhibitor and a second therapeutic agent. In certain embodiments, provided herein is a method of treating a non-Hodgkin lymphoma comprising administering to a subject having the non-Hodgkin lymphoma a therapeutically effective amount of a combination comprising a SYK inhibitor and a second therapeutic agent other than ibrutinib, idelalisib, or fludarabine. In certain embodiments, the non-Hodgkin lymphoma (NHL) is chronic lymphocytic leukemia (CLL), indolent non-Hodgkin lymphoma (iNHL), mantle cell lymphoma (MCL), post-transplant lymphoproliferative disorder (PTLD), or diffuse large B-cell lymphoma (DLBCL). In certain embodiments, the NHL is iNHL, MCL, PTLD, or DLBCL. In certain embodiments the NHL is DLBCL. In certain embodiments, the SYK inhibitor is a compound of Formula I, Formula II, or Formula III. In certain embodiments, the SYK inhibitor is a compound of Formula II or a pharmaceutically acceptable salt thereof. In certain embodiments, the SYK inhibitor is a compound of Formula III or a crystalline form thereof.

In certain embodiments, the combination for methods and kits provided herein further comprises one or more additional therapeutic agent(s).

In certain embodiments, provided herein is a method for treating diffuse large B-cell lymphoma (DLBCL) comprising administering a combination of a SYK inhibitor and a second therapeutic agent. In certain embodiments, the diffuse large B-cell lymphoma is a germinal center B-cell (GCB) DLBCL. In certain embodiments, the diffuse large B-cell lymphoma is a non-germinal center B-cell (non-GCB) DLBCL. In certain embodiments, the diffuse large B-cell lymphoma is an activated B-cell (ABC) DLBCL.

In certain embodiments, the second therapeutic agent is an anticancer agent. In certain embodiments, the second therapeutic agent is bendamustine, rituximab, lenalidomide, ibrutinib, venetoclax, nivolumab and/or pembrolizumab. In certain embodiments, the additional therapeutic agent(s) is an anticancer agent. In certain embodiments, the additional therapeutic agent is rituximab.

In certain embodiments, the second therapeutic agent is a nitrogen mustard. In certain embodiments, provided herein is a method for treating an NHL, comprising administering to a subject having the NHL a therapeutically effective amount of a combination comprising a SYK inhibitor and a nitrogen mustard. In certain embodiments, provided herein is a method of treating an NHL other than CLL comprising administering to a subject having the NHL a therapeutically effective amount of a combination comprising a SYK inhibitor and a nitrogen mustard. In certain embodiments, the NHL is CLL, iNHL, MCL, PTLD, or DLBCL. In certain embodiments, the NHL is iNHL, MCL, PTLD, or DLBCL. In certain embodiments, the NHL is DLBCL. In certain embodiments, provided herein is a method for treating DLBCL, comprising administering to a subject having DLBCL a therapeutically effective amount of a combination comprising a SYK inhibitor and a nitrogen mustard. In certain embodiments, the SYK inhibitor is a compound of Formula I, Formula II, or Formula III. In certain embodiments, the SYK inhibitor is a compound of Formula II or a pharmaceutically acceptable salt thereof. In certain embodiments, the SYK inhibitor is a compound of Formula III or a crystalline form thereof. In certain embodiments, the nitrogen mustard is selected from chlorambucil, uramustine, ifosfamide, melphalan, and bendamustine. In certain embodiments, the nitrogen mustard is bendamustine. In certain embodiments, the SYK inhibitor is a compound of Formula II or a pharmaceutically acceptable salt thereof and the nitrogen mustard is bendamustine. In certain embodiments, the SYK inhibitor is a compound of Formula III or a crystalline form thereof and the nitrogen mustard is bendamustine. In certain embodiments, the combination for methods and kits provided herein further comprises an anti-CD20 antibody. In certain embodiments, the anti-CD20 antibody is selected from rituximab, obinutuzumab, ibritumomab tiuxetan, and tositumomab. In certain embodiments, the anti-CD20 antibody is rituximab. In certain embodiments, the SYK inhibitor is a compound of Formula II or a pharmaceutically acceptable salt thereof, the nitrogen mustard is bendamustine, and the anti-CD20 antibody is rituximab. In certain embodiments, the SYK inhibitor is a compound of Formula III or a crystalline form thereof, the nitrogen mustard is bendamustine, and the anti-CD20 antibody is rituximab. In certain embodiments, provided herein is a method for treating DLBCL comprising administering to a subject having DLBCL a combination comprising a SYK inhibitor of Formula I, Formula II, or Formula III and bendamustine. In certain embodiments, provided herein is a method for treating DLBCL comprising administering to a subject having DLBCL a combination comprising a SYK inhibitor of Formula I, Formula II, or Formula III, bendamustine, and rituximab. In certain embodiments, provided herein is a method for treating DLBCL comprising administering to a subject having DLBCL a combination comprising a SYK inhibitor of Formula II or a pharmaceutically acceptable salt thereof and bendamustine. In certain embodiments, provided herein is a method for treating DLBCL comprising administering to a subject having DLBCL a combination comprising a SYK inhibitor of Formula III or a crystalline form thereof and bendamustine. In certain embodiments, provided herein is a method for treating DLBCL comprising administering to a subject having DLBCL a combination of a SYK inhibitor of Formula II or a pharmaceutically acceptable salt thereof, bendamustine, and rituximab. In certain embodiments, provided herein is a method for treating DLBCL comprising administering to a subject having DLBCL a combination comprising a SYK inhibitor of Formula III or a crystalline form thereof, bendamustine, and rituximab.

In certain embodiments, the second therapeutic agent is a nucleoside analog. In certain embodiments, provided herein is a method for treating an NHL, comprising administering to a subject having the NHL a therapeutically effective amount of a combination comprising a SYK inhibitor and a nucleoside analog. In certain embodiments, provided herein is a method of treating an NHL other than CLL comprising administering to a subject having the NHL a therapeutically effective amount of a combination comprising a SYK inhibitor and a nucleoside analog. In certain embodiments, provided herein is a method for treating an NHL, comprising administering to a subject having the NHL a therapeutically effective amount of a combination of a SYK inhibitor and a nucleoside analog other than fludarabine. In certain embodiments, the NHL is CLL, iNHL, MCL, PTLD, or DLBCL. In certain embodiments, the NHL is iNHL, MCL, PTLD, or DLBCL. In certain embodiments, the NHL is DLBCL. In certain embodiments, provided herein is a method for treating DLBCL, comprising administering to a subject having DLBCL a therapeutically effective amount of a combination comprising a SYK inhibitor and a nucleoside analog. In certain embodiments, the SYK inhibitor is a compound of Formula I, Formula II, or Formula III. In certain embodiments, the SYK inhibitor is a compound of Formula II or a pharmaceutically acceptable salt thereof. In certain embodiments, the SYK inhibitor is a compound of Formula III or a crystalline form thereof. In certain embodiments, the nucleoside analog is selected from gemcitabine and 5-FU. In certain embodiments, the nucleoside analog is gemcitabine. In certain embodiments, the SYK inhibitor is a compound of Formula II or a pharmaceutically acceptable salt thereof and the nucleoside analog is gemcitabine. In certain embodiments, the SYK inhibitor is a compound of Formula III or a crystalline form thereof and the nucleoside analog is gemcitabine. In certain embodiments, provided herein is a method for treating DLBCL comprising administering to a subject having DLBCL a combination comprising a SYK inhibitor of Formula I, Formula II, or Formula III and gemcitabine. In certain embodiments, provided herein is a method for treating DLBCL comprising administering to a subject having DLBCL a combination comprising a SYK inhibitor of Formula II or a pharmaceutically acceptable salt thereof and gemcitabine. In certain embodiments, provided herein is a method for treating DLBCL comprising administering to a subject having DLBCL a combination comprising a SYK inhibitor of Formula III or a crystalline form thereof and gemcitabine.

In certain embodiments, the second therapeutic agent is an immunomodulatory agent. In certain embodiments, provided herein is a method for treating an NHL, comprising administering to a subject having the NHL a therapeutically effective amount of a combination comprising a SYK inhibitor and an immunomodulatory agent. In certain embodiments, provided herein is a method of treating an NHL other than CLL comprising administering to a subject having the NHL a therapeutically effective amount of a combination comprising a SYK inhibitor and immunomodulatory agent. In certain embodiments, the NHL is CLL, iNHL, MCL, PTLD, or DLBCL. In certain embodiments, the NHL is iNHL, MCL, PTLD, or DLBCL. In certain embodiments, the NHL is DLBCL. In certain embodiments, provided herein is a method for treating DLBCL, comprising administering to a subject having DLBCL a therapeutically effective amount of a combination comprising a SYK inhibitor and an immunomodulatory agent. In certain embodiments, an immunomodulatory agent is a thalidomide analogue. In certain embodiments, the thalidomide analogue is lenalidomide. In certain embodiments, provided herein is a method for treating DLBCL, comprising administering to a subject having DLBCL a therapeutically effective amount of a combination comprising a SYK inhibitor and lenalidomide. In certain embodiments, the SYK inhibitor is a compound of Formula I, Formula II, or Formula III. In certain embodiments, the SYK inhibitor is a compound of Formula II or a pharmaceutically acceptable salt thereof. In certain embodiments, the SYK inhibitor is a compound of Formula III or a crystalline form thereof. In certain embodiments, the SYK inhibitor is a compound of Formula II or a pharmaceutically acceptable salt thereof and the thalidomide analogue is lenalidomide. In certain embodiments, the SYK inhibitor is a compound of Formula III or a crystalline form thereof and the thalidomide analogue is lenalidomide. In certain embodiments, provided herein is a method for treating DLBCL, comprising administering to a subject having DLBCL a therapeutically effective amount of a combination comprising a SYK inhibitor of Formula I, Formula II, or Formula III and lenalidomide. In certain embodiments, provided herein is a method for treating DLBCL comprising administering to a subject having DLBCL a combination comprising a SYK inhibitor of Formula II or a pharmaceutically acceptable salt thereof and lenalidomide. In certain embodiments, provided herein is a method for treating DLBCL comprising administering to a subject having DLBCL a combination comprising a SYK inhibitor of Formula III or a crystalline form thereof and lenalidomide.

In certain embodiments, the second therapeutic agent is a BTK inhibitor. In certain embodiments, provided herein is a method for treating an NHL, comprising administering to a subject having the NHL a therapeutically effective amount of a combination comprising a SYK inhibitor and a BTK inhibitor. In certain embodiments, provided herein is a method of treating an NHL other than CLL comprising administering to a subject having the NHL a therapeutically effective amount of a combination comprising a SYK inhibitor and a BTK inhibitor. In certain embodiments, provided herein is a method for treating an NHL, comprising administering to a subject having the NHL a therapeutically effective amount of a combination comprising a SYK inhibitor and a BTK inhibitor other than ibrutinib. In certain embodiments, the NHL is CLL, iNHL, MCL, PTLD, or DLBCL. In certain embodiments, the NHL is iNHL, MCL, PTLD, or DLBCL. In certain embodiments, the NHL is DLBCL. In certain embodiments, provided herein is a method for treating DLBCL, comprising administering to a subject having DLBCL a therapeutically effective amount of a combination comprising a SYK inhibitor and a BTK inhibitor. In certain embodiments, the SYK inhibitor is a compound of Formula I, Formula II, or Formula III. In certain embodiments, the SYK inhibitor is a compound of Formula II or a pharmaceutically acceptable salt thereof. In certain embodiments, the SYK inhibitor is a compound of Formula III or a crystalline form thereof. In certain embodiments, the second therapeutic agent is ibrutinib. In certain embodiments, the BTK inhibitor is ibrutinib. In certain embodiments, the SYK inhibitor is a compound of Formula II or a pharmaceutically acceptable salt thereof and the second therapeutic agent is ibrutinib. In certain embodiments, the SYK inhibitor is a compound of Formula III or a crystalline form thereof and the second therapeutic agent is ibrutinib. In certain embodiments, provided herein is a method for treating DLBCL, comprising administering to a subject having DLBCL a therapeutically effective amount of a combination comprising a SYK inhibitor of Formula I, Formula II, or Formula III and ibrutinib. In certain embodiments, provided herein is a method for treating DLBCL comprising administering to a subject having DLBCL a combination comprising a SYK inhibitor of Formula II or a pharmaceutically acceptable salt thereof and ibrutinib. In certain embodiments, provided herein is a method for treating DLBCL comprising administering to a subject having DLBCL a combination comprising a SYK inhibitor of Formula III or a crystalline form thereof and ibrutinib.

In certain embodiments, the second therapeutic agent is a BCL-2 inhibitor. In certain embodiments, provided herein is a method for treating an NHL, comprising administering to a subject having the NHL a therapeutically effective amount of a combination comprising a SYK inhibitor and a BCL-2 inhibitor. In certain embodiments, provided herein is a method of treating an NHL other than CLL comprising administering to a subject having the NHL a therapeutically effective amount of a combination comprising a SYK inhibitor and a BCL-2 inhibitor. In certain embodiments, the NHL is CLL, iNHL, MCL, PTLD, or DLBCL. In certain embodiments, the NHL is iNHL, MCL, PTLD, or DLBCL. In certain embodiments, the NHL is DLBCL. In certain embodiments, provided herein is a method for treating DLBCL, comprising administering to a subject having DLBCL a therapeutically effective amount of a combination comprising a SYK inhibitor and a BCL-2 inhibitor. In certain embodiments, the SYK inhibitor is a compound of Formula I, Formula II, or Formula III. In certain embodiments, the SYK inhibitor is a compound of Formula II or a pharmaceutically acceptable salt thereof. In certain embodiments, the SYK inhibitor is a compound of Formula III or a crystalline form thereof. In certain embodiments, the BCL-2 inhibitor is venetoclax. In certain embodiments, the SYK inhibitor is a compound of Formula II or a pharmaceutically acceptable salt thereof and the BCL-2 inhibitor is venetoclax. In certain embodiments, the SYK inhibitor is a compound of Formula III or a crystalline form thereof and the BCL-2 inhibitor is venetoclax. In certain embodiments, provided herein is a method for treating DLBCL, comprising administering to a subject having DLBCL a therapeutically effective amount of a combination comprising a SYK inhibitor of Formula I, Formula II, or Formula III and venetoclax. In certain embodiments, provided herein is a method for treating DLBCL comprising administering to a subject having DLBCL a combination comprising a SYK inhibitor of Formula II or a pharmaceutically acceptable salt thereof and venetoclax. In certain embodiments, provided herein is a method for treating DLBCL comprising administering to a subject having DLBCL a combination comprising a SYK inhibitor of Formula III or a crystalline form thereof and venetoclax.

In certain embodiments, the second therapeutic agent is an immunotherapy agent. In certain embodiments, provided herein is a method for treating an NHL, comprising administering to a subject having the NHL a therapeutically effective amount of a combination comprising a SYK inhibitor and an immunotherapy agent. In certain embodiments, provided herein is a method of treating an NHL other than CLL comprising administering to a subject having the NHL a therapeutically effective amount of a combination comprising a SYK inhibitor and an immunotherapy agent. In certain embodiments, the NHL is CLL, iNHL, MCL, PTLD, or DLBCL. In certain embodiments, the NHL is iNHL, MCL, PTLD, or DLBCL. In certain embodiments, the NHL is DLBCL. In certain embodiments, provided herein is a method for treating DLBCL, comprising administering to a subject having DLBCL a therapeutically effective amount of a combination comprising a SYK inhibitor and an immunotherapy agent. In certain embodiments, the SYK inhibitor is a compound of Formula I, Formula II, or Formula III. In certain embodiments, the SYK inhibitor is a compound of Formula II or a pharmaceutically acceptable salt thereof. In certain embodiments, the SYK inhibitor is a compound of Formula III or a crystalline form thereof. In certain embodiments, the immunotherapy agent is an anti-PD-1 or anti-PD-L1 agent, such as an anti-PD-1 or anti-PD-L1 antibody. In certain embodiments, the immunotherapy agent is selected from a PD-1 inhibitor and a PD-L1 inhibitor. In certain embodiments, the immunotherapy agent is a PD-1 inhibitor. In certain embodiments, the immunotherapy agent is a PD-L1 inhibitor. In certain embodiments, the immunotherapy is selected from pembrolizumab and nivolumab. In certain embodiments, the immunotherapy agent is nivolumab. In certain embodiments, the immunotherapy agent is pembrolizumab. In certain embodiments, the SYK inhibitor is a compound of Formula II or a pharmaceutically acceptable salt thereof and the immunotherapy agent is nivolumab. In certain embodiments, the SYK inhibitor is a compound of Formula III or a crystalline form thereof and the immunotherapy agent is nivolumab. In certain embodiments, the SYK inhibitor is a compound of Formula II or a pharmaceutically acceptable salt thereof and the immunotherapy agent is pembrolizumab. In certain embodiments, the SYK inhibitor is a compound of Formula III or a crystalline form thereof and the immunotherapy agent is pembrolizumab. In certain embodiments, provided herein is a method for treating DLBCL, comprising administering to a subject having DLBCL a therapeutically effective amount of a combination comprising a SYK inhibitor of Formula I, Formula II, or Formula III and nivolumab. In certain embodiments, provided herein is a method for treating DLBCL comprising administering to a subject having DLBCL a combination comprising a SYK inhibitor of Formula II or a pharmaceutically acceptable salt thereof and nivolumab. In certain embodiments, provided herein is a method for treating DLBCL comprising administering to a subject having DLBCL a combination comprising a SYK inhibitor of Formula III or a crystalline form thereof and nivolumab. In certain embodiments, provided herein is a method for treating DLBCL, comprising administering to a subject having DLBCL a therapeutically effective amount of a combination comprising a SYK inhibitor of Formula I, Formula II, or Formula III and pembrolizumab. In certain embodiments, provided herein is a method for treating DLBCL comprising administering to a subject having DLBCL a combination comprising a SYK inhibitor of Formula II or a pharmaceutically acceptable salt thereof and pembrolizumab. In certain embodiments, provided herein is a method for treating DLBCL comprising administering to a subject having DLBCL a combination comprising a SYK inhibitor of Formula III or a crystalline form thereof and pembrolizumab.

In certain embodiments, the SYK inhibitor for use in the methods and kits provided herein is administered orally. In certain embodiments, the second therapeutic agent is administered orally or intravenously. In certain embodiments, one or more additional therapeutic agent(s) is administered orally or intravenously. In certain embodiments, the SYK inhibitor for use in the methods and kits provided herein is administered orally and the second therapeutic agent is administered intravenously. In certain embodiments, the SYK inhibitor and the second therapeutic agent are both administered orally. In certain embodiments, the SYK inhibitor is administered orally, the second therapeutic agent is administered intravenously, and the additional therapeutic agent is administered intravenously.

In certain embodiments, the second therapeutic agent and the SYK inhibitor for uses in methods and kits provided herein are administered simultaneously. In certain embodiments, the second therapeutic agent and the SYK inhibitor are administered sequentially. In certain embodiments, the second therapeutic agent is administered prior to the SYK inhibitor. In certain embodiments, the SYK inhibitor is administered prior to the second therapeutic agent. In certain embodiments, the combination for methods and kits provided herein further comprises one or more additional therapeutic agent(s). In certain embodiments, the additional therapeutic agent(s) is administered simultaneously or sequentially with the SYK inhibitor and/or the second therapeutic agent. In certain embodiments, the additional therapeutic agent(s), the SYK inhibitor, and the second therapeutic agent are administered simultaneously. In certain embodiments, the additional therapeutic agent(s) and the SYK inhibitor are administered simultaneously. In certain embodiments, the additional therapeutic agent(s) and the second therapeutic agent are administered simultaneously. In certain embodiments, the additional therapeutic agent(s), the SYK inhibitor, and the second therapeutic agent are administered sequentially. In certain embodiments, the additional therapeutic agent(s) and the SYK inhibitor are administered sequentially. In certain embodiments, the additional therapeutic agent(s) and the second therapeutic agent are administered sequentially. In certain embodiments, the additional therapeutic agent(s) is administered before the SYK inhibitor, after the SYK inhibitor, before the second therapeutic agent, or after the second therapeutic agent. In certain embodiments, the additional therapeutic agent is administered after the second therapeutic agent.

The amounts or suitable doses of the selective inhibitor of SYK, the second therapeutic agent, and the additional therapeutic agent(s) for use in the methods and kits provided herein depends upon a number of factors, including the nature of the severity of the condition to be treated, the particular inhibitor or agent, the route of administration and the age, weight, general health, and response of the individual subject. In certain embodiments, the suitable dose level is one that achieves an effective exposure as measured by increased skin mitotic index, or decreased chromosome alignment and spindle bipolarity in tumor mitotic cells, or other standard measures of effective exposure in cancer patients. In certain embodiments, the suitable dose level is one that achieves a therapeutic response as measured by tumor regression, or other standard measures of disease progression, progression free survival, overall survival, overall response rate (ORR), duration of response (DOR), or time to progression (TTP). In certain embodiments, the suitable dose level is one that achieves a therapeutic response as measured using International Working Group criteria for lymphoma. CHESON et al., J. Clin. Oncol. 25(5):579-86 (2007). In certain embodiments, the suitable dose level is one that achieves this therapeutic response and also minimizes any side effects associated with the administration of the therapeutic agent.

In certain embodiments, the SYK inhibitor for use in the methods and kits provided herein is administered daily. Suitable daily dosages of a SYK inhibitor of Formula I, II, or III can generally range, in single or divided or multiple doses, from about 20 mg to about 200 mg per day, from about 20 mg to about 150 mg per day, or about 40 mg to about 120 mg. In certain embodiments, a dose of the SYK inhibitor is about 20 mg to about 200 mg per day. In certain embodiments, suitable daily doses are about 20 mg, about 30 mg, about 40 mg, 50 mg, about 60 mg, about 70 mg, about 80 mg, about 90 mg, about 100 mg, about 110 mg, about 120 mg, about 130 mg, about 140 mg, about 150 mg per day, about 160 mg per day, about 170 mg per day, about 180 mg per day, about 190 mg per day, or about 200 mg per day. In certain embodiments, the suitable dose may be given once daily or may be divided such that the compound is given twice or three times daily. In certain embodiments, the daily dose of the SYK inhibitor is about 20 mg. In certain embodiments, the daily dose of the SYK inhibitor is about 30 mg. In certain embodiments, the daily dose of the SYK inhibitor is about 40 mg. In certain embodiments, the daily dose of the SYK inhibitor is about 60 mg. In certain embodiments, the daily dose of the SYK inhibitor is about 80 mg. In certain embodiments, the daily dose of the SYK inhibitor is about 100 mg. In certain embodiments, the daily dose of the SYK inhibitor is about 120 mg. In certain embodiments, the daily dose of the SYK inhibitor is about 150 mg. In certain embodiments, the daily dose of the SYK inhibitor is about 200 mg. In certain embodiments, the SYK inhibitor is administered once daily. In certain embodiments, the SYK inhibitor is administered orally, once daily. In certain embodiments, a dose of the SYK inhibitor is about 40 mg per day and the SYK inhibitor is administered once daily. In certain embodiments, a dose of the SYK inhibitor is about 60 mg per day and the SYK inhibitor is administered once daily. In certain embodiments, a dose of the SYK inhibitor is about 80 mg per day and the SYK inhibitor is administered once daily. In certain embodiments, a dose of the SYK inhibitor is about 100 mg per day and the SYK inhibitor is administered once daily.

In certain embodiments, the second therapeutic agent for use in the methods and kits provided herein is administered according to local guidelines. In certain embodiments, the additional therapeutic agent(s) for use in the methods and kits provided herein is administered according to a local guidance. In certain embodiments, the second therapeutic agent is administered according to the product insert or the summary of product characteristic for the second therapeutic agent. In certain embodiments, the additional therapeutic agent is administered according to the product insert or the summary of product characteristic for the additional therapeutic agent.

In certain embodiments, bendamustine is administered according to its product insert or summary of product characteristics. See, e.g., TREANDA (bendamustine hydrochloride) [prescribing information], North Wales, Pa.: Teva Pharmaceuticals USA, Inc., 2015, available at http://www.treandahcp.com/pdf/TREANDA_final_PI.pdf; Bendamustine hydrochloride [summary of product characteristics], East Yorkshire, UK: Dr. Reddy's Laboratories (UK) Ltd., 2015, available at http://www.mhra.gov.uk/home/groups/spcpil/documents/spcpil/con1450417269771.pdf. In certain embodiments, rituximab is administered according to its product insert or summary of product characteristics. See, e.g., RITUXAN (rituximab) [prescribing information], San Francisco, Calif.: Genentech, Inc., 2013, available at http://www.accessdata.fda.gov/drugsatfda_docs/label/2013/103705s54141bl.pdf; MabThera 100 mg concentrate for solution for infusion (rituximab) [summary of product characteristics], Germany: Roche Pharma AG, 2015. In certain embodiments, gemcitabine is administered according to its product insert or summary of product characteristics. See, e.g., GEMCITABINE (gemcitabine hydrochloride) [prescribing information], Lake Forest, Ill.: Zydus Hospira Oncology Private Ltd., 2014, available at https://www.hospira.com/en/images/EN-3523_tcm81-92678.pdf; GEMZAR [summary of product characteristics], Hampshire, UK: Eli Lilly and Company Limited, 2014, available at http://www.mhra.gov.uk/home/groups/spcpil/documents/spcpil/con1463720303037.pdf. In certain embodiments, lenalidomide is administered according to its product insert or summary of product characteristics. See, e.g., REVLIMID (lenalidomide) [prescribing information], Summit, N.J.: Celgene Corporation, 2015, available at http://www.revlimid.com/wp-content/uploads/full-prescribing-information.pdf, Revlimid 2.5 mg hard capsules (lenalidomide) [summary of product characteristics], United Kingdom: Penn Pharmaceutical Services Limited, 2015. In certain embodiments, ibrutinib is administered according to its product insert or summary of product characteristics. See, e.g., IMBRUVICA (ibrutinib) [prescribing information], Pharmacylics LLC, 2016, available at https://www.imbruvica.com/docs/librariesprovider7/default-document-library/prescribing_information.pdf; IMBRUVICA 140 mg hard capsules (ibrutinib) [summary of product characteristics], Belgium: Janssen Pharmaceutica Nev., 2015. In certain embodiments, venetoclax is administered according to its product insert or summary of product characteristics. See, e.g., VENCLEXTA (venetoclax) [prescribing information], North Chicago, Ill., AbbVie Inc., 2016, available at http://www.rxabbvie.com/pdf/venclexta.pdf. In certain embodiments, nivolumab is administered according to its U.S. product insert or summary of product characteristics. See, e.g., OPDIVO (nivolumab) [prescribing information], Princeton, N.J.: Bristol-Myers Squibb, 2016, available at http://packageinserts.bms.com/pi/pi_opdivo.pdf; OPDIVO 10 mg/mL concentrate for solution for infusion (nivolumab) [summary of product characteristics], available at http://www.ema.europa.eu/docs/en_GB/document_library/EPAR_-_Product_Information/human/003985/WC500189765.pdf.

In certain embodiments, bendamustine is bendamustine hydrochloride. In certain embodiments, bendamustine hydrochloride is bendamustine hydrochloride monohydrate. The term bendamustine includes bendamustine chloride and bendamustine hydrochloride monohydrate. The term bendamustine chloride includes bendamustine hydrochloride monohydrate. In certain embodiments, bendamustine is administered on days 1 and 2 of a 21 day cycle at about 90 mg/m² dose. In certain embodiments, bendamustine is administered up to 8 cycles. In certain embodiments, bendamustine administered intravenously. In certain embodiments, bendamustine is administered over 60 minutes. In certain embodiments, bendamustine administered intravenously over 60 minutes on days 1 and 2 of a 21 day cycle at about 90 mg/m² dose up to 8 cycles.

In certain embodiments, bendamustine is in a form of bendamustine hydrochloride, a solution or a lyophilized powder. In certain embodiments, bendamustine is in a form of bendamustine hydrochloride, a solution of 45 mg/0.5 mL or 180 mg/2 mL in a single-dose vial. In certain embodiments, bendamustine is in a form of bendamustine hydrochloride, 25 mg or 100 mg lyophilized powder in a single-dose vial for reconstitution. In certain embodiments, bendamustine is in a form of bendamustine hydrochloride monohydrate, 25 mg (1 vial) or 100 mg (1 vial) powder for concentrate for solution for infusion.

In certain embodiments, bendamustine is infused at 100 mg/m² dose intravenously over 30 minutes on Days 1 and 2 of a 28-day cycle, up to 6 cycles. In certain embodiments, bendamustine dose is modified for hematologic toxicity: for Grade 3 or greater toxicity, dose is reduced to 50 mg/m² on Days 1 and 2; if Grade 3 or greater toxicity recurs, dose is reduced to 25 mg/m² on Days 1 and 2. In certain embodiments, bendamustine dose is modified for non-hematologic toxicity: for clinically significant Grade 3 or greater toxicity, dose is reduced to 50 mg/m² on Days 1 and 2 of each cycle. In certain embodiments, bendamustine dose re-escalation may be considered. In certain embodiments, bendamustine is administered at 50 mg/m² dose. In certain embodiments, bendamustine is administered at 25 mg/m² dose. In certain embodiments, bendamustine is infused at 120 mg/m² dose intravenously over 60 minutes on Days 1 and 2 of a 21-day cycle, up to 8 cycles. In certain embodiments, bendamustine dose is modified for hematologic toxicity: for Grade 4 toxicity, the dose is reduced to 90 mg/m² on Days 1 and 2 of each cycle; if Grade 4 toxicity recurs, the dose is reduced to 60 mg/m² on Days 1 and 2 of each cycle. In certain embodiments, bendamustine dose is modified for non-hematologic toxicity: for Grade 3 or greater toxicity, the dose is reduced to 90 mg/m² on Days 1 and 2 of each cycle; if Grade 3 or greater toxicity recurs, the dose is reduced to 60 mg/m² on Days 1 and 2 of each cycle. In certain embodiments, treatment is delayed for Grade 4 hematologic toxicity or clinically significant ≥Grade 2 non-hematologic toxicity. In certain embodiments, bendamustine is administered at 90 mg/m² dose. In certain embodiments, bendamustine is administered at 60 mg/m² dose.

In certain embodiments, bendamustine is administered as intravenous infusion over 30-60 minutes. In certain embodiments, bendamustine is administered at a 100 mg/m² body surface area dose on days 1 and 2; every 4 weeks. In certain embodiments, bendamustine is administered at 120 mg/m² body surface area dose on days 1 and 2, every 3 weeks. In certain embodiments, bendamustine is administered at 120-150 mg/m² body surface area dose on days 1 and 2, every 4 weeks. In certain embodiments, treatment is terminated or delayed if leukocyte and/or platelet values have dropped to <3,000/μl or <75,000/μl, respectively; treatment can be continued after leukocyte values have increased to >4,000/μl and platelet values to >100,000/μl. In certain embodiments, the leukocyte and platelet Nadir is reached after 14-20 days with regeneration after 35 weeks; during therapy free intervals strict monitoring of the blood count is recommended. In certain embodiments, in case of nonhaematological toxicity dose reductions are be based on the worst Common Toxicity Criteria (CTC) grades in the preceding cycle: a 50% dose reduction is recommended in case of CTC grade 3 toxicity; an interruption of treatment is recommended in case of CTC grade 4 toxicity. In certain embodiments, bendamustine dose is reduced by 50%. In certain embodiments, if a patient requires a dose modification the individually calculated reduced dose must be given on day 1 and 2 of the respective treatment cycle. In certain embodiments, a 30% dose reduction of bendamustine is recommended in patients with moderate hepatic impairment (serum bilirubin 1.2-3.0 mg/dl). In certain embodiments, bendamustine dose is reduced by 30%.

In certain embodiments, rituximab is administered on day 1 of a 21 day cycle at about 375 mg/m² dose. In certain embodiments, rituximab is administered up to 8 cycles. In certain embodiments, rituximab is administered intravenously. In certain embodiments, rituximab is administered per local guidelines. In certain embodiments, rituximab is administered intravenously per local guidelines on day 1 of a 21 day cycle at about 375 mg/m² dose up to 8 cycles. In certain embodiments, bendamustine is administered on days 1 and 2 of a 21 day cycle at about 90 mg/m² dose and rituximab is administered on day 1 of a 21 day cycle at about 375 mg/m² dose. In certain embodiments, bendamustine administered intravenously over 60 minutes on days 1 and 2 of a 21 day cycle at about 90 mg/m² dose up to 8 cycles and rituximab is administered intravenously per local guidelines on day 1 of a 21 day cycle at about 375 mg/m² dose up to 8 cycles.

In certain embodiments, rituximab is in a form of 100 mg/10 mL or 500 mg/50 mL solution in a single-use vial. In certain embodiments, rituximab is in a form of 1400 mg/11.7 mL (1 vial) or 1600 mg/13.4 mL (1 vial) solution for subcutaneous injection.

In certain embodiments, rituximab is administered as an intravenous infusion at 375 mg/m² dose. In certain embodiments, rituximab is administered as an intravenous infusion at 375 mg/m² dose once weekly for 4 or 8 doses. In certain embodiments, rituximab is administered as an intravenous infusion at 375 mg/m² dose once weekly for 4 doses. In certain embodiments, rituximab is administered as an intravenous infusion at 375 mg/m² dose on Day 1 of each cycle of chemotherapy, for up to 8 doses. In certain embodiments, rituximab is administered for eight weeks following the completion of rituximab administration in a combination therapy. In certain embodiments, rituximab is administered every 8 weeks for 12 doses. In certain embodiments, following completion of 6-8 cycles of chemotherapy, rituximab is administered once weekly for 4 doses at 6-month intervals to a maximum of 16 doses. In certain embodiments, rituximab is administered on Day 1 of each cycle of chemotherapy for up to 8 infusions. In certain embodiments, rituximab is administered at 375 mg/m² dose in the first cycle and 500 mg/m² dose on Day 1 of in cycles 2-6 (every 28 days). In certain embodiments, rituximab is administered at 250 mg/m² dose. In certain embodiments, rituximab is administered at 375 mg/m² dose once weekly for 4 weeks.

In certain embodiments, rituximab first infusion is initiated a rate of 50 mg/hr; in the absence of infusion toxicity, the infusion rate is increased by 50 mg/hr increments every 30 minutes, to a maximum of 400 mg/hr. In certain embodiments, rituximab subsequent infusions are initiated a rate of 100 mg/hr; in the absence of infusion toxicity, the infusion rate is increased by 100 mg/hr increments every 30 minutes, to a maximum of 400 mg/hr. In certain embodiments, rituximab is administered as a 90 minute infusion. In certain embodiments, rituximab is administered at a rate of 20% of the total dose given in the first 30 minutes and the remaining 80% of the total dose given over the next 60 minutes. In certain embodiments, rituximab is infused at a rate of about 250 mg/hr. In certain embodiments, rituximab is infused at a rate of about 250 mg/hr for the first 30 minutes and then at about 600 mg/hr for the next 90 minutes. In certain embodiments, rituximab infusion is interrupted or the infusion rate is slowed for infusion reactions; the infusion is continued at one-half the previous rate upon improvement of symptoms.

In certain embodiments, rituximab is administered intravenously at 375 mg/m² body surface area dose per cycle, for up to 8 cycles, on day 1 of each chemotherapy cycle. In certain embodiments, rituximab is administered intravenously at 375 mg/m² body surface area dose once every 2 months until disease progression or for a maximum period of two years. In certain embodiments, rituximab is administered intravenously at 375 mg/m² body surface area dose once every 3 months until disease progression or for a maximum period of two years. In certain embodiments, rituximab is administered intravenously at 375 mg/m² body surface area dose as an intravenous infusion once weekly for four weeks. In certain embodiments, rituximab is administered intravenously at 375 mg/m² body surface area dose on day 1 of each chemotherapy cycle for 8 cycles. In certain embodiments, rituximab is administered intravenously at 375 mg/m² body surface area dose on day 0 of the first treatment cycle followed by 500 mg/m² body surface area on day 1 of each subsequent cycle for 6 cycles in total.

In certain embodiments, rituximab is administered subcutaneously. In certain embodiments, rituximab is administered subcutaneously at 1400 mg dose. In certain embodiments, rituximab is administered subcutaneously at 1600 mg dose. In certain embodiments, rituximab is administered in first cycle intravenously at 375 mg/m² body surface area dose, followed by subsequent cycles subcutaneously at a fixed dose of 1400 mg per cycle for up to 8 cycles on day 1 of each chemotherapy cycle. In certain embodiments, rituximab is administered subcutaneously at 1400 mg dose once every 3 months until disease progression or for a maximum period of two years. In certain embodiments, rituximab is administered via subcutaneous injection over approximately 5 minutes (for 1400 mg dose). In certain embodiments, rituximab is administered intravenously at 375 mg/m² body surface area dose on day 0 of the first cycle of treatment followed by subcutaneous injection at a fixed dose of 1600 mg per cycle, on day 1 of each subsequent cycle (in total: 6 cycles). In certain embodiments, rituximab is administered via subcutaneous injection over approximately 7 minutes (for 1600 mg dose).

In certain embodiments, gemcitabine is gemcitabine hydrochloride. The term gemcitabine includes gemcitabine hydrochloride. In certain embodiments, gemcitabine is administered on days 1 and 8 of a 21 day cycle at about 1000 mg/m² dose. In certain embodiments, gemcitabine is administered intravenously. In certain embodiments, gemcitabine is administered over 30 minutes. In certain embodiments, gemcitabine is administered intravenously over 30 minutes on days 1 and 8 of a 21 day cycle at about 1000 mg/m² dose.

In certain embodiments, gemcitabine is in a form of 200 mg/5.26 mL injection vial, 1 g/26.3 mL injection vial, or 2 g/52.6 mL injection vial. In certain embodiments, gemcitabine is in a form of vial(s) of gemcitabine for injection containing either 200 mg, 1 g, or 2 g of gemcitabine hydrochloride (expressed as free base). In certain embodiments, gemcitabine is in a form of 200 mg powder for solution for infusion. In certain embodiments, one vial contains gemcitabine hydrochloride equivalent to 200 mg gemcitabine. In certain embodiments, gemcitabine is in a form of 1000 mg powder for solution for infusion. In certain embodiments, one vial contains gemcitabine hydrochloride equivalent to 1000 mg gemcitabine. In certain embodiments, after reconstitution, the solution contains 38 mg/ml of gemcitabine.

In certain embodiments, gemcitabine is administered intravenously at 1000 mg/m² dose over 30 minutes on Days 1 and 8 of each 21-day cycle. In certain embodiments, gemcitabine is administered intravenously at 1250 mg/m² dose over 30 minutes on Days 1 and 8 of each 21-day cycle. In certain embodiments, gemcitabine is administered intravenously at 1000 mg/m² dose over 30 minutes on Days 1, 8, and 15 of each 28-day cycle. In certain embodiments, gemcitabine is administered intravenously at 1250 mg/m² dose over 30 minutes on Days 1 and 8 of each 21-day cycle. In certain embodiments, gemcitabine is administered intravenously at 1000 mg/m² dose over 30 minutes once weekly for up to 7 weeks (or until toxicity necessitates reducing or holding a dose), followed by a week of rest from treatment. In certain embodiments, subsequent cycles consist of infusions once weekly for 3 consecutive weeks out of every 4 weeks. In certain embodiments, dose reductions or discontinuation may be needed based on toxicities. In certain embodiments, gemcitabine dose is reduced to 50 or 75% of a full dose.

In certain embodiments, gemcitabine is administered at 1000 mg/m² dose, given by 30-minute intravenous infusion, once weekly for 3 weeks, followed by a 1-week rest period; this 4-week cycle is then repeated. In certain embodiments, dosage reduction with each cycle or within a cycle may be applied based upon the grade of toxicity experienced by the patient.

In certain embodiments, lenalidomide is administered once daily on days 1 to 21 of a 28 day cycle at about 25 mg dose. In certain embodiments, lenalidomide is administered orally. In certain embodiments, lenalidomide is administered once daily. In certain embodiments, lenalidomide is administered orally once daily on days 1 to 21 of a 28 day cycle at about 25 mg dose.

In certain embodiments, lenalidomide is in a form of 2.5 mg, 5 mg, 10 mg, 15 mg, 20 mg, or 25 mg capsules.

In certain embodiments, lenalidomide is administered at 25 mg dose once daily orally on Days 1-21 of repeated 28-day cycle. In certain embodiments, lenalidomide is administered at 10 mg dose once daily. In certain embodiments, lenalidomide is administered at 2.5 mg dose once daily. In certain embodiments, lenalidomide is administered at 5 mg dose once daily. In certain embodiments, lenalidomide is administered at 10 mg dose once daily. In certain embodiments, lenalidomide is administered at 15 mg dose once daily. In certain embodiments, lenalidomide is administered at 15 mg dose every 48 hours. In certain embodiments, lenalidomide is administered at a dose 5 mg less than the previous dose.

In certain embodiments, lenalidomide is administered at 7.5 mg dose once daily. In certain embodiments, lenalidomide is administered at 20 mg dose once daily. In certain embodiments, lenalidomide is administered at 10 mg dose once daily orally on Days 1-21 of repeated 28-day cycle, for up to 9 cycles. In certain embodiments, lenalidomide is administered at 10 mg dose once daily orally on Days 1-21 of repeated 28-day cycle until disease progression. In certain embodiments, lenalidomide is administered at 10 mg dose once daily orally on Days 1-21 of repeated 28-day cycle. In certain embodiments, lenalidomide is administered at 5 mg dose once daily on days 1-21 of repeated 28-day cycles. In certain embodiments, lenalidomide is administered at 2.5 mg dose once daily on days 1-28 of repeated 28-day cycles. In certain embodiments, lenalidomide is administered at 2.5 mg dose once every other day, days 1-28 of repeated 28-day cycles. In certain embodiments, lenalidomide is administered at 2.5 mg dose once daily on days 1-21 of repeated 28-day cycles. In certain embodiments, lenalidomide is administered at 2.5 mg dose twice a week, days 1-28 of repeated 28-day cycles.

In certain embodiments, ibrutinib is administered once daily each day of a 28 day cycle at about 560 mg dose. In certain embodiments, ibrutinib is administered orally. In certain embodiments, ibrutinib is administered once daily. In certain embodiments, ibrutinib is administered orally once daily each day of a 28 day cycle at about 560 mg dose.

In certain embodiments, ibrutinib is in a form of 140 mg capsule.

In certain embodiments, ibrutinib is administered at 560 mg dose taken orally once daily (e.g., four 140 mg capsules once daily). In certain embodiments, ibrutinib is administered at 420 mg dose taken orally once daily (e.g., three 140 mg capsules once daily). In certain embodiments, ibrutinib capsules are taken orally with a glass of water. In certain embodiments, ibrutinib is administered at 140 mg dose taken orally once daily (e.g., one 140 mg capsule once daily). In certain embodiments, ibrutinib is administered at 280 mg dose taken orally once daily (e.g., two 140 mg capsules once daily).

In certain embodiments, venetoclax is administered once daily at about 10 mg to about 400 mg dose. In certain embodiments, venetoclax is administered once daily at about 10 mg dose. In certain embodiments, venetoclax is administered once daily at about 20 mg dose. In certain embodiments, venetoclax is administered once daily at about 50 mg dose. In certain embodiments, venetoclax is administered once daily at about 100 mg dose. In certain embodiments, venetoclax is administered once daily at about 200 mg dose. In certain embodiments, venetoclax is administered once daily at about 300 mg dose. In certain embodiments, venetoclax is administered once daily at about 400 mg dose. In certain embodiments, venetoclax is administered orally.

In certain embodiments, venetoclax is in a form of 10 mg, 50 mg, or 100 mg tablets.

In certain embodiments, venetoclax is administered at 20 mg dose once daily for 7 days, followed by a weekly ramp-up dosing schedule to the recommended daily dose of 400 mg. In certain embodiments, venetoclax is administered at 20 mg dose once daily for 7 days, then at 50 mg dose once daily for 7 days, then at 100 mg dose once daily for 7 days, then at 200 mg dose once daily for 7 days, then at 400 mg dose. In certain embodiments, venetoclax is administered at a reduced dose of 10 mg (for 20 mg dose at interruption), 20 mg (for 50 mg dose at interruption), 50 mg (for 100 mg dose at interruption), 100 mg (for 200 mg dose at interruption), 200 mg (for 300 mg dose at interruption), or 300 mg (for 400 mg dose at interruption). In certain embodiments, during the ramp-up phase, the reduced dose of venetoclax is continued for 1 week before increasing the dose.

In certain embodiments, nivolumab is administered once every two weeks on day 1 and 15 of a 28-day cycle at about 3 mg/kg dose. In certain embodiments, nivolumab is administered once every two weeks at about 240 mg dose. In certain embodiments, nivolumab is administered once every two weeks on day 1 and 15 of a 28-day cycle at about 240 mg dose. In certain embodiments, nivolumab is administered intravenously.

In certain embodiments, the immunotherapy agent for use in the methods and kits provided herein is administered once every two weeks or once every three weeks. In certain embodiments, the immunotherapy agent is administered once every two weeks. In certain embodiments, the immunotherapy agent is administered once every three weeks. In certain embodiments, the immunotherapy agent is administered once every four weeks. Suitable doses of an immunotherapy agent can generally range from about 1 mg/kg to about 4 mg/kg or about 2 mg/kg to about 3 mg/kg. In certain embodiments a suitable dose of the immunotherapy agent is 2 mg/kg. In certain embodiments, a suitable dose of the immunotherapy agent is 3 mg/kg. In certain embodiments, a suitable dose of immunotherapy agent is from about 200 mg to about 300 mg. In certain embodiments, a suitable dose of immunotherapy agent is 240 mg. In certain embodiments, the immunotherapy agent is nivolumab administered at a dose of 3 mg/kg every 2 weeks, such as on days 1 and 15 of a 28 day cycle. In certain embodiments, the immunotherapy agent is nivolumab administered at a dose of 240 mg every two weeks, such as on day 1 and 15 of a 28-day cycle. In certain embodiments, the immunotherapy agent is pembrolizumab administered at a dose of 2 mg/kg every 3 weeks, such as on days 1 and 22 of a 28 day cycle. In certain embodiments, the immunotherapy agent is administered intravenously.

The therapeutically effective amount of the subject combination comprising compounds for use in the methods and kits provided herein may vary depending upon the intended application (in vitro or in vivo), or the subject and disease condition being treated, e.g., the weight and age of the subject, the severity of the disease condition, the manner of administration and the like. The term also applies to a dose that will induce a particular response in target cells, e.g., reduction of proliferation or downregulation of activity of a target protein. The specific dose will vary depending on the particular compounds chosen, the dosing regimen to be followed, whether it is administered in combination with other compounds, timing of administration, the tissue to which it is administered, and the physical delivery system in which it is carried.

The amounts or suitable doses of the methods and kits of this disclosure depends upon a number of factors, including the nature of the severity of the condition to be treated, the particular inhibitor or agent, the route of administration and the age, weight, general health, and response of the individual subject. In certain embodiments, the suitable dose level is one that achieves a therapeutic response as measured by tumor regression, or other standard measures of disease progression, progression free survival or overall survival. In certain embodiments, the suitable dose level is one that achieves this therapeutic response and also minimizes any side effects associated with the administration of the therapeutic agent. The suitable dose levels may be ones that prolong the therapeutic response and/or prolong life.

It will be understood that a suitable dose of the second therapeutic agent, the SYK inhibitor, and the additional therapeutic agent(s) may be taken at any time of the day or night. In certain embodiments, a suitable dose of each therapeutic agent is taken in the morning. In some other embodiments, a suitable dose of each therapeutic agent is taken in the evening. In certain embodiments, a suitable dose of each of the therapeutic agents is taken both in the morning and the evening. It will be understood that a suitable dose of each inhibitor may be taken with or without food. In certain embodiments a suitable dose of a therapeutic agent is taken with a meal. In certain embodiments a suitable dose of a therapeutic agent is taken while fasting.

In certain embodiments, provided herein is a method for treating DLBCL, comprising administering to a subject having DLBCL a therapeutically effective amount of a combination comprising a SYK inhibitor and a second therapeutic agent, wherein the combination further comprises one or more additional therapeutic agents.

### Pharmaceutical Compositions

In certain embodiments, the SYK inhibitor and the second agent for use in the methods and kits provided herein are both administered orally such as in a solid dosage form or a liquid dosage form. In certain embodiments, the second agent is administered as a solid dosage form. In certain embodiments, the second agent is administered as a liquid dosage form. In certain embodiments, the SYK inhibitor is administered as a solid dosage form. In certain embodiments, the SYK inhibitor is administered as a liquid dosage form.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents such as phosphates or carbonates.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules may be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. In certain embodiments, solid dosage forms may be embedding compositions that may comprise polymeric substances and waxes.

In certain embodiments, liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, cyclodextrins, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

### Medical Kits

The present disclosure also provides medical kits. In certain embodiments, the kits comprise a SYK inhibitor and a second therapeutic agent as described herein. In certain embodiments, the kits comprise a SYK inhibitor and a second therapeutic agent as described herein, in suitable packaging, and written material that can include instructions for use, discussion of clinical studies, listing of side effects, and the like. In certain embodiments, the kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. In certain embodiments, such information may be based on the results of various studies, for example, studies using experimental animals involving in vivo models and studies based on human clinical trials. In certain embodiments, the kit may further contain one or more additional therapeutic agent(s). In certain embodiments, the inhibitors of the present disclosure and the second agent are provided as separate compositions in separate containers within the kit. In certain embodiments, the inhibitors of the present disclosure and the second agent are provided as a single composition within a container in the kit. In certain embodiments, the inhibitors of the present disclosure, the second agent, and one or more additional therapeutic agent(s) are provided as separate compositions in separate containers within the kit. In certain embodiments, the inhibitors of the present disclosure, the second agent, and one or more additional therapeutic agent(s) are provided as a single composition within a container in the kit. In certain embodiments, suitable packaging and additional articles for use (e.g., measuring cup for liquid preparations, foil wrapping to minimize exposure to air, and the like) are known in the art and may be included in the kit. In certain embodiments, kits described herein can be provided, marketed and/or promoted to health providers, including physicians, nurses, pharmacists, formulary officials, and the like. In certain embodiments, kits may also be marketed directly to the consumer.

In certain embodiments, provided herein is a medical kit for treating an NHL comprising a therapeutically effective amount of a combination comprising a SYK inhibitor and a second therapeutic agent. In certain embodiments, provided herein is a medical kit for treating an NHL other than CLL comprising a therapeutically effective amount of a combination comprising a SYK inhibitor and a second therapeutic agent. In certain embodiments, provided herein is a medical kit for treating an NHL comprising a therapeutically effective amount of a combination comprising a SYK inhibitor and a second therapeutic agent other than ibrutinib, idelalisib, or fludarabine. In certain embodiments, the NHL is CLL, iNHL, MCL, PTLD, or DLBCL. In certain embodiments, the NHL is iNHL, MCL, PTLD, or DLBCL. In certain embodiments, the NHL is DLBCL. In certain embodiments, the SYK inhibitor for medical kits provided herein is a compound of Formula I, Formula II, or Formula III. In certain embodiments, the SYK inhibitor is a compound of Formula II or a pharmaceutically acceptable salt thereof. In certain embodiments, the SYK inhibitor is a compound of Formula III or a crystalline form thereof. In certain embodiments, the SYK inhibitor for medical kits provided herein is a compound of Formula III or a crystalline form thereof and the second therapeutic agent is bendamustine. In certain embodiments, the medical kits provided herein further comprise rituximab. In certain embodiments, the SYK inhibitor for medical kits provided herein is a compound of Formula III or a crystalline form thereof and the second therapeutic agent is bendamustine, wherein the medical kit further comprises rituximab. In certain embodiments, the SYK inhibitor for medical kits provided herein is a compound of Formula III or a crystalline form thereof and the second therapeutic agent is gemcitabine. In certain embodiments, the SYK inhibitor for medical kits provided herein is a compound of Formula III or a crystalline form thereof and the second therapeutic agent is lenalidomide. In certain embodiments, the SYK inhibitor for medical kits provided herein is a compound of Formula III or a crystalline form thereof and the second therapeutic agent is ibrutinib. In certain embodiments, the SYK inhibitor for medical kits provided herein is a compound of Formula III or a crystalline form thereof and the second therapeutic agent is venetoclax. In certain embodiments, the SYK inhibitor for medical kits provided herein is a compound of Formula III or a crystalline form thereof and the second therapeutic agent is nivolumab.

### Further Combination Therapies

The present invention also provides methods for further combination therapies in which, in addition to a SYK inhibitor and a second therapeutic agent, one or more agents known to modulate other pathways, or the same pathway, may be used. In certain embodiments, such therapy includes but is not limited to the combination of the composition comprising at least one SYK inhibitor and at least one second therapeutic agent, as described herein, with one or more additional therapeutic agents such as anticancer agents, chemotherapeutic agents, therapeutic antibodies, and radiation treatment, to provide, where desired, a synergistic or additive therapeutic effect. Pathways that may be targeted by administering an additional agent include, but are not limited to, spleen tyrosine kinase (SYK), MAP kinase, Raf kinases, Akt, NFkB, WNT, RAS/RAF/MEK/ERK, JNK/SAPK, p38 MAPK, Src Family Kinases, JAK/STAT and/or PKC signaling pathways. Additional agents may target one or more members of one or more signaling pathways. Representative members of the nuclear factor-kappaB (NFkB) pathway include but are not limited to RelA (p65), RelB, c-Rel, p50/p105 (NF-cB 1), p52/p 100 (NF-κB2), IkB, and IkB kinase. Non-limiting examples of receptor tyrosine kinases that are members of the phosphatidylinositol 3-kinase (PI3K)/AKT pathway that may be targeted by one or more agents include FLT3 LIGAND, EGFR, IGF-1R, HER2/neu, VEGFR, and PDGFR. Downstream members of the PI3K/AKT pathway that may be targeted by agents according to the methods of the invention include, but are not limited to, forkhead box O transcription factors, Bad, GSK-313, I-κB, mTOR, MDM-2, and S6 ribosomal subunit.

### EXAMPLES

### Example 1

Antitumor activity of Compound A and anti-PD-1 (administered orally or intraperitoneally, respectively) alone or Compound A combined with anti-PD-1 in female Balb/c mice bearing A20 mouse syngeneic B-cell lymphoma.

A20 tumor cells were inoculated subcutaneously to Balb/C mice. The treatment started when the tumors reached a mean volume of 80 mm³. Compound A at 60 mg/kg was administered daily (QD) PO for 21 consecutive days. Anti-PD-1 at 10 mg/kg was administered every 4 days (Q4D) for 3 doses in total. Compound A at 60 mg/kg combined with anti-PD-1 at 10 mg/kg was administered following the same dosing regimen as the single agents.

The results of the pairwise comparison to vehicle showed that the daily administration of Compound A alone at 60 mg/kg or anti-PD-1 alone at 10 mg/kg resulted in tumor growth inhibition (TGI) values on Day 19 of 15.3% (ΔAUC, p>0.05) and 7.4% (ΔAUC, p>0.05), respectively. Compound A at 60 mg/kg combined with anti-PD-1 at 10 mg/kg showed additive anti-tumor activity and had 51.7% (ΔAUC, p<0.05) TGI value on Day 19.

During the treatment period from Day 0 to Day 21, no mortality or major body weight loss (BWL) was observed in the study. Several mice were removed from the study during the treatment period because the tumor volumes reached the end point (>2000 mm³). Compound A combined with anti-PD-1 had additive effect in the A20 B-cell syngeneic mouse model. Treatments with the single agents or combination were well tolerated by the mice.

Experimental design: Female Balb/c mice (Charles River; weight at treatment start was 19.5 g) were inoculated subcutaneously in the flank (cell suspension) with 5.0×10⁶ A20 cells with Matrigel™. Tumor growth was monitored with vernier calipers. Tumor volume was calculated using the formula V=W²×L/2, where V=volume, W=width and L=length of the tumor. When the mean tumor volume reached approximately 80 mm³, the animals were randomized into treatment groups (n=8/group). Mice were then dosed with 0.5% methylcellulose or Compound A or anti-PD-1 over a 21 day period (see Table 1 for details). Tumor growth and body weight were measured twice per week. Tumor growth inhibition and body weight change were calculated on Day 19 of treatment.

Statistical analysis: The differences in the tumor growth trends over time between pairs of treatment groups were assessed using linear mixed effects regression models. These models account for the fact that each animal was measured at multiple time points. A separate model was fit for each comparison, and the areas under the curve (AUC) for each treatment group were calculated using the predicted values from the model. The percent decrease in AUC (dAUC) relative to the reference group was then calculated. A statistically significant P value suggests that the trends over time for the two treatment groups were different. The results of pairwise comparison are summarized in Table 2. Further details regarding pairwise comparisons are provided in the Example 2.

Drug combinations were assessed for synergy using observed AUC values. The change in AUC relative to the control was calculated for both single agent treatment groups as well as the combination group. The interaction between the two compounds was then assessed by comparing the change in AUC observed in the combination group to the sum of the changes observed in both single agents. The results can be divided into four categories: synergistic, additive, sub-additive, and antagonistic. The results of synergy assessment are summarized in Table 3. Further details regarding combination analysis are provided in the Example 2.

Once a final analysis was selected, the tumor measurements observed on a date pre-specified by the researcher (typically the last day of treatment) were analyzed to assess tumor growth inhibition. For this analysis, a treatment over control (T/C) ratio was calculated for each animal by dividing the tumor measurement for the given animal by the mean tumor measurement across all control animals. The T/C ratios across a treatment group were compared to the T/C ratios of the control group using a two-tailed Welch's t-test.

All P values <0.05 were called statistically significant in this Example. Days greater than 19 were excluded. The number of animals removed from this study is shown in Table 1.

Results and discussion: The results of the pairwise comparison to vehicle showed that the daily administration of Compound A alone at 60 mg/kg or anti-PD-1 alone at 10 mg/kg resulted in tumor growth inhibition (TGI) values on Day 19 of 15.3% (ΔAUC, p>0.05) and 7.4% (ΔAUC, p>0.05), respectively. Compound A at 60 mg/kg combined with anti-PD-1 at 10 mg/kg showed additive anti-tumor activity and had 51.7% (ΔAUC, p<0.05) TGI value on Day 19. All the TGI calculation was completed on day 19 due to the faster tumor growth rate.

In the present study, on Day 21, the mean body weight of the vehicle group increased 9.7% compared to Day 0 when the animals were grouped. There were 4 mice terminated and removed from vehicle group at day 14 due the bigger tumor volumes (>2000 mm³). For animals treated with Compound A alone at 60 mg/kg (PO, QD×21 days) and anti-PD-1 alone at 10 mg/kg (IP, Q4D×3), the mean body weight increased by 15.8% and 17.3%, respectively, on Day 19 as compared to Day 0.

For combination treatment group, no BWL was observed in the animals treated with Compound A at 60 mg/kg (PO, QD×21 days) combined with anti-PD-1(IP, Q4D×3). The mean body weight of this group increased by 11.2% on Day 19 as compared to Day 0. No mouse was removed from the study during the treatment period. All of the mice in the combination group tolerated the treatment well.

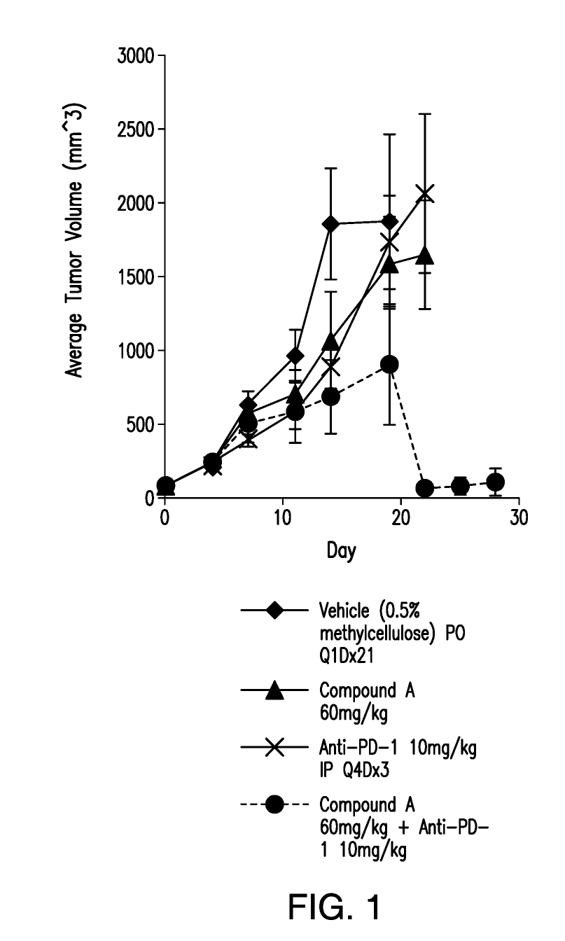

The antitumor activities of Compound A, anti-PD-1, and combination of Compound A with anti-PD-1 against A20 mouse syngeneic B-cell lymphoma model are summarized in Table 1 and graphically presented in FIG. 1. The results of pairwise comparison are summarized in Table 2. Results of the combination analysis are summarized in Table 3.

TABLE 1
Tumor growth inhibition.

| | | | | | | Mean | | | |
|---|---|---|---|---|---|---|---|---|---|
| | | | | % BW^{a} | Number | Tumor | | | |
| | | | Route | Change | of | Volume | | | |
| | Dose | | Schedule | (Day | animals | (mm³) ± | | TGI^{d} | P-Value^{e} |
| Test Article | (mg/kg) | Sequencing | Duration | Maximum) | removed | SEM^{b} | T/C^{c} | (%) | (dAUC) |
| 0.5% methyl- | — | — | PO QD 21 | −0.6 (4) | 4 | 1,869.8 ± 589.2 | N/A | N/A | N/A |
| cellulose | | | | | | | | | |
| Compound A | 60.0 | — | PO QD 21 | 15.8 (19) | 1 | 1,582.9 ± 317.2 | 0.85 | 15.3 | >0.05 |
| anti-PD-1 | 10.0 | — | IP Q4D 21 | 17.3 (19) | 0 | 1,730.7 ± 316.8 | 0.93 | 7.4 | >0.05 |
| Compound A; | 60.0; | Compound A// | PO QD 21; | 11.2 (19) | 0 | 903.3 ± 408.1 | 0.48 | 51.7 | <0.05 |
| anti-PD-1 | 10.0 | anti-PD-1 | IP Q4D 21 | | | | | | |

TGI and T/C values were calculated on Day 19 of treatment.
^{a}% Body weight change (Day of Maximum change within the treatment period).
^{b}Standard error of the mean.
^{c}Treatment over control.
^{d}TGI = 100 − [(treated average volume/control average volume) × 100].
^{e}The changes in the areas under the tumor volume versus time curves (ΔAUCs) were assessed using linear mixed effects regression models to compare treatment groups with vehicle. P-values <0.05 indicate the percent decrease in AUC (dAUC) relative to the reference group was statistically significant.

TABLE 2
Results of pairwise comparison.

| Reference | Treated | dAUC | P-Value |
|---|---|---|---|
| 0.5% methylcellulose | Compound A | 10.2 | >0.05 |
| 0.5% methylcellulose | anti-PD-1 | 13.1 | >0.05 |
| 0.5% methylcellulose | Compound A; | 78.4 | <0.05 |
| | anti-PD-1 | | |

TABLE 3
Results of combination analysis.

| Test Article | Score | SEM | P-Value | Assess |
|---|---|---|---|---|
| Compound A; anti-PD-1 | −39.5 | 24.0 | 0.133 | Additive |

Compound A combined with anti-PD-1 has additive effect in the A20 B-cell syngeneic mouse model.

### Example 2

Statistical methodology for Example 1-Example 14.

All tumor values (tumor volumes or photon flux) had a value of 1 added to them before log₁₀ transformation. These values were compared across treatment groups to assess whether the differences in the trends over time were statistically significant. To compare pairs of treatment groups, the following mixed-effects linear regression model was fit to the data using the maximum likelihood method:

Yᵢⱼₖ−Yᵢ₀ₖ=Yᵢ₀ₖ+treatᵢ+dayⱼ+dayⱼ²+(treat*day)ᵢⱼ+(treat*day²)ᵢⱼ+εᵢⱼₖ

where Yᵢⱼₖ is the log₁₀ tumor value at the j^{th} time point of the k^{th} animal in the i^{th} treatment, Yᵢ₀ₖ is the day 0 (baseline) log₁₀ tumor value in the k^{th} animal in the i^{th} treatment, dayⱼ was the median-centered time point and (along with dayⱼ²) was treated as a continuous variable, and εᵢⱼₖ is the residual error. A spatial power law covariance matrix was used to account for the repeated measurements on the same animal over time. Interaction terms as well as dayⱼ² terms were removed if they were not statistically significant.

A likelihood ratio test was used to assess whether a given pair of treatment groups exhibited differences which were statistically significant. The −2 log likelihood of the full model was compared to one without any treatment terms (reduced model) and the difference in the values was tested using a Chi-squared test. The degrees of freedom of the test were calculated as the difference between the degrees of freedom of the full model and that of the reduced model.

The predicted differences in the log tumor values (Yᵢⱼₖ−Yᵢ₀ₖ, which can be interpreted as log₁₀ (fold change from day 0)) were taken from the above models to calculate mean AUC values for each treatment group. A dAUC value was then calculated as:

$$dAUC = \frac{mean ( {AUC}_{ctl} ) - mean ( {AUC}_{trt} )}{mean ( {AUC}_{ctl} )} * 100$$

This assumes AUC_{ctl} was positive. In instances where AUC_{Ctl} was negative, the above formula was multiplied by −1.

For synergy analyses, the observed differences in the log tumor values were used to calculate AUC values for each animal. In instances when an animal in a treatment group was removed from the study, the last observed tumor value was carried forward through all subsequent time points. The AUC for the control, or vehicle, group was calculated using the predicted values from the pairwise models described above. To address the question of whether the effects of the combination treatments were synergistic, additive, sub-additive, or antagonistic relative to the individual treatments, the following statistics were calculated:

$${Frac}_{{A}_{k}} = \frac{{AUC}_{ctl} - {AUC}_{{A}_{k}}}{{AUC}_{ctl}}$$

$${Frac}_{{B}_{k}} = \frac{{AUC}_{ctl} - {AUC}_{{B}_{k}}}{{AUC}_{ctl}}$$

$${Frac}_{{AB}_{k}} = \frac{{AUC}_{ctl} - {AUC}_{{AB}_{k}}}{{AUC}_{ctl}}$$

$$Synergy Score = ( mean ( {Frac}_{A} ) + mean ( {Frac}_{B} ) - mean ( {Frac}_{AB} ) ) * 100$$

where Aₖ and Bₖ are the k^{th} animal in the individual treatment groups and ABₖ is the k^{th} animal in combination treatment group. AUC_{ctl} is the model-predicted AUC for the control group and was treated as a constant with no variability. The standard error of the synergy score was calculated as the square root of the sum of squared standard errors across groups A, B, and AB. The degrees of freedom were estimated using the Welch-Satterthwaite equation. A hypothesis test was performed to determine if the synergy score differed from 0. P values were calculated by dividing the synergy score by its standard error and tested against a t-distribution (two-tailed) with the above-calculated degrees of freedom. The effect of the combination treatment was considered synergistic if the synergy score was less than 0 and additive if the synergy score wasn't statistically different from 0. If the synergy score was greater than zero, but the mean AUC for the combination was lower than the lowest mean AUC among the two single agent treatments, then the combination was sub-additive. If the synergy score was greater than zero, and the mean AUC for the combination was greater than the mean AUC for at least one of the single agent treatments, then the combination was antagonistic.

Given the exploratory nature of this study, there were no adjustments pre-specified for the multiple comparisons and endpoints examined in the pairwise comparisons or combination analyses. All P values <0.05 in these analyses were called statistically significant.

### Example 3

Antitumor activity of Compound A and bendamustine administered (orally, intravenously) alone or Compound A combined with bendamustine to female CB17 SCID mice bearing TMD8 DLBCL xenografts.

Compound A, bendamustine, or vehicle were administered to female SCID mice bearing TMD8 DLBCL xenografts beginning on Day 1 for 14 days. Tumor growth inhibition was calculated on Day 14 of the study. The last measurement was taken on Day 22 of the study.

Compound A was administered once daily (QD), oral (PO), at 60 mg/kg, which resulted in TGI=38.5% (p<0.01). Bendamustine was administered intravenously (IV) twice weekly on Tuesdays and Fridays (BIW) at 1 mg/kg which resulted in TGI=13.4% (p<0.05). Compound A 60 mg/kg was administered in combination with bendamustine 1 mg/kg and the combination activity was found to be additive with the overall antitumor activity being greater than that of either single agent (TGI=55.2%, p<0.001).

All of the groups were well tolerated with no animals lost, and no body weight loss throughout the study. The combination of Compound A with bendamustine did yield an additive response and improved antitumor activity over that of the single agents.

Experimental design: Female CB17 SCID mice (Taconic Biosciences; weight at treatment start was about 19 g) were inoculated subcutaneously in the flank (cell suspension) with 5.0×10⁶ TMD8 cells. Tumor growth was monitored with vernier calipers. Tumor volume was calculated using the formula V=W²×L/2, where V=volume, W=width and L=length of the tumor. When the mean tumor volume reached approximately 210 mm³, the animals were randomized into treatment groups (n=8/group). Mice were then dosed with 0.5% methylcellulose, Compound A, or bendamustine over a 14 day period (see Table 4 for details). Tumor growth and body weight were measured twice per week. Tumor growth inhibition and body weight change were calculated on Day 14 of treatment.

Statistical analysis: The differences in the tumor growth trends over time between pairs of treatment groups were assessed using linear mixed effects regression models. These models account for the fact that each animal was measured at multiple time points. A separate model was fit for each comparison, and the areas under the curve (AUC) for each treatment group were calculated using the predicted values from the model. The percent decrease in AUC (dAUC) relative to the reference group was then calculated. A statistically significant P value suggests that the trends over time for the two treatment groups were different. Further details regarding pairwise comparisons are provided in the Example 2.

Drug combinations were assessed for synergy using observed AUC values. The change in AUC relative to the control was calculated for both single agent treatment groups as well as the combination group. The interaction between the two compounds was then assessed by comparing the change in AUC observed in the combination group to the sum of the changes observed in both single agents. The results can be divided into four categories: synergistic, additive, sub-additive, and antagonistic. The results of synergy assessment are summarized in Table 5. Further details regarding combination analysis are provided in the Example 2.

Once a final analysis was selected, the tumor measurements observed on a date pre-specified by the researcher (typically the last day of treatment) were analyzed to assess tumor growth inhibition. For this analysis, a T/C ratio was calculated for each animal by dividing the tumor measurement for the given animal by the mean tumor measurement across all control animals. The T/C ratios across a treatment group were compared to the T/C ratios of the control group using a two-tailed Welch's t-test.

All P values <0.05 were called statistically significant in this Example. Days greater than 14 were excluded. All animals were included.

Results and discussion: Compound A, bendamustine, or vehicle were administered to female SCID mice bearing TMD8 DLBCL xenografts beginning on Day 1 for 14 days. Tumor growth inhibition was calculated on Day 14 of the study. The last measurement was taken on Day 22 of the study.

Compound A was administered once daily (QD), oral (PO), at 60 mg/kg, which resulted in TGI=38.5% (p<0.01). Bendamustine was administered intravenously (IV) twice weekly on Tuesdays and Fridays (BIW) at 1 mg/kg, which resulted in TGI=13.4% (p<0.05). Compound A 60 mg/kg was administered in combination with bendamustine 1 mg/kg and the combination activity was found to be additive with the overall antitumor activity being greater than that of either single agent (TGI=55.2%, p<0.001).

All of the groups were well tolerated with no animals lost, and no body weight loss throughout the study. No animals were removed from the study.

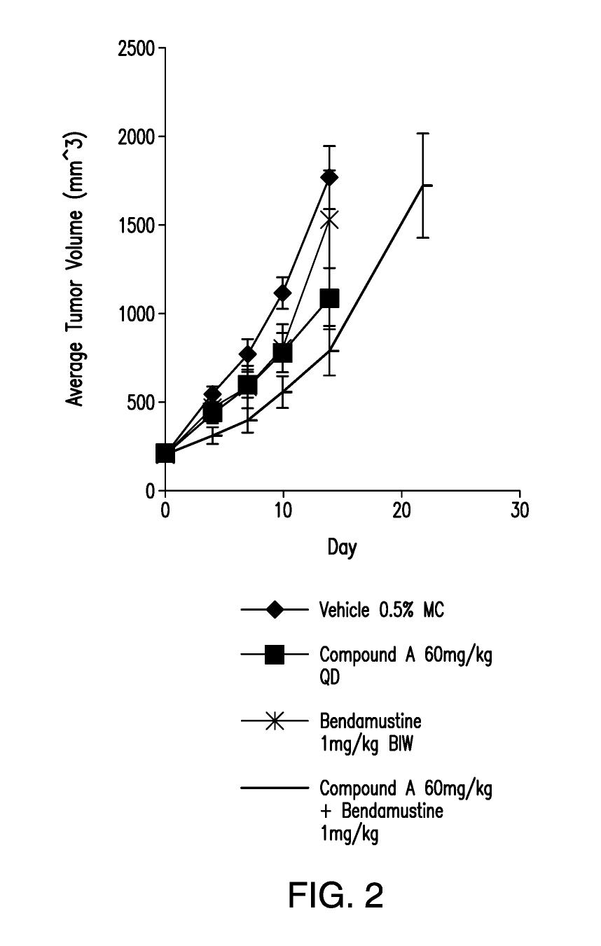

The antitumor activities of Compound A, bendamustine, and combination of Compound A with bendamustine on CB17 SCID mice bearing TMD8 DLBCL xenografts are summarized in Table 4 and graphically presented in FIG. 2.

TABLE 4
Tumor growth inhibition.

| | | | | | | Mean | | | |
|---|---|---|---|---|---|---|---|---|---|
| | | | | % BW^{a} | Number | Tumor | | | |
| | | | Route | Change | of | Volume | | | P- |
| | Dose | | Schedule | (Day | animals | (mm³) ± | | TGI^{d} | Value^{e} |
| Test Article | (mg/kg) | Sequencing | Duration | Maximum) | removed | SEM^{b} | T/C^{c} | (%) | (dAUC) |
| 0.5% | N/A | | PO QD | 10.3 (14) | 0 | 1,766.1 ± 178.5 | N/A | N/A | N/A |
| methyl- | | | 14 | | | | | | |
| cellulose | | | | | | | | | |
| Compound A | 60.0 | | PO QD | 10.3 (14) | 0 | 1,085.5 ± 173.3 | 0.61 | 38.5 | 0.002 |
| | | | 14 | | | | | | |
| bendamustine | 1.0 | | IV BIW | 13.1 (14) | 0 | 1,529.2 ± 275.9 | 0.87 | 13.4 | 0.042 |
| | | | 14 | | | | | | |
| Compound | 60.0; | Compound | PO QD | 10.7 (14) | 0 | 791 ± 138.8 | 0.45 | 55.2 | <0.001 |
| A; | 1.0 | A// | 14; IV | | | | | | |
| bendamustine | | bendamustine | BIW 14 | | | | | | |

TGI and T/C values were calculated on Day 14 of treatment.
^{a}% Body weight change (Day of Maximum change within the treatment period).
^{b}Standard error of the mean.
^{c}Treatment over control.
^{d}TGI = 100 − [(treated average volume/control average volume) × 100].
^{e}The changes in the areas under the tumor volume versus time curves (ΔAUCs) were assessed using linear mixed effects regression models to compare treatment groups with vehicle. P-values <0.05 indicate the percent decrease in AUC (dAUC) relative to the reference group was statistically significant.

TABLE 5
Combination comparisons (log-transformed).

| Comparison | Score | SEM | P-Value | Assess |
|---|---|---|---|---|
| Compound A + bendamustine | −3.2 | 17.9 | 0.859 | Additive |

### Example 4

Antitumor activity of Compound A and bendamustine alone or Compound A combined with bendamustine in female SCID mice bearing Ly19 xenografts.

In the present study, the antitumor activity of Compound A and bendamustine alone or Compound A in combination with bendamustine was evaluated in female SCID mice bearing subcutaneously (SC) implanted Ly19 xenografts. Compound A (at 60 mg/kg) was administered by oral gavage (PO) daily for 14 days (QD×14). Bendamustine (at 1.0 or 2.0 mg/kg) was given intravenously (IV) twice a week for two weeks (BIW×2). The results of the pairwise comparisons showed that the treatment with Compound A at 60 mg/kg alone or in combination with 2.0 mg/kg of bendamustine resulted in tumor growth inhibition (TGI) values of 32.6% and 52.1%, respectively. On the other hand, bendamustine alone or Compound A at 60 mg/kg combined with bendamustine at 1.0 mg/kg had TGI values between 14.6% and 22.3%. The interactions between Compound A and bendamustine were either antagonistic or additive. Female SCID mice bearing Ly19 xenografts could tolerate the treatment with Compound A or bendamustine with only transient body weight losses and without death whether the compounds were given alone or in combination.

Test and control articles: Compound A: purity >99% by weight; solid, white to off-white powder; storage conditions=room temperature. Bendamustine HCl for injection: powder (100 mg/vial); storage conditions=room temperature. The vehicle for Compound A was 0.5% methylcellulose. The vehicle for bendamustine was 0.9% saline.

The dosing solutions preparations are summarized in Table 6.

TABLE 6
Compound A dosing solution preparation (for one week) and bendamustine dosing
solution preparation (for one day).

| | | Dose | | | | |
|---|---|---|---|---|---|---|
| Animals | Compound | (mg/kg) | Volume (mL/kg) | Conc. (mg/mL) | Route | Dosing Schedule |
| 24 | Compound A | 60 | 10 | 6.0 | PO | QD × 14 |
| 16 | bendamustine | 2 | 10 | 0.2 | IV | BIW × 3 |
| 16 | bendamustine | 1 | 10 | 0.1 | IV | BIW × 3 |

The required volume for Compound A (for one week) was calculated as follows: 24 animals×20 g×10 mL/kg/1000×7×1.5=50.4 mL. Vehicle: 0.5% methylcellulose. The procedure for making Compound A at 6.0 mg/mL 50 mL solution was: (1) weigh 468 mg of Compound A powder; (2) add the powder in 50.0 mL of 0.5% methylcellulose; (3) sonicate the resulting off-white suspension for 5 minutes at room temperature and then vortex for 30 minutes; (4) check the pH and the value shall be pH=3.5; (5) store at room temperature and use it to dose 24 animals for a week. Vortex the solutions well before each dosing.

The required volume for one day of dosing of bendamustine is calculated as follows: 16 animals×20 g×10 mL/kg/1000×1.5=4.8 mL. Each bottle of bendamustine contains 100 mg of active compound. The procedure for making bendamustine solution was: (1) collect all the powders from one bottle of bendamustine and evenly distribute into 10 vials (e.g., each vial contains 10 mg of bendamustine); (2) cover the vial with aluminum foil to avoid light and store at room temperature; (3) for bendamustine (1 mg/mL) 10 mL—(a) take one vial of bendamustine (10 mg) prepared above and add 10 mL of sterile water; (b) dissolve all the powder to generate a solution of 1 mg/mL; (4) for bendamustine (0.2 mg/mL) 5.0 mL—(a) take 1.0 mL of bendamustine solution prepared in (3), (b) add 4.0 mL of sterile water, (c) use it to dose 16 animals within 3 hours; (5) for bendamustine (0.1 mg/mL) 5.0 mL—(a) take 0.5 mL of bendamustine solution prepared in (3), (b) add 4.5 mL of sterile water, (c) use it to dose 16 animals within 3 hours.

Dosing regimen: Table 7 shows the dosing regimens for each treatment group used in the study. Vehicle (0.5% methylcellulose) or Compound A were administered PO daily (QD×14). Bendamustine was administered IV (BIW×2) on Day 1, 4, 8 and 11. Dosing was initiated on Day 1 and continued up to Day 14 for animals completing the planned treatment regimen.

TABLE 7
Dosing regimen.

| | | | | | | Route and |
|---|---|---|---|---|---|---|
| Group | N | Test Article | Dose (mg/kg) | Volume (mL/kg) | Conc. (mg/mL) | Schedule |
| 1 | 8 | 0.5% methylcellulose | N/A | 10 | N/A | PO, QD × 14 |
| 2 | 8 | Compound A | 60 | 10 | 6.0 | PO, QD × 14 |
| 3 | 8 | bendamustine | 1.0 | 10 | 0.1 | IV, BIW × 2 |
| 4 | 8 | bendamustine | 2.0 | 10 | 0.2 | IV, BIW × 2 |
| 5 | 8 | Compound A | 60 | 10 | 6.0 | PO, QD × 14 |
| | | bendamustine | 1.0 | 10 | 0.1 | IV, BIW × 2 |
| 6 | 8 | Compound A | 60 | 10 | 6.0 | PO, QD × 14 |
| | | bendamustine | 2.0 | 10 | 0.2 | IV, BIW × 2 |

Data collection: Each animal (female SCID mice from Beijing HFK Bioscience Co., Ltd.; group average weight at Day 0 was 19.3-20.6 g) was inoculated with 2×10⁶ Ly19 tumor cells (in 0.1 mL, 1:1 with Matrigel™) at the right flank for tumor model development. Body weight and tumor growth were monitored twice a week. Tumor size was measured to the nearest 0.1 mm using vernier calipers and applying the formula: V=W²×L/2, where V=volume, W=width and L=length of the tumor xenograft. Xenografts were allowed to grow until they reached an average size of approximately 130 mm³ after 5 days. Mice bearing the proper size xenograft were randomly assigned into one of the eight groups shown in Table 7 and began treatment with their assigned test material, either 0.5% methylcellulose, Compound A (60 mg/kg), bendamustine (1.0 or 2.0 mg/kg), ibrutinib (6 or 20 mg/kg), or Compound A plus bendamustine for up to 14 days.

For this study, passage was 17. The study was terminated on Day 17.

Statistical tests: The differences in the tumor growth trends over time between pairs of treatment groups were assessed using linear mixed effects regression models. These models account for the fact that each animal was measured at multiple time points. A separate model was fit for each comparison, and the area under the curve (AUC) for each treatment group was calculated using the predicted values from the model. The percent decrease in AUC (dAUC) relative to the reference group was then calculated. A statistically significant P value suggests that the change over time for the two treatment groups was different. Further details regarding pairwise comparisons are provided in the Example 2.

Drug combinations were assessed for synergy using observed AUC values. The change in AUC relative to the control was calculated for both single agent treatment groups as well as the combination group. The interaction between the two compounds was then assessed by comparing the change in AUC observed in the combination group to the sum of the changes observed in both single agents. Statistically significant negative synergy scores indicate a synergistic combination (“Syn.”). Statistically significant positive synergy scores indicate a sub-additive or antagonistic combination (“Antag.”). Scores that are not statistically significant should be considered additive (“Add.”). All P values <0.05 were called statistically significant in this Example. Further details regarding pairwise comparisons are provided in the Example 2.

Results and discussion: Due to the fast growth of Ly19 xenografts, the treatment lasted only 14 days and the study was terminated on Day 17.

In the present study, no body weight loss was observed in female SCID mice bearing Ly19 xenografts from the vehicle-treated (0.5% methylcellulose, PO, QD×14) control group. On Day 14, the mean body weight of the vehicle group increased 15.8% (or 3.2 g) compared to Day 0. The maximal decrease in mean body weight was 1.2% (or 0.3 g, Day 3) in animals treated with Compound A alone at 60 mg/kg (PO, QD×14) and on Day 14, the mean body weight of this group increased 8.6% (or 1.6 g) compared to Day 0. Compound A alone at 60 mg/kg had TGI value of 32.6% (dAUC=18.8, P<0.01). No body weight loss was observed in animals treated with bendamustine alone at 1.0 or 2.0 mg/kg (IV, BIW×2). Nevertheless, bendamustine had TGI values of 16.2% (dAUC=10.9, P<0.05) and 18.7% (dAUC=11.9, P>0.05), respectively.

The maximal decrease in mean body weight was 0.8% (or 0.2 g, Day 3) and 2.9% (or 0.5 g, Day 3) in animals treated with 60 mg/kg of Compound A in combination with bendamustine at 1.0 or 2.0 mg/kg, respectively. The combination of Compound A at 60 mg/kg and bendamustine at 1.0 mg/kg had TGI value of 14.6% (dAUC=9.0, P>0.05) and antagonistic effect (Score=20.8, P<0.05). On the other hand, additive effect (Score=1.2, P>0.05) was observed when Compound A at 60 mg/kg was administered in combination with bendamustine at 2.0 mg/kg; TGI value was 52.1% (dAUC=31.6, P<0.01).

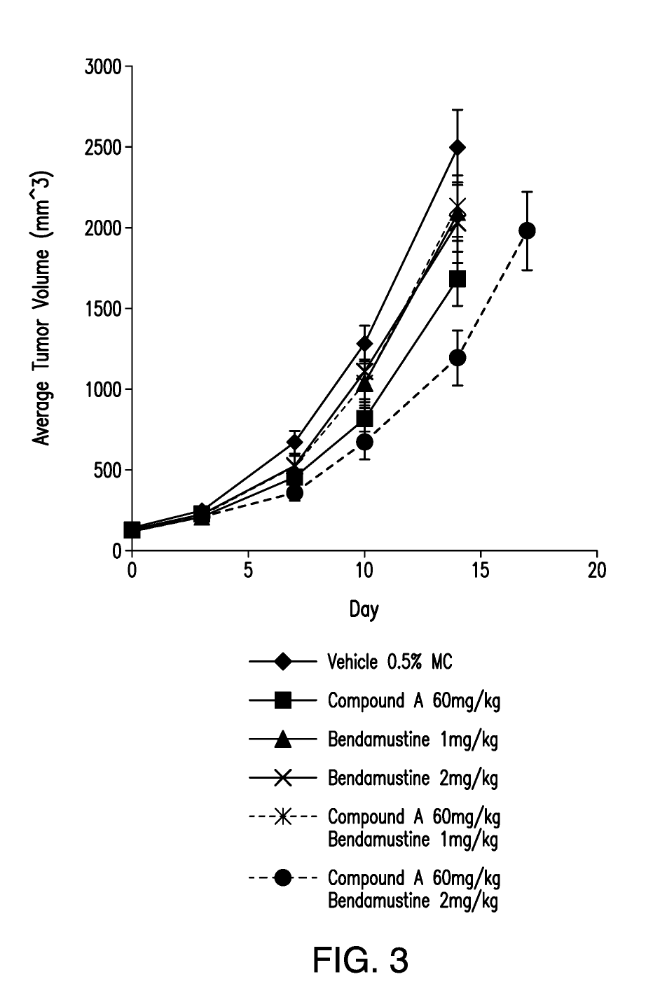

Changes in animal body weight following the administration of Compound A and bendamustine alone or Compound A in combination with bendamustine are summarized in Table 8. The antitumor activity of Compound A and bendamustine alone or Compound A in combination with bendamustine against Ly19 xenografts is summarized in Table 9 and graphically presented in FIG. 3. Results of the combination analysis are summarized in Table 10.

TABLE 8
Effects of Compound A and bendamustine on animal body weight (g).

| Group | Day 0 | Day 3 | Day 7 | Day 10 | Day 14 |
|---|---|---|---|---|---|
| 0.5% methylcellulose (QD × 14) | 20.3 | 20.7 | 21.5 | 22.3 | 23.5 |
| | Change (%) | +1.9 | +5.9 | +9.8 | +15.8 |
| Compound A (60 mg/kg, QD × 14) | 19.6 | 19.3 | 19.9 | 20.4 | 21.2 |
| | Change (%) | −1.2 | +1.7 | +4.2 | +8.6 |
| bendamustine (1.0 mg/kg, BIW × 2) | 19.7 | 19.9 | 20.4 | 21.2 | 22.3 |
| | Change (%) | +0.9 | +3.7 | +7.7 | +13.1 |
| bendamustine (2.0 mg/kg, BIW × 2) | 20.4 | 20.8 | 21.1 | 21.7 | 22.6 |
| | Change (%) | +2.0 | +3.2 | +6.2 | +10.8 |
| Compound A (60 mg/kg) + | 20.6 | 20.4 | 20.8 | 21.1 | 22.0 |
| bendamustine (1.0 mg/kg) | Change (%) | −0.8 | +1.0 | +2.6 | +7.3 |
| Compound A (60 mg/kg) + | 20.1 | 19.6 | 20.1 | 20.5 | 21.3 |
| bendamustine (2.0 mg/kg) | Change (%) | −2.9 | +0.0 | +2.1 | +5.8 |

Data is presented as Mean of 8 animals in each group.

TABLE 9
Effects of Compound A and bendamustine on tumor growth.

| | Mean Tumor Volume (mm³) ± | | | P Value |
|---|---|---|---|---|
| Group | SEM at Day 14^{a} | TGI^{b} (%) | dAUC | (dAUC)^{c} |
| 0.5% methylcellulose (QD × 14) | 2496.9 ± 232.7 | N/A | N/A | N/A |
| Compound A (60 mg/kg, QD × 14) | 1683.9 ± 167.1 | 32.6 | 18.8 | <0.01 |
| bendamustine (1.0 mg/kg, BIW × 2) | 2092.9 ± 175.8 | 16.2 | 10.9 | <0.05 |
| bendamustine (2.0 mg/kg, BIW × 2) | 2029.8 ± 248.8 | 18.7 | 11.9 | >0.05 |
| Compound A (60 mg/kg) + | 2132.7 ± 191.0 | 14.6 | 9.0 | >0.05 |
| bendamustine (1.0 mg/kg) | | | | |
| Compound A (60 mg/kg) + | 1195.3 ± 169.6 | 52.1 | 31.6 | <0.01 |
| bendamustine (2.0 mg/kg) | | | | |

^{a}Data presented as Mean ± SEM of 8 animals in each group.
^{b}TGI = (V_{vehicle} − Vₜᵣₑₐₜₘₑₙₜ)/V_{vehicle} × 100% and values were calculated based on the measurements on Day 14.
^{c}The changes in the areas under the tumor volume versus time curves (ΔAUCs) were assessed using linear mixed effects regression models to compare treatment groups with vehicle. P-values <0.05 indicate the percent decrease in AUC (dAUC) relative to the reference group was statistically significant.

TABLE 10
Results of combination analysis.

| Comparison | Score | SEM | P-Value | Assess |
|---|---|---|---|---|
| Compound A (60 mg/kg) + | 20.8 | 9.3 | <0.05 | Antag. |
| bendamustine (1.0 mg/kg) | | | | |
| Compound A (60 mg/kg) + | 1.2 | 11.4 | >0.05 | Add. |
| bendamustine (2.0 mg/kg) | | | | |

The antitumor activity of Compound A combined with bendamustine showed antagonistic or additive effect. Animals tolerate the treatment with Compound A and bendamustine well whether given alone or in combination.

### Example 5

Antitumor activity of Compound A, ibrutinib, or bendamustine alone or in combination in female SCID mice bearing OCI-LY10 human DLBCL xenografts.

Mice were inoculated subcutaneously (SC) into the right flank with OCI-Ly10 human DLBCL cells and were treated once daily (QD) with oral (PO) doses of vehicle, or Compound A. Ibrutinib was administered QD PO and bendamustine was administered twice weekly (BIW) intravenously (IV) as single agents or in combination with Compound A for 21 days. Effects on tumor growth were evaluated by measuring percent tumor growth inhibition (TGI). Tolerability was assessed by percent body weight loss (BWL), lethality and clinical signs of adverse treatment-related side effects. Body weights were measured BIW. The percentage TGI was determined on Day 21. Statistical comparisons of tumor growth between treatment groups and vehicle were conducted to assess antitumor activity using a linear mixed effects regression analysis on the change in the area under the tumor volume-time curve (ΔAUC). P-value less than 0.05 was considered statistically significant. Synergy analysis was conducted to evaluate the effects of combination treatment compared to single-agent treatment alone. See Example 2 for further details.

Compound A was administered QD, PO, at 60 mg/kg which resulted in TGI of 38.8% (ΔAUC, p<0.001). Ibrutinib was administered QD, PO, at 6 mg/kg, and was found to have TGI=18.0% (ΔAUC, p<0.01), when compared to vehicle. Bendamustine, administered IV, on a BIW schedule (6 doses) at 1 mg/kg, resulted in TGI=43.7% (ΔAUC, p<0.001), when compared to vehicle. Compound A in combination with ibrutinib was found to have TGI of 68.8% (ΔAUC, p<0.001) and the combination was synergistic resulting in a statistically significant therapeutic advantage over single-agent treatments. Compound A in combination with bendamustine had TGI=78.6% (ΔAUC, p<0.001). This combination was also synergistic, demonstrating enhanced therapeutic potential over either single-agent treatment.

Compound A in combination with ibrutinib or bendamustine was found to be synergistic. All treatments and combinations were well tolerated. The greatest mean maximum BWL (1.7% on Day 5) was observed in the ibrutinib 6 mg/kg single-agent treatment group.

Test and control articles: The first test article used in this study was Compound A, formulated in 0.5% methylcellulose (MC). Compound A was prepared weekly and stored at room temperature (18° C. to 25° C.). The second test article used in this study was ibrutinib, formulated in 0.5% MC. Ibrutinib was prepared weekly and stored at room temperature (18° C. to 25° C.). The third test article used in this study was bendamustine formulated in 0.9% saline. Bendamustine was aliquoted and stored at approximately −20° C., and a fresh aliquot was prepared for each dose. The vehicle control was 0.5% MC. The dose volume for each vehicle or compound was 0.1 mL.

Experimental design: Female CB17 SCID Mus musculus mice (Taconic Farms, Inc; Cambridge City, Ind.; average weight at start of dosing was 19 g) were inoculated SC in the flank (cell suspension) with 4.0×10⁶ OCI-Ly10 cells in 50% Matrigel™. Tumor growth was monitored BIW using calipers and the mean tumor volume (MTV) was calculated using the formula (0.5×[length×width²]). When the MTV reached approximately 225 mm³, the animals were randomized into treatment groups (n=7/group). Mice were then dosed with vehicle (0.5% MC) or Compound A, ibrutinib, or bendamustine over a 21 day period as single agents or in combination.

Tumor growth and body weight were measured BIW. Tumor growth inhibition and body weight change were calculated on Day 21 of treatment. The first day of dosing was on Day 1 and dosing ended on Day 21. Measurements were obtained up to Day 48, but measurements after Day 21 were not included in this study. The mean maximum BWL was determined for each group using the mean body weight data from the treatment period, and the mean maximum percent body weight change was calculated on the basis of predose body weights. Percent TGI was calculated on Day 21. Inhibition of tumor growth was determined by calculating the percent TGI using the following formula: Percent TGI=(MTV of the control group−MTV of a treated group)÷MTV of the control group×100.

Antitumor activity was determined by statistical comparisons of tumor growth between treatment groups and vehicle, conducted using a linear mixed effects regression analysis on the ΔAUC. For further details see Example 2.

Statistical analysis: The differences in the tumor growth trends over time between the vehicle control and treatment groups were assessed using linear mixed effects regression models. These models take into account that each animal was measured at multiple time points. A model was fit for the comparison, and the areas under the tumor volume-time curve (AUCs) for control and treatment groups were calculated using the values predicted from the model. A statistically significant p value suggests that the trends over time for the 2 groups (vehicle and treatment) were different. A p value <0.05 was considered statistically significant. For further details see Example 2.

Days greater than 21 were excluded. All animals were included.

A combination score calculation was used to address the question of whether the effects of the combination treatments were synergistic, additive, subadditive, or antagonistic relative to the individual treatments. The effect was considered synergistic if the synergy score was less than 0, and additive if the synergy score wasn't statistically different from 0. If the synergy score was greater than 0, but the mean AUC for the combination was lower than the lowest mean AUC among the 2 single-agent treatments, then the combination was subadditive. If the synergy score was greater than the mean AUC for at least 1 of the single-agent treatments, then the combination was antagonistic.

Results and discussion: Mice were inoculated SC into the right flank with OCI-Ly10 human DLBCL cells and were treated QD with PO doses of vehicle, or Compound A. Ibrutinib was administered QD PO, and bendamustine was administered BIW IV as single agents or in combination with Compound A for 21 days. Effects on tumor growth were evaluated by measuring percent TGI. Tolerability was assessed by percent BWL, lethality and clinical signs of adverse treatment-related side effects. Body weights were measured BIW. The percentage TGI was determined on Day 21. Statistical comparisons of tumor growth between treatment groups and vehicle were conducted to assess antitumor activity using a linear mixed effects regression analysis on the ΔAUC. A p value less than 0.05 was considered statistically significant. Synergy analysis was conducted to evaluate the effects of combination treatment compared to single-agent treatment alone.

Compound A was administered QD PO at 60 mg/kg which resulted in TGI of 38.8% (ΔAUC, p<0.001). Ibrutinib was administered QD PO at 6 mg/kg and was found to have TGI=18.0% (ΔAUC, p<0.01), when compared to vehicle. Bendamustine, administered IV, on a BIW schedule (6 doses) at 1 mg/kg, resulted in TGI of 43.7% (ΔAUC, p<0.001).

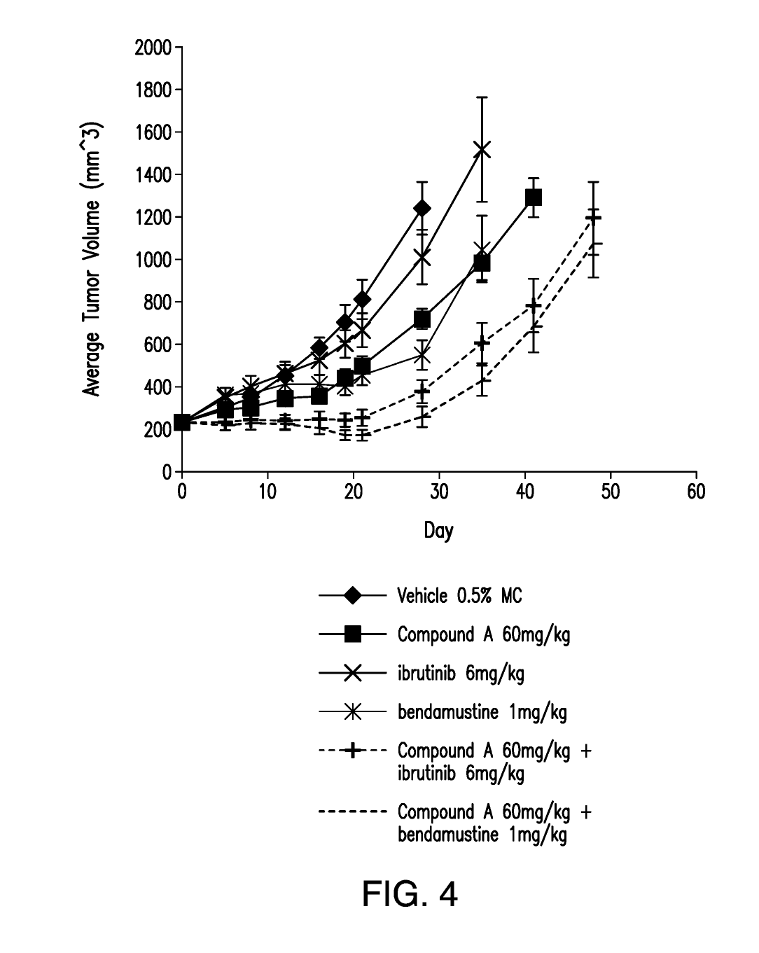

Compound A in combination with bendamustine resulted in TGI=78.6% (ΔAUC, p<0.001) when compared to vehicle. This combination was also synergistic, demonstrating enhanced therapeutic potential over either single-agent treatment. The MTV over time for each group is represented graphically in FIG. 4.

All of the treatments including the combination treatments were well tolerated by all of the animals. The ibrutinib and bendamustine single agent arms had less than a 2% average maximal BWL, and all of the other groups experienced an increase in body weight (see Table 11). These results suggest that ibrutinib or bendamustine can be combined with Compound A in mice without significantly increasing toxicity, as evidenced by acceptable changes in body weight in both studies.

The antitumor activity of Compound A, bendamustine, and ibrutinib alone or in combination against OCI-Ly10 DLBCL xenografts is summarized in Table 11 and graphically presented in FIG. 4. Results of the combination analysis are summarized in Table 12.

TABLE 11
Study design and findings for Compound A in OCI-Ly10 human tumor xenograft
model.

| Treatment | Dose | Administration | Sex/Number | | |
|---|---|---|---|---|---|
| Group | (mg/kg) | Method, Frequency | Per Group | Endpoints | Findings |
| vehicle | 0.0 | PO/QD (21 days) | F/7 | TGI^{a} | N/A |
| (0.5% MC) | | | | ΔAUC^{b} | N/A |
| | | | | Mean maximum % BWL^{c} | 0% |
| Compound A | 60 | PO/QD (21 days) | F/7 | TGI^{a} | 38.8% |
| | | | | ΔAUC^{b} | p < 0.001 |
| | | | | Mean maximum % BWL^{c} | 0% |
| ibrutinib | 6 | PO/QD (21 days) | F/7 | TGI^{a} | 18% |
| | | | | ΔAUC^{b} | p < 0.01 |
| | | | | Mean maximum % BWL^{c} | 1.7% (Day 5) |
| bendamustine | 1 | IV/BIW (6 doses, | F/7 | TGI^{a} | 43.7% |
| | | Days 1, 5, 8, 12, 15, | | ΔAUC^{b} | p < 0.001 |
| | | and 19) | | Mean maximum % BWL^{c} | 0.3% (Day 5) |
| Compound A + | 60; 6 | PO/QD (21 days);: | F/7 | TGI^{a} | 68.8% |
| ibrutinib | | PO/QD (21 days) | | ΔAUC^{b} | p < 0.001 |
| | | | | Mean maximum % BWL^{c} | 0% |
| | | | | Synergy analysis^{d} | Synergistic |
| Compound A + | 60; 1 | PO/QD (21 days); | F/7 | TGI^{a} | 78.6% |
| bendamustine | | IV/BIW (6 doses, | | ΔAUC^{b} | p < 0.001 |
| | | Days 1, 5, 8, 12, 15, | | Mean maximum % BWL^{c} | 0% |
| | | and 19) | | Synergy analysis^{d} | Synergistic |

ΔAUC = change in the area under the tumor volume-time curve;
BID = twice daily; BIW = twice weekly;
BWL = body weight loss;
F = female(s);
IV = intravenous(ly);
MC = methycellulose;
N/A = not applicable;
PO = oral(ly);
QD = once daily;
TGI = tumor growth inhibition.
^{a}TGI values were calculated on Day 21 of treatment.
^{b}ΔAUC = statistical analysis was performed with a linear mixed effects regression model. A p value of <0.05 was considered statistically significant.
^{c}Mean maximum percent BWL; 0% indicates, no BWL, animals in these groups gained weight.
^{d}Synergistic = effect was considered synergistic if the synergy score was less than 0, and additive if the synergy score wasn't statistically different from 0. If the synergy score was greater than 0, but the mean area under the tumor volume-time curve (AUC) for the combination was lower than the lowest mean AUC among the 2 single-agent treatments, then the combination was subadditive. If the synergy score was greater that the mean AUC for at least 1 of the single-agent treatments, then the combination was antagonistic.

TABLE 12
Combination comparisons (log-transformed) for Compound A
and ibrutinib or bendamustine in the OCI-Ly10 human tumor
xenograft model.

| Comparison | Score | SEM | P Value | Assessment |
|---|---|---|---|---|
| Compound A (60 mg/kg QD) + | −51.3 | 19.4 | <0.05 | Synergistic |
| ibrutinib (6 mg/kg QD) | | | | |
| Compound A (60 mg/kg QD) + | −48.1 | 18.8 | <0.05 | Synergistic |
| bendamustine (1 mg/kg BIW) | | | | |

BID = twice daily;
BIW = twice weekly;
QD = once daily;
SEM = standard error of the mean.
Note:
The effect was considered synergistic if the synergy score was less than 0 and additive if the synergy score wasn't statistically different from 0. If the synergy score was greater than 0, but the mean area under the tumor volume-time curve (AUC) for the combination was lower than the lowest mean AUC among the 2 single-agent treatments, then the combination was subadditive. If the synergy score was greater that the mean AUC for at least 1 of the single-agent treatments, then the combination was antagonistic.

Compound A in combination with ibrutinib or bendamustine was found to be synergistic. All treatments and combinations were well tolerated.

### Example 6

Antitumor activity of Compound A, bendamustine, and rituximab administered as single agents or combined in female SCID mice bearing OCI-Ly10 human lymphoma xenografts.

Tumor bearing mice were treated with 0.5% methylcellulose (e.g., the vehicle for Compound A), Compound A, bendamustine, and rituximab for three weeks. Effects on tumor growth were evaluated by measuring percent tumor growth inhibition (TGI) on Day 21 of the study. The change in the area under the tumor volume-versus-time curve (ΔAUC) was determined for treated groups versus control; a p value <0.05 was considered statistically significant. Tolerability was assessed by body weight loss (BWL) and lethality.

Compound A was administered PO at 30 or 60 mg/kg once daily for 21 days (QD×21). Bendamustine and rituximab was dosed intravenously (IV) at 1 mg/kg twice weekly (BIW) and once weekly (QW), respectively, for three weeks. The results of the pairwise comparisons showed that Compound A alone at 30 mg/kg had TGI value of 24.2% (ΔAUC, p=0.269) on Day 21. On the other hand, Compound A alone at 60 mg/kg and bendamustine or rituximab alone at 1 mg/kg had TGI values on Day 21 of 51.1% (ΔAUC, p<0.001), 49.7% (ΔAUC, p<0.001), and 33.4% (ΔAUC, p=0.047), respectively.

In animals treated with the pairwise combinations of Compound A, bendamustine, and rituximab, the resulting TGI values on Day 21 were 72.9% (ΔAUC, p<0.001), 34.7% (ΔAUC, p=0.002), and 83.2% (ΔAUC, p<0.001) for Compound A at 30 mg/kg plus bendamustine at 1 mg/kg, Compound A at 30 mg/kg plus rituximab at 1 mg/kg, and bendamustine at 1 mg/kg plus rituximab at 1 mg/kg, respectively. Compared to single agent therapy, the pairwise combinations of Compound A, bendamustine, and rituximab showed additive antitumor activities in the present study. Finally, in animals treated with Compound A at 30 mg/kg combined with bendamustine at 1 mg/kg as well as rituximab at 1 mg/kg, the TGI value on Day 21 was 70.0% (ΔAUC, p<0.001) for this triple combination group.

There was no mortality in the present study whether Compound A, bendamustine, and rituximab were given as single agents or combined. During the period from Day 0 to Day 21, no decrease in mean body weight was observed in vehicle (0.5% methylcellulose) control animals. The greatest % BWL detected in treatment groups was 1.1% in animals treated with Compound A at 30 mg/kg combined with bendamustine at 1 mg/kg as well as rituximab at 1 mg/kg.

Test and control articles: The first test article used in this study was Compound A formulated in 0.5% methylcellulose. Compound A solutions were prepared weekly and stored at room temperature (18 to 25° C.). The second test article used in this study was bendamustine formulated in water for injection (WFI). Bendamustine solution was prepared on Day 0 and stored at −20° C. until use. The third test article used in this study was rituximab for injection (100 mg/10 mL) formulated in 0.9% saline. Rituximab solution was prepared within 2 hours before dosing and stored on ice. Animals in the vehicle group were given 0.5% methylcellulose. The dose volume for PO and IV administration was 10 mL/kg body weight.

The dosing solutions preparations are summarized in Table 13.

TABLE 13
Compound A, bendamustine, and rituximab dosing solution preparations.

| | | Dose | | | | |
|---|---|---|---|---|---|---|
| Animals | Compound | (mg/kg) | Volume (mL/kg) | Conc. (mg/mL) | Route | Dosing Schedule |
| 8 | Compound A | 60 | 10 | 6 | PO | QD |
| 32 | Compound A | 30 | 10 | 3 | PO | QD |
| 32 | bendamustine | 1 | 10 | 0.1 | IV | BIW |
| 32 | rituximab | 1 | 10 | 0.1 | IV | QW |

The required volume for one week of dosing of Compound A was calculated as follows. For 60 mg/kg: 20 g (body weight)×8 (animals)×10 mL/kg/1000×7×1.5=16.8 mL. For 30 mg/kg: 20 g (body weight)×32 (animals)×10 mL/kg/1000×7×1.5=67.2 mL. Vehicle: 0.5% methylcellulose. The procedure for making Compound A at 6 mg/mL 15 mL solution was: (1) weigh 140.4 mg of Compound A powder; (2) add the powder in 15 mL of 0.5% methylcellulose; (3) sonicate for 5 minutes and then vortex for 30 minutes; (4) check the pH and the value shall be pH=3.5; (5) store at room temperature (18 to 25° C.) for one week. The procedure for making Compound A at 3 mg/mL 60 mL dosing solution was: (1) weigh 280.8 mg of Compound A powder; (2) add the powder in 60 mL of 0.5% methylcellulose; (3) sonicate for 5 minutes and then vortex for 30 minutes; (4) check the pH and the value shall be pH=3.5; (5) store at room temperature (18 to 25° C.) for one week. Vortex the solutions well before each dosing.

The required volume for one dosing of bendamustine was calculated as follows: 20 g×32 (animals)×10 mL/kg/1000×1.5% (50% extra)=9.6 mL. The procedure for making bendamustine (1 mg/mL) 10 mL solution was: (1) take one vial of bendamustine which contains 10 mg of active compound; (2) add 10 mL of water for injection (WFI); (3) dissolve all the powder to generate a solution of 1 mg/mL; (4) aliquot the solution to 10 tubes (1 mL/tube). The procedure for making bendamustine (0.1 mg/mL) 10 mL solution was: (1) take one tube prepared above that has 1 mL of bendamustine at 1 mg/mL; (2) add 9 mL of WFI; (3) mix well and store at −20 degree; (4) before dosing, take the vial out and thaw at room temperature (18 to 25° C.); (5) use for dosing within 2 hours.

The required volume for one dosing of rituximab was calculated as follows: 20 g (body weight)×32 (animals)×10 mL/kg/1000×1.5=9.6 mL. The rituximab stock solution was 10 mg/mL (100 mg/10 mL). The procedure for making rituximab (0.1 mg/mL) 10 mL solution was: (1) take 0.1 mL of rituximab stock solution into a centrifuge tube; (2) add 9.9 mL of 0.9% saline and mix manually; (3) store on ice and use within 2 hours.

Cell inoculation: OCI-Ly10 (human lymphoma cell line) tumor cell line was used. MAP and mycoplasma testing was negative. Preparation was Iscove's Modified Dulbecco's Medium (IMDM)+55 uM mercapoethanol+20% FBS. Passage—19. Vehicle was IMDM and cell number injected was 4×10⁶ cells per mouse (in 50% Matrigel™).

Dosing: Table 14 shows the planned dosing regimens for each treatment group used in the study. Vehicle (e.g., 0.5% methylcellulose) and Compound A at 30 or 60 mg/kg were administered PO (QD×21). Bendamustine at 1 mg/kg was given IV (BIW×3) on Day 1, 4, 8, 11, 15, and 18. Rituximab at 1 mg/kg was dosed IV (QW×3) on Day 1, 8, and 15. The first day of treatment was designated as Day 1 and the dosing lasted till Day 21.

Tumor volume and body weight measurements: Each animal (female SCID mice, Beijing HFK Bioscience Co., Ltd., 21.1-22.0 g at Day 0) was inoculated with 4×10⁶ OCI-Ly10 tumor cells (in 0.1 mL, 1:1 with Matrigel™) at the right flank. Body weight and the tumor growth were monitored twice weekly. Tumor size was measured to the nearest 0.1 mm using vernier caliper and applying the formula V=W²×L/2, where V=volume, W=width, and L=length for the tumor xenograft. Xenografts were allowed to grow until they reached an average size of approximately 210 mm³ after 17 days. Mice bearing the proper size xenograft were randomly assigned into one of the 9 groups shown in Table 14 and began to be treated with their assigned test materials, either vehicle (0.5% methylcellulose), Compound A, bendamustine, rituximab, or the combination of Compound A plus bendamustine and/or rituximab.

Tumor size and body weight were measured twice weekly beginning on the day of animal grouping (Day 0). The study was terminated following the last measurement on Day 21. The antitumor activity was determined by calculating the percent TGI on Day 21 using the following equation: Percent TGI=(MTV Vehicle group−MTV Treatment group)÷MTV Vehicle group×100. Statistical comparisons of tumor growth between treatment groups and vehicle were conducted using a linear mixed effects regression analysis on the ΔAUC.

Statistical tests: delta AUC. The differences in the tumor growth trends over time between the vehicle control and treatment groups were assessed using linear mixed effects regression models. These models take into account that each animal was measured at multiple time points. A model was fit for the comparison, and the areas under the tumor volume-versus-time curve (AUCs) for control and treatment groups were calculated using the values predicted from the model. A statistically significant p value suggests that the trends over time for the 2 groups (vehicle and treatment) were different. A p value <0.05 was considered statistically significant. Further details regarding pairwise comparisons are provided in Example 2.

Combination treatment effects: A combination score calculation was used to address the question of whether the effects of the combination treatments were synergistic, additive, subadditive, or antagonistic relative to the individual treatments. The effect was considered synergistic if the synergy score was less than 0 and additive if the synergy score wasn't statistically different from 0. If the synergy score was greater than 0, but the mean AUC for the combination was lower than the lowest mean AUC among the two single agent treatments, then the combination was subadditive. If the synergy score was greater than zero, and the mean AUC for the combination was greater than the mean AUC for at least one of the single agent treatments, then the combination was antagonistic.

Results and discussion: During the period from Day 0 to Day 21, no loss in mean body weight was observed in female SCID mice bearing OCI-Ly10 xenografts from the vehicle-treated group (0.5% m ethylcellulose, PO, QD×21). On Day 21, the mean body weight of the vehicle group increased 11.6% compared to Day 0 when the animals were grouped.

During the same period, no loss in mean body weight was detected in animals treated with Compound A alone at 60 mg/kg (PO, QD×21), Bendamustine alone at 1 mg/kg (IV, BIW×3), or with rituximab alone at 1 mg/kg (IV, QW×3). Meanwhile, the maximal mean % BWL was 1.0% (Day 4) in animals treated with Compound A alone at 30 mg/kg (PO, QD×21).

During the period from Day 0 to Day 21, no loss in mean body weight was detected in animals treated with the combination of rituximab at 1 mg/kg plus Compound A at 30 mg/kg or bendamustine at 1 mg/kg. Meanwhile, the maximal mean % BWL were 1.1% (Day 18) and 0.2% (Day 14), respectively, in animals treated with the combination of Compound A at 30 mg/kg plus bendamustine at 1 mg/kg with and without rituximab at 1 mg/kg.

No mortality occurred in any of the single agent or combination treatment groups. Changes in animal body weight following the administration of Compound A, bendamustine, and rituximab as single agents or combined are summarized in Table 14.

The PO administration of Compound A alone at 30 mg/kg (QD) had TGI value of 24.2% (ΔAUC=15.7, p=0.269) on Day 21. On the other hand, Compound A alone at 60 mg/kg (QD), bendamustine alone at 1 mg/kg (BIW), or rituximab alone at 1 mg/kg (QW) had TGI values on Day 21 of 51.1% (ΔAUC=34.3, p<0.001), 49.7% (ΔAUC=30.6, p<0.001), and 33.4% (ΔAUC=21.4, p=0.047), respectively.

When Compound A, bendamustine, and rituximab were administered in pairwise combination, the resulting TGI values on Day 21 were 72.9% (ΔAUC=51.6, p<0.001), 34.7% (ΔAUC=19.4, p=0.002), and 83.2% (ΔAUC=70.4, p<0.001) for Compound A at 30 mg/kg plus bendamustine at 1 mg/kg, Compound A at 30 mg/kg plus rituximab at 1 mg/kg, and bendamustine at 1 mg/kg plus rituximab at 1 mg/kg, respectively. The synergy analysis indicated that the interactions between Compound A, bendamustine, and rituximab were additive (Score=15.6, −6.5, and −19.4, p>0.05, see Table 15 for details). When Compound A at 30 mg/kg was administered in combination with both bendamustine at 1 mg/kg and rituximab at 1 mg/kg, the resulting TGI was 70.0% (ΔAUC=52.4, p<0.001) on Day 21.

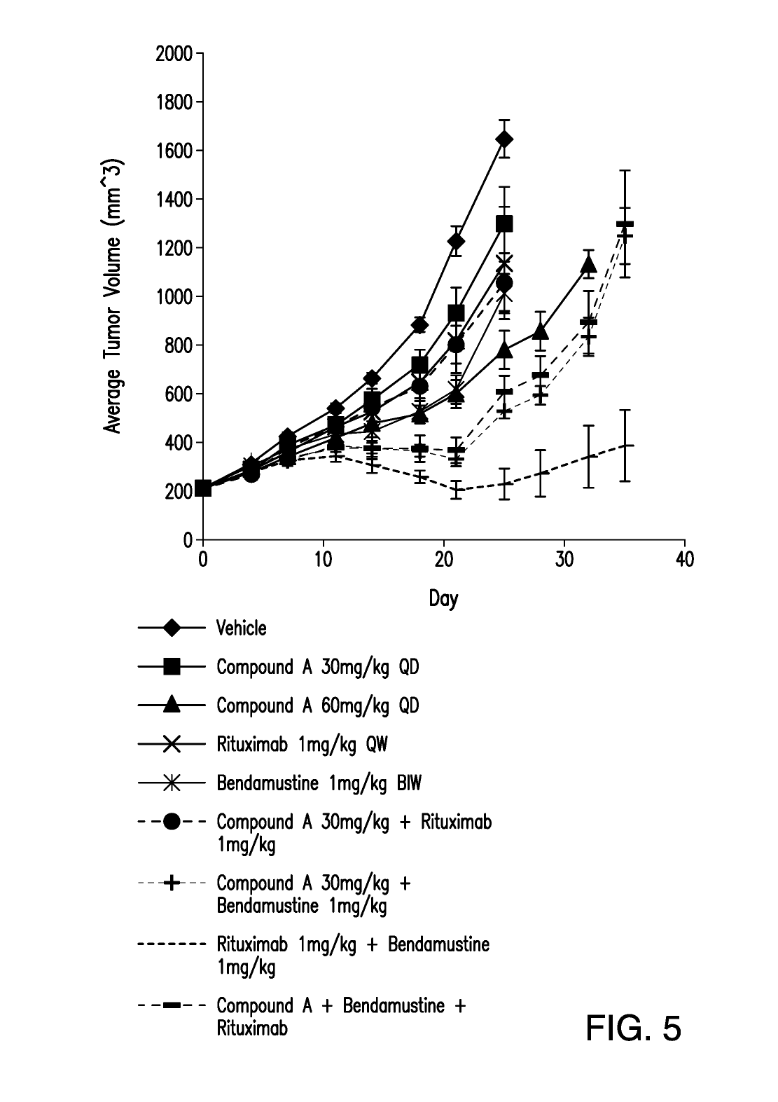

The antitumor activity of Compound A, bendamustine, and rituximab alone or combined against OCI-Ly10 xenografts is summarized in Table 14 and graphically presented in FIG. 5. The result of combination analysis is summarized in Table 15.

TABLE 14
Study design and findings for Compound A, bendamustine, and rituximab.

| Treatment | Dose | Administration | Sex/Number | | |
|---|---|---|---|---|---|
| Group | (mg/kg) | Method, Frequency | Per Group | Endpoints | Findings |
| 0.5% methyl- | N/A | PO/QD × 21, Day 1-21 | Female/8 | TGI^{b} | N/A |
| cellulose^{a} | | | | Mean maximum % BWL^{c} | 0% |
| Compound A | 30 | PO/QD × 21, Day 1-21 | Female/8 | TGI | 24.2% |
| | | | | ΔAUC^{d} | 15.7 (p = 0.269) |
| | | | | Maximum mean % BWL | 1.0% (Day 4) |
| Compound A | 60 | PO/QD × 21, Day 1-21 | Female/8 | TGI | 51.1% |
| | | | | ΔAUC | 34.3 (p < 0.001) |
| | | | | Maximum mean % BWL | 0% |
| rituximab | 1 | IV/QW × 3, Day 1,8, | Female/8 | TGI | 33.4% |
| | | 15 | | ΔAUC | 21.4 (p = 0.047) |
| | | | | Maximum mean % BWL | 0% |
| bendamustine | 1 | IV/BIW, Days 1, 4, 8, | Female/8 | TGI | 49.7% |
| | | 11, 15, 18 | | ΔAUC | 30.6 (p < 0.001) |
| | | | | Maximum mean % BWL | 0% |
| Compound A + | 30; 1 | PO/QD × 21, Day 1-21; | Female/8 | TGI | 34.7% |
| rituximab | | IV/QW × 3, Day 1, | | ΔAUC | 19.4 (p = 0.002) |
| | | 8, 15 | | Synergy analysis | Additive |
| | | | | Maximum mean % BWL | 0% |
| Compound A + | 30; 1 | PO/QD × 21, Day 1-21; | Female/8 | TGI | 72.9% |
| bendamustine | | IV/BIW, Days 1, 4, 8, | | ΔAUC | 51.6 (p < 0.001) |
| | | 11, 15, 18 | | Synergy analysis | Additive |
| | | | | Maximum mean % BWL | 0.2% (Day 14) |
| rituximab + | 1; 1 | IV/QW × 3, Day 1, 8, | Female/8 | TGI | 83.2% |
| bendamustine | | 15; IV/BIW, Days 1, | | ΔAUC | 70.4 (p < 0.001) |
| | | 4, 8, 11, 15, 18 | | Synergy analysis | Additive |
| | | | | Maximum mean % BWL | 0% |
| Compound A + | 30; 1; 1 | PO/QD × 21, Day 1-21; | Female/8 | TGI | 70.0% |
| rituximab + | | IV/QW × 3, Day 1, 8, | | ΔAUC | 52.4 (p < 0.001) |
| bendamustine | | 15; IV/BIW, Days 1, | | Maximum mean % BWL | 1.1% (Day 18) |
| | | 4, 8, 11, 15, 18 | | | |

ΔAUC = change in areas under the tumor volume-versus-time curves;
BIW = twice weekly;
BWL = body weight loss;
IV = intravenously;
NA = not applicable;
PO = orally;
QD = once daily;
QW = once weekly;
TGI = tumor growth inhibition.
^{a}Dose volume for PO or IV administration was 10 mL/kg body weight.
^{b}TGI values were calculated on Day 21 post treatment initiation.
^{c}Maximum mean percent BWL between Day 0 to Day 21.
^{d}ΔAUC = Statistical analysis was performed with a linear mixed effects regression model. A p value of <0.05 was considered statistically significant.

TABLE 15
Combination comparisons (log-transformed)
for Compound A, bendamustine, and rituximab.

| Comparison | Score | SEM | P-Value | Assess |
|---|---|---|---|---|
| Compound A (30 mg/kg, PO, QD) + | 15.6 | 16.0 | 0.342 | Additive |
| rituximab (1 mg/kg, IV, QW) | | | | |
| Compound A (30 mg/kg, PO, QD) + | −6.5 | 12.0 | 0.591 | Additive |
| bendamustine (1 mg/kg, IV, BIW) | | | | |
| rituximab (1 mg/kg, IV, QW) + | −19.4 | 14.7 | 0.206 | Additive |
| bendamustine (1 mg/kg, IV, BIW) | | | | |

BIW = twice weekly;
IV = intravenously;
PO = orally;
QD = once daily;
QW = once weekly;
SEM = standard error of the mean.

Synergistic analysis: p>0.05=additive; p<0.05 and score <0=synergistic; p<0.05, score >0, and the combination growth rate is lower than both the single agent growth rates=subadditive; p<0.05, score >0, and the combination growth rate is higher than at least 1 of the single agent growth rates=antagonistic. A p value <0.05 was considered statistically significant.

The pairwise interactions among Compound A and bendamustine or rituximab were additive. Meanwhile, female SCID mice bearing OCI-Ly10 xenografts could tolerate the treatments with Compound A, bendamustine, and rituximab well whether they were given as single agents or in combination.

### Example 7

Antitumor activity of Compound A and gemcitabine (administered orally and intraperitoneally, respectively) or their combination in female CB17 SCID mice bearing OCI-LY10 xenografts.

Compound A, gemcitabine, or vehicle were administered to female CB 17 SCID mice bearing OCI-LY 10 xenografts beginning on Day 1 for 21 days. Tumor growth inhibition was calculated on Day 21 of the study. The last measurement was taken on Day 42 of the study.

Compound A was administered once daily (QD), oral (PO), at 60 mg/kg for a total of 21 doses, which resulted in TGI=65.5% (ΔAUC, p≤0.001). Gemcitabine was administered every 3 days (Q3D), interperitoneally (IP) for a total of 4 doses, at 2.5 mg/kg, which resulted in TGI=52.6% (ΔAUC, p<0.001). Gemcitabine was administered every 3 days (Q3D), interperitoneally (IP), at 5 mg/kg for a total of 4 doses. This resulted in TGI=72.1% (ΔAUC, p<0.001).

Compound A at 60 mg/kg in combination with gemcitabine at 2.5 mg/kg achieved TGI=81.9% (ΔAUC, p<0.001). The combination was found to be additive. Compound A at 60 mg/kg in combination with gemcitabine at 5 mg/kg achieved TGI=90.6% (ΔAUC, p<0.001). This combination was also found to be additive. All of the groups were well tolerated with no body weight loss. The combination of Compound A and gemcitabine at 2.5 mg/kg was considered additive. The Compound A and gemcitabine at 5 mg/kg combination was also considered additive.

Experimental design: Female CB17 SCID mice (Taconic Biosciences; weight at treatment start was 20 g) were inoculated subcutaneously in the flank (cell suspension) with 4.0×106 OCI-Ly10 cells. Tumor volume was calculated using the formula V=W²×L/2, where V=volume, W=width and L=length of the tumor. When the mean tumor volume reached approximately 199 mm³, the animals were randomized into 6 treatment groups (n=8/group). Mice were then dosed with 0.5% methylcellulose or Compound A or gemcitabine over a 21 day period (see Table 16 for details). Tumor growth and body weight were measured twice per week. Tumor growth inhibition and body weight change were calculated on Day 21 of treatment.

Statistical analysis: The differences in the tumor growth trends over time between pairs of treatment groups were assessed using linear mixed effects regression models. These models account for the fact that each animal was measured at multiple time points. A separate model was fit for each comparison, and the areas under the curve (AUC) for each treatment group were calculated using the predicted values from the model. The percent decrease in AUC (dAUC) relative to the reference group was then calculated. A statistically significant P value suggests that the trends over time for the two treatment groups were different. Further details regarding pairwise comparisons are provided in the Example 2.

Drug combinations were assessed for synergy using observed AUC values. The change in AUC relative to the control was calculated for both single agent treatment groups as well as the combination group. The interaction between the two compounds was then assessed by comparing the change in AUC observed in the combination group to the sum of the changes observed in both single agents. The results can be divided into four categories: synergistic, additive, sub-additive, and antagonistic. Further details regarding combination analyses are provided in the Example 2.

Once a final analysis was selected, the tumor measurements observed on a date pre-specified by the researcher (typically the last day of treatment) were analyzed to assess tumor growth inhibition. For this analysis, a T/C ratio was calculated for each animal by dividing the tumor measurement for the given animal by the mean tumor measurement across all control animals. The T/C ratios across a treatment group were compared to the T/C ratios of the control group using a two-tailed Welch's t-test.

All P values <0.05 were called statistically significant. Days greater than 21 were excluded. All animals were included.

Results and discussion: Compound A, gemcitabine, or vehicle were administered to female CB 17 SCID mice bearing OCI-LY 10 xenografts beginning on Day 1 for 21 days. Tumor growth inhibition was calculated on Day 21 of the study. The last measurement was taken on Day 42 of the study.

Compound A was administered once daily (QD), oral (PO), at 60 mg/kg for a total of 21 doses, which resulted in TGI=65.5% (ΔAUC, p≤0.001). Gemcitabine was administered every 3 days (Q3D), interperitoneally (IP), at 2.5 mg/kg for a total of 4 doses, which resulted in TGI=52.6% (ΔAUC, p<0.001). Gemcitabine was administered every 3 days (Q3D), interperitoneally (IP), at 5 mg/kg for a total of 4 doses. This resulted in TGI=72.1% (ΔAUC, p<0.001). Compound A at 60 mg/kg in combination with gemcitabine at 2.5 mg/kg achieved TGI=81.9% (ΔAUC, p<0.001). The combination was found to be additive. Compound A at 60 mg/kg in combination with gemcitabine at 5 mg/kg achieved TGI=90.6% (ΔAUC, p<0.001). This combination was also found to be additive.

All of the groups were well tolerated with no body weight loss. No animals were removed from this study.

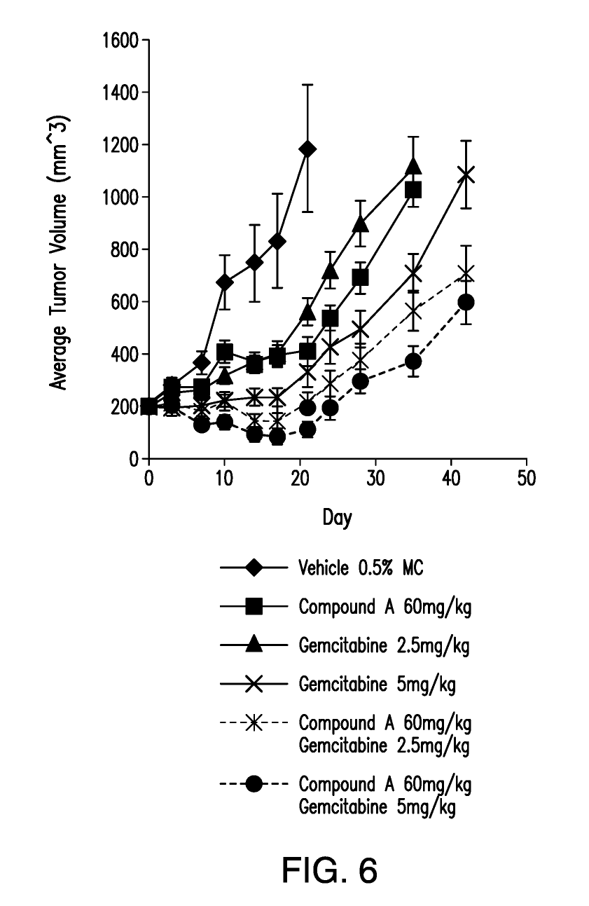

The antitumor activity of Compound A and gemcitabine alone or combined against OCI-Ly10 xenografts is summarized in Table 16 and graphically presented in FIG. 6. The result of combination analysis is summarized in Table 17.

TABLE 16
Tumor growth inhibition.

| | | | | % BW^{a} | Number | Mean Tumor | | | |
|---|---|---|---|---|---|---|---|---|---|
| | | | Route | Change | of | Volume | | | P- |
| | Dose | | Schedule | (Day | animals | (mm³) ± | | TGI^{d} | Value^{e} |
| Test Article | (mg/kg) | Sequencing | Duration | Maximum) | removed | SEM^{b} | T/C^{c} | (%) | (dAUC) |
| 0.5% methyl- | N/A | — | PO QD 21 | −0.1 (3) | 0 | 1,185.4 ± 243.6 | N/A | N/A | N/A |
| cellulose | | | | | | | | | |
| Compound A | 60.0 | — | PO QD 21 | 6.8 (21) | 0 | 409.4 ± 55.2 | 0.35 | 65.5 | 0.001 |
| gemcitabine | 2.5 | — | IP Q3D 21 | 7.5 (21) | 0 | 561.7 ± 52.5 | 0.47 | 52.6 | <0.001 |
| gemcitabine | 5.0 | — | IP Q3D 21 | −1.4 (3) | 0 | 331.3 ± 57.6 | 0.28 | 72.1 | <0.001 |
| Compound A, | 60.0; | Compound A// | PO QD 21; | 5.2 (21) | 0 | 214.3 ± 38.5 | 0.18 | 81.9 | <0.001 |
| gemcitabine | 2.5 | gemcitabine | IP Q3D 21 | | | | | | |
| Compound A, | 60.0; | Compound A// | PO QD 21; | −0.9 (3) | 0 | 111.5 ± 27 | 0.09 | 90.6 | <0.001 |
| gemcitabine | 5.0 | gemcitabine | IP Q3D 21 | | | | | | |

TGI and T/C values were calculated on Day 21 of treatment.
^{a}% Body weight change (Day of Maximum change within the treatment period).
^{b}Standard error of the mean.
^{c}Treatment over control.
^{d}TGI = 100 − [(treated average volume/control average volume) × 100].
^{e}The changes in the areas under the tumor volume versus time curves (ΔAUCs) were assessed using linear mixed effects regression models to compare treatment groups with vehicle. P-values <0.05 indicate the percent decrease in AUC (dAUC) relative to the reference group was statistically significant.

TABLE 17
Combination comparisons (log-transformed).

| Comparison | Score | SEM | P-Value | Assess |
|---|---|---|---|---|
| Compound A (60 mg/kg) + gemcitabine | −25.9 | 20 | 0.218 | Add. |
| (2.5 mg/kg) | | | | |
| Compound A (60 mg/kg) + gemcitabine | −42.2 | 25.9 | 0.139 | Add. |
| (5 mg/kg) | | | | |

Statistically significant negative synergy scores indicate a synergistic combination (“Syn.”). Statistically significant positive synergy scores indicate a sub-additive combination (“Sub-add.”) when the combination performs better (i.e. has a lower AUC) than the best performing single agent. Statistically significant positive synergy scores indicate an antagonistic combination (“Antag.”) when the combination performs worse than the best performing single agent. Scores that are not statistically significant are considered additive (“Add.”). P values less than 0.05 were considered statistically significant.

The combination of Compound A and gemcitabine at 2.5 mg/kg was considered additive. The Compound A and gemcitabine at 5 mg/kg combination was also considered additive.

### Example 8

Antitumor activity of Compound A and gemcitabine (administered orally and intraperitoneally, respectively) or their combination in female CB17 SCID mice bearing TMD8 DLBCL xenografts.

Compound A, gemcitabine, or vehicle were administered to female SCID mice bearing TMD8 DLBCL xenografts beginning on Day 1 for 14 days. Tumor growth inhibition was calculated on Day 14 of the study. The last measurement was taken on Day 40 of the study.

Compound A was administered once daily (QD), oral (PO), at 60 mg/kg for a total of 14 doses, which resulted in TGI=−6.8% (ΔAUC, p>0.05). Gemcitabine was administered once every three days for a total of 4 doses (Q3D×4), intraperitoneally (IP), at 5 mg/kg which resulted in TGI=67.1% (ΔAUC, p<0.001). Compound A in combination with gemcitabine achieved TGI=96.7% (ΔAUC, p<0.001). This combination was found to be synergistic, and 5 of 8 animals had no measurable tumor on Day 14.

All of the groups were well tolerated with less than a 1% BW loss in the gemcitabine single agent and combination group. All of the other groups experienced an increase in body weight. Compound A did not have any activity as a single agent in this study at the doses and schedules tested. Compound A and gemcitabine in combination resulted in antitumor activity that was found to be synergistic.

Experimental design: Female CB17 SCID mice (Taconic Biosciences; weight at treatment start was 19 g) were inoculated subcutaneously in the flank (cell suspension) with 5.0×10⁶ TMD8 cells. Tumor growth was monitored with vernier calipers. Tumor volume was calculated using the formula V=W²×L/2, where V=volume, W=width and L=length of the tumor. When the mean tumor volume reached approximately 215 mm³, the animals were randomized into treatment groups (n=8/group). Mice were then dosed with 0.5% methylcellulose or Compound A or gemcitabine over a 14 day period (see Table 18 for details). Tumor growth and body weight were measured twice per week. Tumor growth inhibition and body weight change were calculated on Day 14 of treatment.

Statistical analysis: The differences in the tumor growth trends over time between pairs of treatment groups were assessed using linear mixed effects regression models. These models account for the fact that each animal was measured at multiple time points. A separate model was fit for each comparison, and the areas under the curve (AUC) for each treatment group were calculated using the predicted values from the model. The percent decrease in AUC (dAUC) relative to the reference group was then calculated. A statistically significant P value suggests that the trends over time for the two treatment groups were different. Further details regarding pairwise comparisons are provided in the Example 2.

Drug combinations were assessed for synergy using observed AUC values. The change in AUC relative to the control was calculated for both single agent treatment groups as well as the combination group. The interaction between the two compounds was then assessed by comparing the change in AUC observed in the combination group to the sum of the changes observed in both single agents. The results can be divided into four categories: synergistic, additive, sub-additive, and antagonistic. Further details regarding combination analysis are provided in the Example 2.

Once a final analysis was selected, the tumor measurements observed on a date pre-specified by the researcher (typically the last day of treatment) were analyzed to assess tumor growth inhibition. For this analysis, a T/C ratio was calculated for each animal by dividing the tumor measurement for the given animal by the mean tumor measurement across all control animals. The T/C ratios across a treatment group were compared to the T/C ratios of the control group using a two-tailed Welch's t-test.

All P values <0.05 were called statistically significant in this Example. Days greater than 14 were excluded. All animals were included.

Results and discussion: Compound A, gemcitabine, or vehicle were administered to female SCID mice bearing TMD8 DLBCL xenografts beginning on Day 1 for 14 days. Tumor growth inhibition was calculated on Day 14 of the study. The last measurement was taken on Day 40 of the study.

Compound A was administered once daily (QD), oral (PO), at 60 mg/kg for a total of 14 doses, which resulted in TGI=−6.8% (ΔAUC, p>0.05). Gemcitabine was administered once every three days for a total of 4 doses (Q3D×4), intraperitoneally (IP), at 5 mg/kg which resulted in TGI=67.1% (ΔAUC, p<0.001). Compound A in combination with gemcitabine achieved TGI=96.7% (ΔAUC, p<0.001). This combination was found to be synergistic, and 5 of 8 animals had no measurable tumor on Day 14.

All of the groups were well tolerated with less than a 1% BW loss in the gemcitabine single agent and combination group. All of the other groups experienced an increase in body weight. No animals were removed from this study.

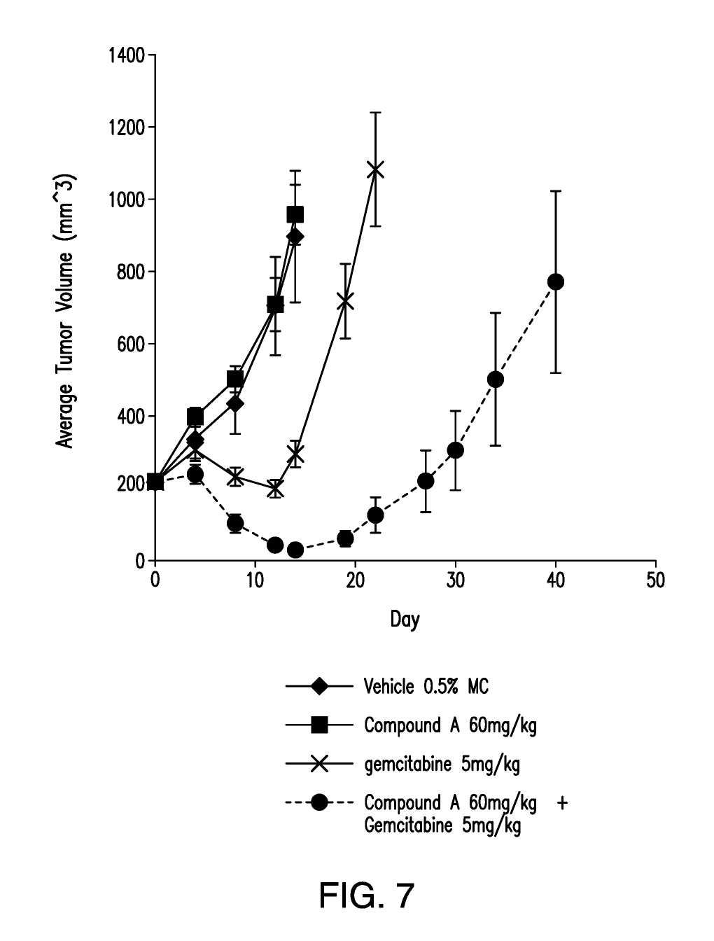

The antitumor activity of Compound A and gemcitabine alone or combined against TMD8 DLBCL xenografts is summarized in Table 18 and graphically presented in FIG. 7. The result of combination analysis is summarized in Table 19.

TABLE 18
Tumor growth inhibition.

| | | | | | | Mean | | | |
|---|---|---|---|---|---|---|---|---|---|
| | | | | % BW^{a} | Number | Tumor | | | |
| | | | Route | Change | of | Volume | | | P- |
| | Dose | | Schedule | (Day | animals | (mm³) ± | | TGI^{d} | Value^{e} |
| Test Article | (mg/kg) | Sequencing | Duration | Maximum) | removed | SEM^{b} | T/C^{c} | (%) | (dAUC) |
| 0.5% methyl- | N/A | — | PO QD 14 | 7.1 (14) | 0 | 896.5 ± 181.6 | N/A | N/A | N/A |
| cellulose | | | | | | | | | |
| Compound A | 60.0 | — | PO QD 14 | 6.4 (12) | 0 | 957.2 ± 82.2 | 1.07 | −6.8 | 0.062 |
| gemcitabine | 5.0 | — | IP Q3D × 4 | −0.1 (4) | 0 | 294.7 ± 36.5 | 0.33 | 67.1 | <0.001 |
| | | | 14 | | | | | | |
| Compound A, | 60.0; | Compound A// | PO QD 14; | −0.8 (4) | 0 | 29.4 ± 15.1 | 0.03 | 96.7 | <0.001 |
| gemcitabine | 5.0 | gemcitabine | IP Q3D × 4 | | | | | | |
| | | | 14 | | | | | | |

TGI and T/C values were calculated on Day 14 of treatment.
^{a}% Body weight change (Day of Maximum change within the treatment period).
^{b}Standard error of the mean.
^{c}Treatment over control.
^{d}TGI = 100 − [(treated average volume/control average volume) × 100].
^{e}The changes in the areas under the tumor volume versus time curves (ΔAUCs) were assessed using linear mixed effects regression models to compare treatment groups with vehicle. P-values <0.05 indicate the percent decrease in AUC (dAUC) relative to the reference group was statistically significant.

TABLE 19
Combination comparisons (log-transformed).

| Comparison | Score | SEM | P-Value | Assess |
|---|---|---|---|---|
| Compound A (60 mg/kg) + | −275.7 | 69.7 | 0.005 | Syn. |
| gemcitabine (5 mg/kg) | | | | |

Statistically significant negative synergy scores indicate a synergistic combination (“Syn.”). Statistically significant positive synergy scores indicate a sub-additive combination (“Sub-add.”) when the combination performs better (i.e. has a lower AUC) than the best performing single agent. Statistically significant positive synergy scores indicate an antagonistic combination (“Antag.”) when the combination performs worse than the best performing single agent. Scores that are not statistically significant are considered additive (“Add.”). P values less than 0.05 were considered statistically significant.

Compound A and gemcitabine combination was found to be synergistic.

### Example 9

Antitumor activity of Compound A, ABT-199, and gemcitabine (administered orally, intraperitoneally) or combination of Compound A and ABT-199 or gemcitabine in female CB17 SCID mice bearing TMD8 DLBCL xenografts.

Compound A, ABT-199, gemcitabine, or vehicle were administered to female SCID mice bearing TMD8 DLBCL xenografts beginning on Day 1 for 14 days. Tumor growth inhibition was calculated on Day 14 of the study. The last measurement was taken on Day 29 of the study.

Compound A was administered once daily (QD), oral (PO), at 60 mg/kg for a total of 14 doses, which resulted in TGI=25.7% (ΔAUC, p<0.05). ABT-199 was administered QD, PO, at 25 mg/kg for a total of 14 doses, which achieved TGI=29.1% (ΔAUC, p<0.05). Gemcitabine was administered once every three days for a total of 4 doses (Q3D×4), intraperitoneally (IP), at 5 mg/kg which resulted in TGI=70.3% (ΔAUC, p<0.001). The combination of Compound A with ABT-199 was found to be additive, with TGI=36.0% (ΔAUC, p<0.001). The combination of Compound A with gemcitabine was found to be synergistic, with TGI=100% (ΔAUC, p<0.001). The animals in this group did not have any palpable tumors at the end of treatment on Day 14. Following the cessation of treatment, the tumors did re-form.

All single agent and combination treatments were well tolerated, with a less than 1% maximal average body weight loss in the ABT-199 and gemcitabine single agent treatment groups, in comparison to 8.6% body weight gain of the control treatment group. The combination of Compound A with ABT-199 was additive. The combination of Compound A with gemcitabine was found to be synergistic in this study, with complete tumor regressions at the end of treatment.

Experimental design: Female CB17 SCID mice (Taconic Biosciences; weight at treatment start was about 19 g) were inoculated subcutaneously in the flank (cell suspension) with 5.0×10⁶ TMD8 cells. Tumor growth was monitored with vernier calipers. Tumor volume was calculated using the formula V=W²×L/2, where V=volume, W=width and L=length of the tumor. When the mean tumor volume reached approximately 245 mm³, the animals were randomized into 6 treatment groups (n=8/group). Mice were then dosed with 0.5% methylcellulose or Compound A or ABT-199 or gemcitabine over a 14 day period (see Table 20 for details). Tumor growth and body weight were measured twice per week. Tumor growth inhibition and body weight change were calculated on Day 14 of treatment.

Statistical analysis: The differences in the tumor growth trends over time between pairs of treatment groups were assessed using linear mixed effects regression models. These models account for the fact that each animal was measured at multiple time points. A separate model was fit for each comparison, and the areas under the curve (AUC) for each treatment group were calculated using the predicted values from the model. The percent decrease in AUC (dAUC) relative to the reference group was then calculated. A statistically significant P value suggests that the trends over time for the two treatment groups were different. Further details regarding pairwise comparisons are provided in the Example 2.

Drug combinations were assessed for synergy using observed AUC values. The change in AUC relative to the control was calculated for both single agent treatment groups as well as the combination group. The interaction between the two compounds was then assessed by comparing the change in AUC observed in the combination group to the sum of the changes observed in both single agents. The results can be divided into four categories: synergistic, additive, sub-additive, and antagonistic. Further details regarding combination analyses are provided in the Example 2.

Once a final analysis was selected, the tumor measurements observed on a date pre-specified by the researcher (typically the last day of treatment) were analyzed to assess tumor growth inhibition. For this analysis, a T/C ratio was calculated for each animal by dividing the tumor measurement for the given animal by the mean tumor measurement across all control animals. The T/C ratios across a treatment group were compared to the T/C ratios of the control group using a two-tailed Welch's t-test.

All P values <0.05 were called statistically significant in this Example. Days greater than 14 were excluded. All animals were included.

Results and discussion: Compound A, ABT-199, gemcitabine, or vehicle were administered to female SCID mice bearing TMD8 DLBCL xenografts beginning on Day 1 for 14 days. Tumor growth inhibition was calculated on Day 14 of the study. The last measurement was taken on Day 29 of the study.

Compound A was administered once daily (QD), oral (PO), at 60 mg/kg for a total of 14 doses, which resulted in TGI=25.7% (ΔAUC, p<0.05). ABT-199 was administered QD, PO, at 25 mg/kg for a total of 14 doses, which achieved TGI=29.1% (ΔAUC, p<0.05). Gemcitabine was administered once every three days for a total of 4 doses (Q3D×4), intraperitoneally (IP), at 5 mg/kg which resulted in TGI=70.3% (ΔAUC, p<0.001). The combination of Compound A with ABT-199 was found to be additive, with TGI=36% (ΔAUC, p<0.001). The combination of Compound A with gemcitabine was found to be synergistic, with TGI=100% (ΔAUC, p<0.001). The animals in this group did not have any palpable tumors at the end of treatment on Day 14. Following the cessation of treatment, the tumors did re-form.

All single agent and combination treatments were well tolerated, with a less than 1% maximal average body weight loss in the ABT-199 and gemcitabine single agent treatment groups, in comparison to 8.6% body weight gain of the control treatment group. No animals were removed during the treatment period.

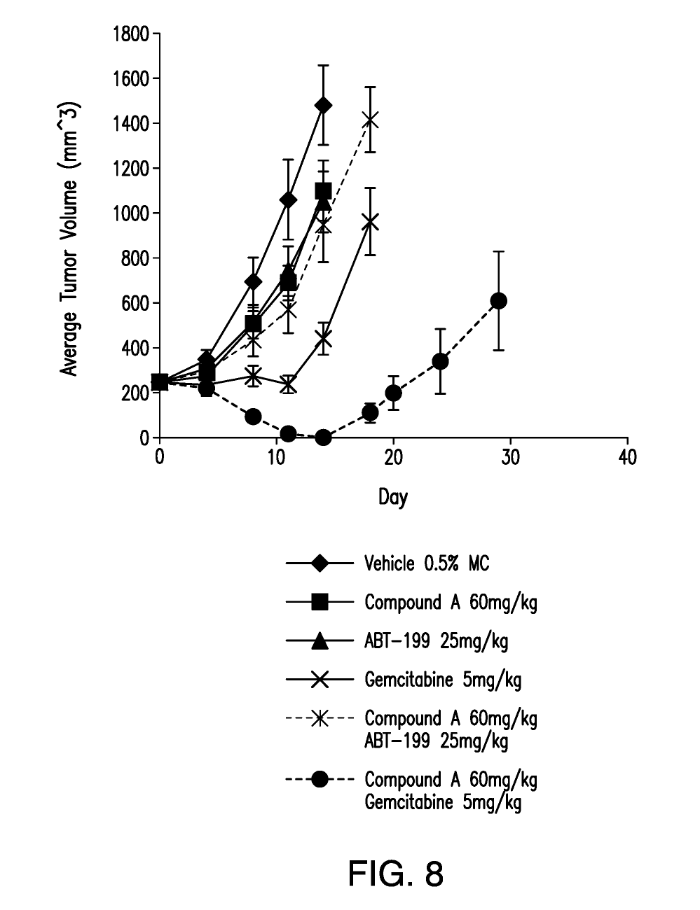

The antitumor activity of Compound A and gemcitabine alone or combined against OCI-Ly10 xenografts is summarized in Table 20 and graphically presented in FIG. 8. The result of combination analysis is summarized in Table 21.

TABLE 20
Tumor growth inhibition.

| | | | | % BW^{a} | Number | Mean Tumor | | | |
|---|---|---|---|---|---|---|---|---|---|
| | | | Route | Change | of | Volume | | | P- |
| | Dose | | Schedule | (Day | animals | (mm³) ± | | TGI^{d} | Value^{e} |
| Test Article | (mg/kg) | Sequencing | Duration | Maximum) | removed | SEM^{b} | T/C^{c} | (%) | (dAUC) |
| 0.5% | N/A | | PO QD 14 | 8.6 (14) | 0 | 1,480.2 ± 177.1 | N/A | N/A | N/A |
| methyl- | | | | | | | | | |
| cellulose | | | | | | | | | |
| Compound A | 60.0 | | PO QD 14 | 7.9 (14) | 0 | 1,100.2 ± 134.5 | 0.74 | 25.7 | 0.01 |
| ABT-199 | 25.0 | | PO QD 14 | −0.4 (11) | 0 | 1,048.9 ± 136.1 | 0.71 | 29.1 | 0.019 |
| gemcitabine | 5.0 | | IP Q3D × 4 | −0.2 (11) | 0 | 440.2 ± 70.9 | 0.30 | 70.3 | <0.001 |
| | | | 14 | | | | | | |
| Compound A: | 60.0; | Compound A// | PO QD 14; | 4.6 (14) | 0 | 948 ± 166.8 | 0.64 | 36.0 | <0.001 |
| ABT-199 | 25.0 | ABT-199 | PO QD 14 | | | | | | |
| Compound A; | 60.0; | Compound A// | PO QD 14; | 3.9 (8) | 0 | 0 ± 0 | 0.00 | 100.0 | <0.001 |
| gemcitabine | 5.0 | gemcitabine | IP Q3D × 4 | | | | | | |
| | | | 14 | | | | | | |

TGI and T/C values were calculated on Day 14 of treatment.
^{a}% Body weight change (Day of Maximum change within the treatment period).
^{b}Standard error of the mean.
^{c}Treatment over control.
^{d}TGI = 100 − [(treated average volume/control average volume) × 100].
^{e}The changes in the areas under the tumor volume versus time curves (ΔAUCs) were assessed using linear mixed effects regression models to compare treatment groups with vehicle. P-values <0.05 indicate the percent decrease in AUC (dAUC) relative to the reference group was statistically significant.

TABLE 21
Combination Comparisons (log-transformed).

| Comparison | Score | SEM | P-Value | Assess |
|---|---|---|---|---|
| Compound A (60 mg/kg) + | 16.3 | 18.6 | 0.392 | Add. |
| ABT-199 (25 mg/kg) | | | | |
| Compound A (60 mg/kg) + | −194.3 | 31.7 | <0.001 | Syn. |
| gemcitabine (5 mg/kg) | | | | |

Statistically significant negative synergy scores indicate a synergistic combination (“Syn.”). Statistically significant positive synergy scores indicate a sub-additive combination (“Sub-add.”) when the combination performs better (i.e. has a lower AUC) than the best performing single agent. Statistically significant positive synergy scores indicate an antagonistic combination (“Antag.”) when the combination performs worse than the best performing single agent. Scores that are not statistically significant are considered additive (“Add.”). P values less than 0.05 were considered statistically significant.

The combination of Compound A with ABT-199 was additive. The combination of Compound A with gemcitabine was found to be synergistic in this study, with complete tumor regressions at the end of treatment, confirming the results of a previous study.

### Example 10

Antitumor activity of Compound A and lenalidomide administered as single agents or combined in female SCID mice bearing OCI-Ly10 human lymphoma xenografts.

Tumor bearing mice were treated with 0.5% methylcellulose (e.g., the vehicle for Compound A), Compound A and lenalidomide for three weeks. Effects on tumor growth were evaluated by measuring percent tumor growth inhibition (TGI) on Day 21 of the study. The change in the area under the tumor volume-versus-time curve (ΔAUC) was determined for treated groups versus control; a p value <0.05 was considered statistically significant. Synergistic analysis was classified into four different categories based on synergistic score: synergistic, sub-additive, additive and antagonistic. Tolerability was assessed by percentage body weight loss (% BWL) and lethality.

Compound A at 60 mg/kg and lenalidomide at 10 mg/kg were administered orally (PO) once daily for 21 days (QD×21). The results of the pairwise comparisons showed Compound A alone at 60 mg/kg or lenalidomide alone at 10 mg/kg had TGI values on Day 21 of 43.6% (ΔAUC, p<0.001) and 21.1% (ΔAUC, p=0.014), respectively. The combination of Compound A plus lenalidomide had TGI value on Day 21 of 71.4% (ΔAUC, p<0.001). Compared to single agent therapy, the combination of Compound A plus lenalidomide at 10 mg/kg was additive (Score=6.8, p>0.05).

There was no mortality in the present study whether Compound A and lenalidomide were given as single agents or combined. During the period from Day 0 to Day 21, no decrease in mean body weight was observed in vehicle (0.5% methylcellulose) control animals.

Test and control articles: Compound A was formulated in 0.5% methylcellulose. Compound A solution was prepared weekly and stored at room temperature (18 to 25° C.). Lenalidomide (free base) was formulated in 1×phosphate-buffered saline (PBS). Lenalidomide solution was prepared once for the entire study and freezed at −20° C. before daily using.

Animals in the vehicle group were given 0.5% methylcellulose. The dose volume for Compound A administration was 10 mL/kg body weight. The dose volume for lenalidomide administration was 5 mL/kg body weight. The dosing solutions preparations are summarized in Table 22.

TABLE 22
Compound A and lenalidomide dosing solution preparations.

| | Dose | Volume | | | Dosing |
|---|---|---|---|---|---|
| Compound | (mg/kg) | (mL/kg) | Conc. (mg/mL) | Route | Schedule |
| Compound A | 60 | 10 | 6 | PO | QD |
| lenalidomide | 10 | 5 | 2 | PO | QD |

The procedure for preparation of Compound A at 6 mg/mL 60 mL solution was: (1) weigh 561.6 mg of Compound A powder; (2) add the powder in 60 mL of 0.5% methylcellulose; (3) sonicate for 5 minutes and then vortex for 30 minutes; (4) check the pH and the value shall be pH=3.5; (4) store at room temperature (18 to 25° C.) for one week. Vortex the solutions well before each dosing.

The required volume for 21 dosings of lenalidomide was calculated as follows: 20 g (body weight)×16 (animals)×5 mL/kg/1000×21×1.5=50.4 mL. Lenalidomide was stored in the freezer when not in use. It was allowed to warm to ambient temperature before opening. Vehicle: 1×PBS. The procedure for preparation of lenalidomide at 2 mg/mL 50 mL solution was: (1) weigh 100 mg of lenalidomide powder; (2) add 43.3 mL of 1×PBS; (3) add 1N HCl (about 2 mL) to pH ˜2, resulting a clear, colorless solution; (4) adjust pH to ˜6.5 by the addition of 1N NaOH; (5) add 1×PBS to a total volume of 50 mL; (6) aliquot about 2.2 mL solution per vial to 21 vials; (7) freeze the vials at −20 degree; (8) take one vial each day and thaw to room temperature (18 to 25° C.) for dosing.

Cell inoculation: OCI-Ly10 (Human Lymphoma Cell Line) tumor cell line was used. MAP and mycoplasma testing was negative. Preparation was IMDM+55 uM mercapoethanol+20% FBS. Passage—17. Vehicle was IMDM and cell number injected was 4×10⁶ cells per mouse (in 50% Matrigel™).

Dosing regimen: Table 23 shows the planned dosing regimens for each treatment group used in the study. Vehicle (e.g., 0.5% methylcellulose), Compound A at 60 mg/kg, and lenalidomide at 10 mg/kg were administered PO (QD×21). The first day of treatment was designated as Day 1 and the dosing lasted till Day 21.

Tumor volume and body weight measurements: Each animal (female SCID mice; Beijing HFK Bioscience Co., Ltd.; group average weight at Day 0 was 20.3-21.2 g; acclimation period >3 days) was inoculated with 4×10⁶ OCI-Ly10 tumor cells (in 0.1 mL, 1:1 with Matrigel™) at the right flank. Body weight and the tumor growth were monitored twice weekly. Tumor size was measured to the nearest 0.1 mm using vernier caliper and applying the formula V=W²×L/2, where V=volume, W=width, and L=length for the tumor xenograft. Xenografts were allowed to grow until they reached an average size of approximately 200 mm³ after 14 days. Mice bearing the proper size xenograft were randomly assigned into one of the groups shown in Table 23 and began to be treated with their assigned test materials, either vehicle (0.5% methylcellulose), Compound A, lenalidomide, or the combination of Compound A plus lenalidomide.

Tumor size and body weight were measured twice weekly beginning on the day of animal grouping (Day 0). The study was terminated following the last measurement on Day 42. The antitumor activity was determined by calculating the percent TGI on Day 21 using the following equation: Percent TGI=(MTV_{Vehicle group}−MTV_{Treatment group})÷MTV Vehicle group×100. Statistical comparisons of tumor growth between treatment groups and vehicle were conducted using a linear mixed effects regression analysis on the ΔAUC.

Statistical tests: The differences in the tumor growth trends over time between the vehicle control and treatment groups were assessed using linear mixed effects regression models. These models take into account that each animal was measured at multiple time points. A model was fit for the comparison, and the areas under the tumor volume-versus-time curve (AUCs) for control and treatment groups were calculated using the values predicted from the model. A statistically significant p value suggests that the trends over time for the 2 groups (vehicle and treatment) were different. A p value <0.05 was considered statistically significant in this Example. Further details regarding pair-wise comparisons are provided in Example 2.

A combination score calculation was used to address the question of whether the effects of the combination treatments were synergistic, additive, subadditive, or antagonistic relative to the individual treatments. The effect was considered synergistic if the synergy score was less than 0 and additive if the synergy score wasn't statistically different from 0. If the synergy score was greater than 0, but the mean AUC for the combination was lower than the lowest mean AUC among the two single agent treatments, then the combination was subadditive. If the synergy score was greater than zero, and the mean AUC for the combination was greater than the mean AUC for at least one of the single agent treatments, then the combination was antagonistic.

Results and discussion: During the period from Day 0 to Day 21, no loss in mean body weight was observed in female SCID mice bearing OCI-Ly10 xenografts from the vehicle-treated group (0.5% methylcellulose, PO, QD×21). On Day 21, the mean body weight of the vehicle group increased 8.8% compared to Day 0 when the animals were grouped.

During the same period, no loss in mean body weight was detected in animals treated with Compound A alone at 60 mg/kg (PO, QD×21) or with lenalidomide alone at 10 mg/kg (PO, QD×21). The maximal mean % BWL was 0.4% (Day 4) in animals treated with the combination of Compound A at 60 mg/kg plus lenalidomide at 10 mg/kg. There was no mortality in any of the single agent or combination treatment groups. Changes in animal body weight following the administration of Compound A and lenalidomide as single agents or combined are summarized in Table 23.

The synergy analysis indicated that the interaction between Compound A and lenalidomide at 10 mg/kg was additive (Score=6.8, p=0.395).

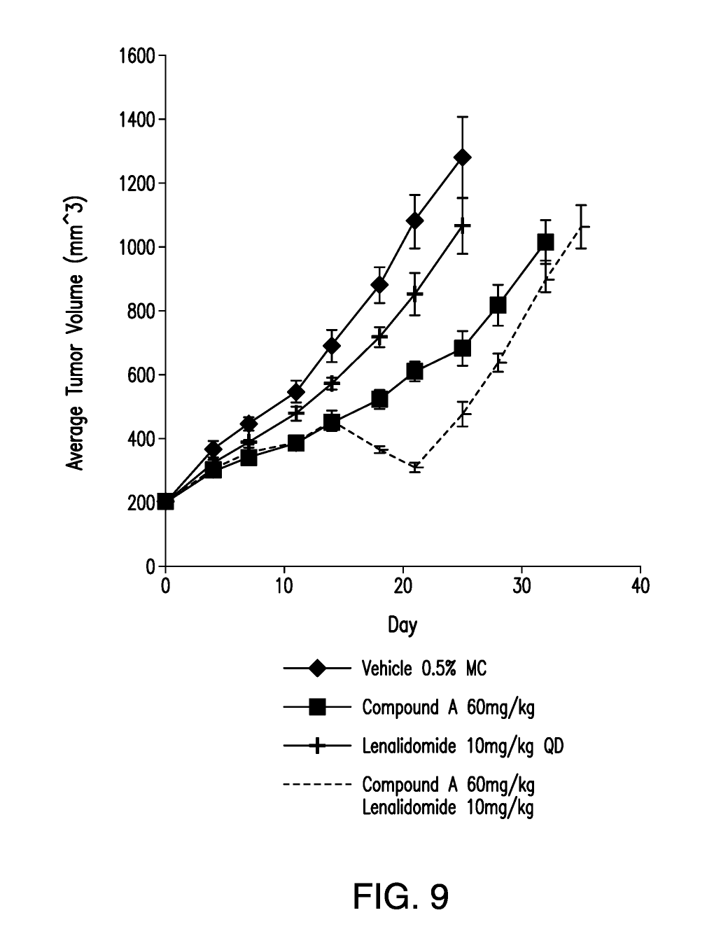

The antitumor activity of Compound A and lenalidomide alone or combined against OCI-Ly10 xenografts is summarized in Table 23 and graphically presented in FIG. 9. The result of combination analysis is summarized in Table 24.

TABLE 23
Study design and findings for Compound A and lenalidomide.

| | | Method of | | | |
|---|---|---|---|---|---|
| | Dose | Administration, | Sex/Number | | |
| Treatment Group | (mg/kg) | Frequency | Per Group | Endpoints | Findings |
| 0.5% methyl- | NA | PO/QD × 21 Day 1-21 | Female/8 | TGI^{b} | N/A |
| cellulose^{a} | | | | Mean | 0% |
| | | | | maximum % BWL^{c} | |
| Compound A | 60 | PO/QD × 21 Day 1-21 | Female/8 | TGI | 43.6% |
| | | | | ΔAUC^{d} | 34.7 (p < 0.001) |
| | | | | Mean maximum % BWL | 0% |
| lenalidomide | 10 | PO/QD × 21 Day 1-21 | Female/8 | TGI | 21.1% |
| | | | | ΔAUC | 15.2 (p = 0.014) |
| | | | | Mean maximum % BWL | 0% |
| Compound A + | 60 + 10 | PO/QD × 21 Day 1-21 | Female/8 | TGI | 71.4% |
| lenalidomide | | PO/QD × 21 Day 1-21 | | ΔAUC | 44.1 (p < 0.001) |
| | | | | Synergy analysis | Additive |
| | | | | Mean maximum % BWL | 0.4% (Day 4) |

ΔAUC = change in areas under the tumor volume-versus-time curves;
BWL = body weight loss;
IP = intraperitoneally;
NA = not applicable;
PO = orally;
QD = daily;
QW = once weekly;
TGI = tumor growth inhibition.
^{a}Dose volume for 0.5% methylcellulose and Compound A administration was 10 mL/kg body weight. Dose volume for lenalidomide administration was 10 mL/kg body weight.
^{b}TGI values were calculated on Day 21 post treatment initiation.
^{c}Maximum mean percent BWL between Day 0 to Day 21.
^{d}ΔAUC = Statistical analysis was performed with a linear mixed effects regression model. A p value of <0.05 was considered statistically significant.

TABLE 24
Combination comparisons (log-transformed) for Compound A and
lenalidomide.

| Comparison | Score | SEM | P-Value | Assess |
|---|---|---|---|---|
| Compound A (60 mg/kg, PO, QD) + | 6.8 | 7.9 | 0.395 | Additive |
| lenalidomide (10 mg/kg, PO, QD) | | | | |

IV = intraperitoneally;
PO = orally;
QD = daily;
QW = once weekly;
SEM = standard error of the mean.

Synergistic analysis: p>0.05=additive; p<0.05 and score <0=synergistic; p<0.05, score >0, and the combination growth rate is lower than both the single agent growth rates=subadditive; p<0.05, score >0, and the combination growth rate is higher than at least 1 of the single agent growth rates=antagonistic. A p value <0.05 was considered statistically significant.

The interactions between Compound A and lenalidomide was additive. Meanwhile, female SCID mice bearing OCI-Ly10 xenografts could tolerate the treatments with Compound A and lenalidomide well whether they were given as single agents or in combination.

### Example 11

Anti tumor activity of Compound A as a single agent or in combination with ABT-199 in the Ly10 model.

Compound A, ABT-199, or vehicle were administered to female CB 17 SCID mice bearing OCI-LY 10 xenografts beginning on Day 0 for 21 days. Tumor growth inhibition was calculated on Day 21 of the study. The last measurement was taken on Day 46 of the study.

Compound A was administered once daily (QD), oral (PO), at 60 mg/kg for a total of 21 doses, which resulted in TGI=61.0% (ΔAUC, p<0.001). ABT-199 was administered once daily (QD), oral (PO), at 12.5 mg/kg for a total of 21 doses. It was found to have TGI=0% (ΔAUC, p<0.05). ABT-199 was administered once daily (QD), oral (PO), at 25 mg/kg for a total of 21 doses, which resulted in TGI=9.2% (ΔAUC, p<0.05). Compound A at 60 mg/kg in combination with ABT-199 at 12.5 mg/kg achieved TGI=83.7% (ΔAUC, p<0.001). The combination was found to be synergistic. Compound A at 60 mg/kg in combination with ABT-199 at 25 mg/kg achieved TGI=87.7% (ΔAUC, p<0.001). The combination was found to be synergistic.

Experimental design: Female CB17 SCID mice (Charles River Laboratories, about 21 g at treatment start) were inoculated subcutaneously in the flank (cell suspension) with 4.0×10⁶ OCI-Ly10 cells. Tumor growth was monitored with vernier calipers. Tumor volume was calculated using the formula V=W²×L/2, where V=volume, W=width and L=length of the tumor. When the mean tumor volume reached approximately 175 mm³, the animals were randomized into treatment groups (n=5/group). Mice were then dosed with 0.5% methylcellulose or Compound A or ABT-199 over a 21 day period (see Table 25). Tumor growth and body weight were measured twice per week. Tumor growth inhibition and body weight change were calculated on Day 21 of treatment.

Statistical analysis: The differences in the tumor growth trends over time between pairs of treatment groups were assessed using linear mixed effects regression models. These models account for the fact that each animal was measured at multiple time points. A separate model was fit for each comparison, and the areas under the curve (AUC) for each treatment group were calculated using the predicted values from the model. The percent decrease in AUC (dAUC) relative to the reference group was then calculated. A statistically significant P value suggests that the trends over time for the two treatment groups were different. Further details regarding pairwise comparisons are provided in the Example 2.

Drug combinations were assessed for synergy using observed AUC values. The change in AUC relative to the control was calculated for both single agent treatment groups as well as the combination group. The interaction between the two compounds was then assessed by comparing the change in AUC observed in the combination group to the sum of the changes observed in both single agents. The results can be divided into four categories: synergistic, additive, sub-additive, and antagonistic. Further details regarding combination analyses are provided in the Example 2.

Once a final analysis was selected, the tumor measurements observed on a date pre-specified by the researcher (typically the last day of treatment) were analyzed to assess tumor growth inhibition. For this analysis, a T/C ratio was calculated for each animal by dividing the tumor measurement for the given animal by the mean tumor measurement across all control animals. The T/C ratios across a treatment group were compared to the T/C ratios of the control group using a two-tailed Welch's t-test.

All P values <0.05 were called statistically significant in this Example.

Results and discussion: Compound A, ABT-199, or vehicle were administered to female CB 17 SCID mice bearing OCI-LY 10 xenografts beginning on Day 0 for 21 days. Tumor growth inhibition was calculated on Day 21 of the study. The last measurement was taken on Day 46 of the study.

Compound A was administered once daily (QD), oral (PO), at 60 mg/kg for a total of 21 doses, which resulted in TGI=61.0% (ΔAUC, p<0.001). ABT-199 was administered once daily (QD), oral (PO), at 12.5 mg/kg for a total of 21 doses. It was found to have TGI=0% (ΔAUC, p<0.05). ABT-199 was administered once daily (QD), oral (PO), at 25 mg/kg for a total of 21 doses, which resulted in TGI=9.2% (ΔAUC, p<0.05). Compound A at 60 mg/kg in combination with ABT-199 at 12.5 mg/kg achieved TGI=83.7% (ΔAUC, p<0.001). The combination was found to be synergistic. Compound A at 60 mg/kg in combination with ABT-199 at 25 mg/kg achieved TGI=87.7% (ΔAUC, p<0.001). The combination was found to be synergistic.

All of the groups were well tolerated. 1 animal was removed from Compound A (60 mg/kg) and ABT-199 (12.5 mg/kg) combination group (it was found dead). 1 animal was removed from ABT-199 (25 mg/kg) group (due to abnormal breathing and cold touch).

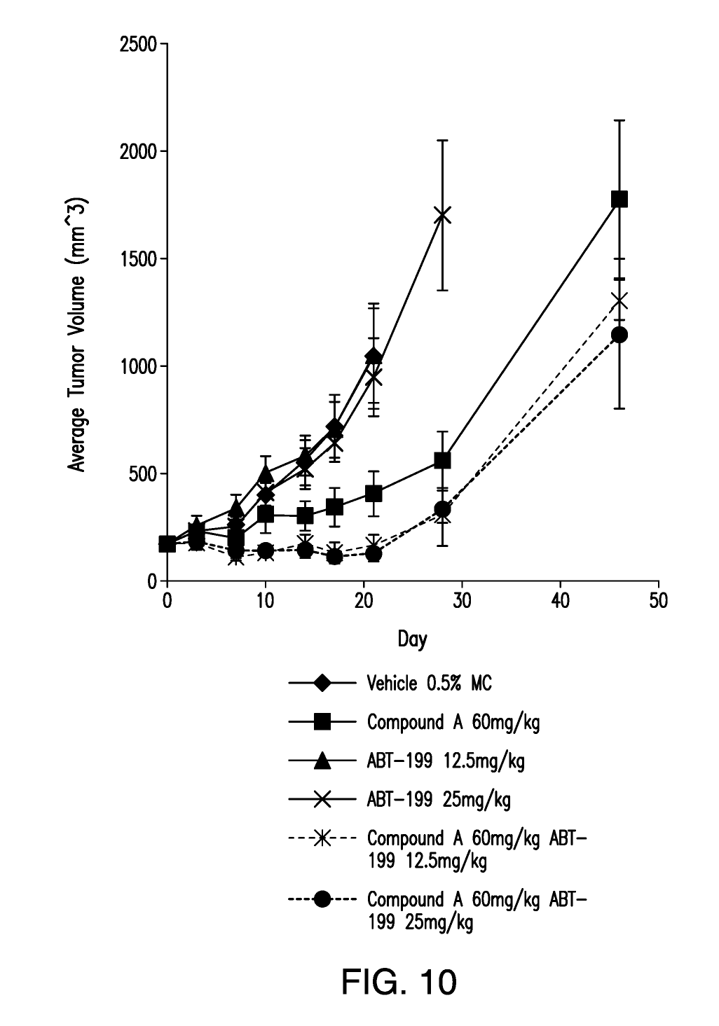

The antitumor activity of Compound A and ABT-199 alone or combined against Ly10 model is summarized in Table 25 and graphically presented in FIG. 10. The result of combination analysis is summarized in Table 26.

TABLE 25
Tumor growth inhibition.

| | | | | % BW^{a} | Number | Mean Tumor | | | |
|---|---|---|---|---|---|---|---|---|---|
| | | | Route | Change | of | Volume | | | P- |
| | Dose | | Schedule | (Day | animals | (mm³) ± | | TGI^{d} | Value^{e} |
| Test Article | (mg/kg) | Sequencing | Duration | Maximum) | removed | SEM^{b} | T/C^{c} | (%) | (dAUC) |
| 0.5% methyl- | N/A | | PO QD 21 | −1.1 (3) | 0 | 1,046.4 ± 243.4 | N/A | N/A | N/A |
| cellulose | | | | | | | | | |
| Compound A | 60.0 | | PO QD 21 | −0.0 (3) | 0 | 408 ± 104.9 | 0.39 | 61.0 | <0.001 |
| ABT-199 | 12.5 | | PO QD 21 | 4.6 (21) | 0 | 1,050.6 ± 221.4 | 1.00 | −0.4 | 0.025 |
| ABT-199 | 25.0 | | PO QD 21 | −1.5 (14) | 1 | 950.3 ± 182.1 | 0.91 | 9.2 | 0.046 |
| Compound A; | 60.0; | Compound | PO QD 21; | −3.7 (10) | 1 | 170.9 ± 43.6 | 0.16 | 83.7 | <0.001 |
| ABT-199 | 12.5 | A//ABT- | PO QD 21 | | | | | | |
| | | 199 | | | | | | | |
| Compound A; | 60; | Compound | PO QD 21; | 2.6 (17) | 0 | 128.9 ± 34.3 | 0.12 | 87.7 | <0.001 |
| ABT-199 | 25.0 | A//ABT- | PO QD 21 | | | | | | |
| | | 199 | | | | | | | |

TGI and T/C values were calculated on Day 21 of treatment.
^{a}% Body weight change (Day of Maximum change within the treatment period).
^{b}Standard error of the mean.
^{c}Treatment over control.
^{d}TGI = 100 − [(treated average volume/control average volume) × 100].
^{e}The changes in the areas under the tumor volume versus time curves (ΔAUCs) were assessed using linear mixed effects regression models to compare treatment groups with vehicle. P-values <0.05 indicate the percent decrease in AUC (dAUC) relative to the reference group was statistically significant.

TABLE 26
Combination comparisons (log-transformed).

| Comparison | Score | SEM | P-Value | Assess |
|---|---|---|---|---|
| Compound A (60 mg/kg) + | −87.8 | 28 | 0.013 | Syn. |
| ABT-199 (12.5 mg/kg) | | | | |
| Compound A (60 mg/kg) + | −75.8 | 24.4 | 0.01 | Syn. |
| ABT-199 (25.0 mg/kg) | | | | |

Statistically significant negative synergy scores indicate a synergistic combination (“Syn.”). Statistically significant positive synergy scores indicate a sub-additive combination (“Sub-add.”) when the combination performs better (i.e. has a lower AUC) than the best performing single agent. Statistically significant positive synergy scores indicate an antagonistic combination (“Antag.”) when the combination performs worse than the best performing single agent. Scores that are not statistically significant are considered additive (“Add.”). P values less than 0.05 were considered statistically significant.

### Example 12

Antitumor activity of Compound A and ibrutinib as single agents or in combination administered orally in female SCID mice bearing WSU-Luc human lymphoma xenografts.

Compound A, ibrutinib, or vehicle were administered to female SCID mice bearing WSU LUC xenografts beginning on Day 0 for 21 days either as single agents or in combination. The study ended on Day 20 of dosing. Compound A, ibrutinib, and vehicle were administered QD PO.

Compound A was administered QD PO at 60 mg/kg for a total of 21 doses and had TGI=37.6 (p<0.05). Ibrutinib was administered QD PO at 20 mg/kg for a total of 21 doses and had TGI=0.5 (p>0.05). Compound A was administered at 60 mg/kg in combination with ibrutinib at 20 mg/kg QD and this combination was considered additive, with TGI=20.7 (p>0.05).

Experimental design: Female CB17 SCID mice (Taconic Biosciences; weight at treatment start was about 18 g) were inoculated subcutaneously in the flank (cell suspension) with 4.0×10⁶ WSU-Luc cells. Tumor growth was monitored with vernier calipers. Tumor volume was calculated using the formula V=W²×L/2, where V=volume, W=width and L=length of the tumor. When the mean tumor volume reached approximately 190 mm³, the animals were randomized into treatment groups (n=8/group). Mice were then dosed with 0.5% methylcellulose or Compound A or ibrutinib over a 21 day period (see Table 27 for details). Tumor growth and body weight were measured twice per week. Tumor growth inhibition and body weight change were calculated on Day 20 of treatment.

Statistical analysis: The differences in the tumor growth trends over time between pairs of treatment groups were assessed using linear mixed effects regression models. These models account for the fact that each animal was measured at multiple time points. A separate model was fit for each comparison, and the areas under the curve (AUC) for each treatment group were calculated using the predicted values from the model. The percent decrease in AUC (dAUC) relative to the reference group was then calculated. A statistically significant P value suggests that the trends over time for the two treatment groups were different. Further details regarding pairwise comparisons are provided in the Example 2.

Drug combinations were assessed for synergy using observed AUC values. The change in AUC relative to the control was calculated for both single agent treatment groups as well as the combination group. The interaction between the two compounds was then assessed by comparing the change in AUC observed in the combination group to the sum of the changes observed in both single agents. The results can be divided into four categories: synergistic, additive, sub-additive, and antagonistic. Further details regarding combination analysis are provided in the Example 2.

Once a final analysis was selected, the tumor measurements observed on a date pre-specified by the researcher (typically the last day of treatment) were analyzed to assess tumor growth inhibition. For this analysis, a T/C ratio was calculated for each animal by dividing the tumor measurement for the given animal by the mean tumor measurement across all control animals. The T/C ratios across a treatment group were compared to the T/C ratios of the control group using a two-tailed Welch's t-test.

All P values <0.05 were called statistically significant in this Example. Days greater than 21 were excluded. All animals were included.

Results and discussion: Compound A as a single agent or in combination with ibrutinib, or vehicle were administered to female SCID mice bearing WSU-Luc xenografts beginning on Day 0 for 21 days. The study ended on Day 20 of dosing.

Compound A was administered QD PO at 60 mg/kg for a total of 21 doses and had TGI=37.6 (p<0.01). Ibrutinib was administered QD PO at 20 mg/kg for a total of 21 doses and had TGI=0.5 (p>0.05). Compound A was administered at 60 mg/kg in combination with ibrutinib at 20 mg/kg QD and this combination was considered additive, with TGI=20.7 (p>0.05).

All of the treatment groups were well tolerated with a maximal mean body weight loss of 4% in the vehicle treatment group, and less than 4% in all of the treatment groups. One animal was removed on Day 15 of treatment from the vehicle group due to body percentage weight loss.

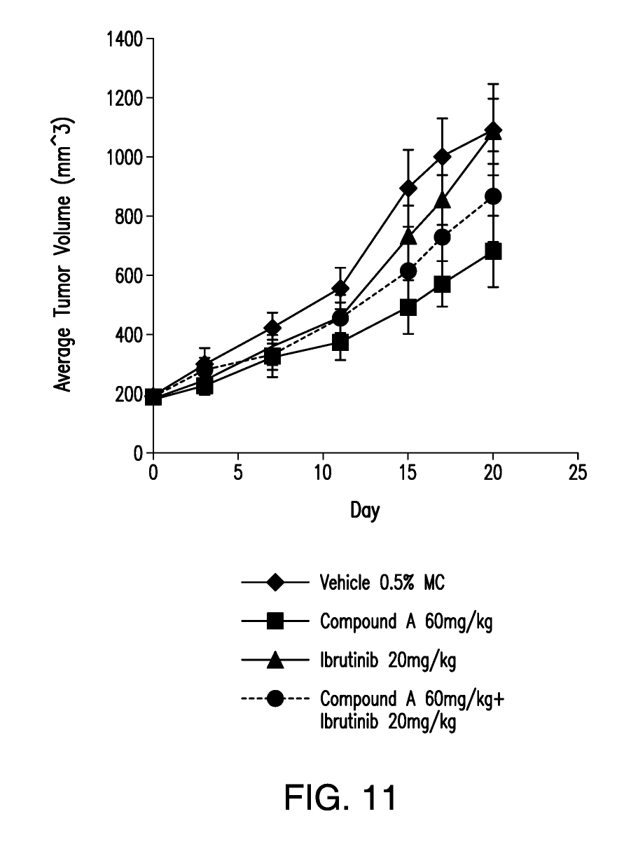

The antitumor activity of Compound A and ibrutinib alone or combined against WSU-Luc human lymphoma xenografts is summarized in Table 27 and graphically presented in FIG. 11. The result of combination analysis is summarized in Table 28.

TABLE 27
Tumor growth inhibition.

| | | | | % BW^{a} | Number | Mean Tumor | | | |
|---|---|---|---|---|---|---|---|---|---|
| | | | Route | Change | of | Volume | | | P- |
| | Dose | | Schedule | (Day | animals | (mm³) ± | | TGI^{d} | Value^{e} |
| Test Article | (mg/kg) | Sequencing | Duration | Maximum) | removed | SEM^{b} | T/C^{c} | (%) | (dAUC) |
| 0.5% methyl- | N/A | | PO QD 21 | −3.9 (11) | 1 | 1,090.2 ± 154.1 | N/A | N/A | N/A |
| cellulose | | | | | | | | | |
| Compound A | 60.0 | | PO QD 21 | 11.6 (20) | 0 | 680.4 ± 120.1 | 0.62 | 37.6 | 0.007 |
| ibrutinib | 20.0 | | PO QD 21 | 10.4 (20) | 0 | 1,085.2 ± 109.9 | 1.00 | 0.5 | 0.302 |
| Compound | 60.0; | Compound A// | PO QD 21; | −2.8 (11) | 0 | 864.7 ± 153.4 | 0.79 | 20.7 | 0.143 |
| A; ibrutinib | 20.0 | ibrutinib | PO QD 21 | | | | | | |

TGI and T/C values were calculated on Day 20 of treatment.
^{a}% Body weight change (Day of Maximum change within the treatment period).
^{b}Standard error of the mean.
^{c}Treatment over control.
^{d}TGI = 100 − [(treated average volume/control average volume) × 100].
^{e}The changes in the areas under the tumor volume versus time curves (ΔAUCs) were assessed using linear mixed effects regression models to compare treatment groups with vehicle. P-values <0.05 indicate the percent decrease in AUC (dAUC) relative to the reference group was statistically significant.

TABLE 28
Combination comparisons (log-transformed).

| Comparison | Score | SEM | P-Value | Assess |
|---|---|---|---|---|
| Compound A (60 mg/kg) + ibrutinib | 29.8 | 17 | 0.096 | Add. |
| (20 mg/kg) | | | | |

Statistically significant negative synergy scores indicate a synergistic combination (“Syn.”). Statistically significant positive synergy scores indicate a sub-additive combination (“Sub-add.”) when the combination performs better (i.e. has a lower AUC) than the best performing single agent. Statistically significant positive synergy scores indicate an antagonistic combination (“Antag.”) when the combination performs worse than the best performing single agent. Scores that are not statistically significant are considered additive (“Add.”). P values less than 0.05 were considered significant.

Combination of Compound A with ibrutinib was additive.

### Example 13

Antitumor activity of Compound A and rituximab alone or in combination in female SCID mice bearing Ly10 xenografts.

Summary: In the present study, the antitumor activity of Compound A given by oral gavage (PO) at 60 mg/kg once a day for 21 days (QD×21) was evaluated in female SCID mice bearing SC implanted Ly10 human lymphoma xenografts with or without rituximab (1 mg/kg) administered intravenously (IV) once a week for three weeks (QW×3). The antitumor activity of Compound A combined with rituximab was sub-additive. Animals survived the treatment with Compound A and rituximab alone or in combination.

Test articles: Compound A (purity >99% by weight; solid (white to off-white powder)) was stored at room temperature. Rituximab for injection (100 mg/10 mL; liquid) was stored at 4° C. The vehicle for Compound A was 0.5% methylcellulose. The vehicle for rituximab was 0.9% saline.

The dosing solutions preparations are summarized in Table 29.

TABLE 29
Compound A (for one week) and rituximab (for one dose)
dosing solution preparations.

| | Dose | Volume | | | Dosing |
|---|---|---|---|---|---|
| Compound | (mg/kg) | (mL/kg) | Conc. (mg/mL) | Route | Schedule |
| Compound A | 60 | 10 | 6.0 | PO | QD × 21 |
| rituximab | 1 | 10 | 0.1 | IV | Day 1, 8, 15 |

Vehicle for Compound A: 0.5% methylcellulose. The procedure for making Compound A at 6.0 mg/mL 80.0 mL solution was: (1) weigh 748.8 mg of Compound A powder; (2) add the powder in 80.0 mL of 0.5% methylcellulose; (3) sonicate the resulting off-white suspension for 5 minutes at room temperature and then vortex for 30 minutes; (4) check the pH and the value shall be pH=3.5; (5) store at room temperature and use it to dose animals for a week. Vortex the solutions well before each dosing.

The required volume for one dosing of rituximab was calculated as follows: 16 animals×20 g×10 mL/kg/1000×1.5=4.8 mL. The rituximab stock solution was 10 mg/mL (100 mg/10 mL). The procedure for making rituximab (0.1 mg/mL) 10 mL solution was: (1) take 0.1 mL of rituximab stock solution into a centrifuge tube; (2) add 9.9 mL of 0.9% saline and mix manually.

Dosing regimen: Table 30 shows the planned dosing regimens for each treatment group used in the study. Vehicle (0.5% methylcellulose) or Compound A was administered PO (QD×21). Rituximab was given IV (QW×3) on Day 1, 8, and 15. Dosing was initiated on Day 1 and continued up to Day 21 for animals completing the planned treatment regimen.

TABLE 30
Dosing regimen.

| | | Dose | Volume | Conc. | |
|---|---|---|---|---|---|
| N | Test Article | (mg/kg) | (mL/kg) | (mg/mL) | Route and Schedule |
| 8 | 0.5% | N/A | 10 | N/A | PO, QD × 21 days |
| | methylcellulose | | | | |
| 8 | Compound A | 60 | 10 | 6.0 | PO, QD × 21 days |
| 8 | rituximab | 1 | 10 | 0.1 | IV, QW × 3 |
| 8 | Compound A | 60 | 10 | 6.0 | PO, QD × 21 days |
| | rituximab | 1 | | 0.1 | IV, QW × 3 |

Data collection: Each animal (female SCID mice; Beijing HFK Bioscience Co., Ltd; group average weight at Day 0 was 19.5-20.2 g; acclimation period >5 days) was inoculated with 4×10⁶ Ly10 tumor cells (in 0.1 mL, 1:1 with Matrigel™) at the right flank for tumor model development. Body weight and tumor growth were monitored twice a week. Tumor size was measured to the nearest 0.1 mm using vernier calipers and applying the formula: V=W²×L/2, where V=volume, W=width and L=length of the tumor xenograft. Xenografts were allowed to grow until they reached an average size of approximately 250 mm³ after 20 days. Mice bearing the proper size xenograft were randomly assigned into one of the groups shown in Table 30 and began treatment with their assigned test material, either 0.5% methylcellulose, Compound A (60 mg/kg), rituximab (1 mg/kg), or the combination of Compound A plus rituximab for up to 21 days.

For this Example, passage was 17. The study was terminated on Day 39.

Results for body weight and tumor size measurement as well as the TGI calculation are summarized in Table 31 (body weight) and Table 32 (tumor growth inhibition), respectively.

Statistical tests: The differences in the tumor growth trends over time between pairs of treatment groups were assessed using linear mixed effects regression models. These models account for the fact that each animal was measured at multiple time points. A separate model was fit for each comparison, and the area under the curve (AUC) for each treatment group was calculated using the predicted values from the model. The percent decrease in AUC (dAUC) relative to the reference group was then calculated. A statistically significant P value suggests that the change over time for the two treatment groups was different.

Drug combinations were assessed for synergy using observed AUC values. The change in AUC relative to the control was calculated for both single agent treatment groups as well as the combination group. The interaction between the two compounds was then assessed by comparing the change in AUC observed in the combination group to the sum of the changes observed in both single agents. Statistically significant negative synergy scores indicate a synergistic combination (“Syn.”). Statistically significant positive synergy scores indicate a sub-additive or antagonistic combination (“Antag.”). Scores that are not statistically significant should be considered additive (“Add.”).

All P values <0.05 were called statistically significant in this Example. Further details regarding pairwise comparisons are provided in Example 2.

Results and discussion: In the present Example, no decrease in mean body weight was detected in female SCID mice bearing Ly10 xenografts from the vehicle-treated (0.5% methylcellulose, OD×21) control group during the 21-day treatment period. On Day 21, the mean body weight of the vehicle group increased 8.9% (or 1.7 g) compared to Day 0. The maximal decreases in mean body weight observed in single agent-treated groups were 7.1% (or 1.4 g, Day 21, for Compound A at 60 mg/kg, QD×21) and 1.5% (or 0.3 g, Day 21, for rituximab at 1 mg/kg, QW×3), respectively. Treatment with Compound A or rituximab alone resulted in TGI values of 76.0% (dAUC=122.4, P<0.001, for Compound A at 60 mg/kg) and 66.7% (dAUC=77.6, P<0.001, for rituximab at 1 mg/kg), respectively.

0.8% decrease (or 0.1 g, Day 3) in mean body weight was observed in the group treated with Compound A in combination with 1 mg/kg of rituximab. The combination treatment with 60 mg/kg of Compound A plus rituximab resulted in TGI value of 82.3% (dAUC=144.0, P<0.001, for Compound A plus rituximab at 1 mg/kg). Nevertheless, the antitumor activity of Compound A combined with rituximab was found to be only sub-additive in the present study.

Changes in animal body weight following the administration of Compound A and rituximab alone or in combination are summarized in Table 31. The antitumor activity of Compound A and rituximab alone or in combination against Ly10 xenografts is summarized in Table 32. The results of combination analysis are summarized in Table 33.

TABLE 31
Effects of Compound A and rituximab on animal body weight (g).

| Group | Day 0 | Day 3 | Day 7 | Day 10 | Day 14 | Day 17 | Day 21 |
|---|---|---|---|---|---|---|---|
| 0.5% | 19.5 | 20.0 | 19.9 | 20.3 | 20.6 | 20.7 | 21.2 |
| methylcellulose | Change | +2.9 | +2.5 | +4.4 | +5.7 | +6.2 | +8.9 |
| (QD × 21) | (%) | | | | | | |
| Compound A | 19.5 | 19.1 | 19.4 | 19.0 | 18.9 | 18.7 | 18.1 |
| (60 mg/kg, QD × | Change | −2.3 | −0.9 | −3.0 | −3.3 | −4.3 | −7.1 |
| 21) | (%) | | | | | | |
| rituximab | 19.8 | 19.5 | 19.9 | 19.8 | 19.9 | 19.8 | 19.5 |
| (1 mg/kg, QW × 3) | Change | −1.3 | +0.4 | −0.1 | +0.6 | +0.2 | −1.5 |
| | (%) | | | | | | |
| Compound A | 19.9 | 19.8 | 20.6 | 20.5 | 20.5 | 20.5 | 19.9 |
| (60 mg/kg, QD × | Change | −0.8 | +3.3 | +2.7 | +2.8 | +2.6 | +0.0 |
| 21) + | (%) | | | | | | |
| rituximab (1 mg/kg, | | | | | | | |
| QW × 3) | | | | | | | |

Data presented as Mean of 8 animals in each group

TABLE 32
Effects of Compound A and rituximab on tumor growth.

| | Mean Tumor | | | |
|---|---|---|---|---|
| | Volume | | | |
| | (mm³) ± SEM | TGI^{b} | | P Value |
| Group | at Day 21^{a} | (%) | dAUC | (dAUC)^{c} |
| 0.5% methylcellulose | 907.1 ± 44.4 | N/A | N/A | N/A |
| (QD × 21) | | | | |
| Compound A (60 mg/kg, | 217.6 ± 25.6 | 76.0 | 122.4 | <0.001 |
| QD × 21) | | | | |
| rituximab (1 mg/kg, | 301.8 ± 30.8 | 66.7 | 77.6 | <0.001 |
| QW × 3) | | | | |
| Compound A (60 mg/kg, | 160.6 ± 8.1 | 82.3 | 144.0 | <0.001 |
| QD × 21) + rituximab | | | | |
| (1 mg/kg, QW × 3) | | | | |

^{a}Data presented as Mean ± SEM of 8 animals in each group.
^{b}TGI = (V_{vehicle} − Vₜᵣₑₐₜₘₑₙₜ)/V_{vehicle} × 100% and values were calculated based on the measurements on Day 21.
^{c}The changes in the areas under the tumor volume versus time curves (ΔAUCs) were assessed using linear mixed effects regression models to compare treatment groups with vehicle. P-values < 0.05 indicate the percent decrease in AUC (dAUC) relative to the reference group was statistically significant.

TABLE 33
Results of combination analysis.

| | | | P- | |
|---|---|---|---|---|
| Comparison | Score | SEM | Value | Assess |
| Compound A (60 mg/kg, QD × 21) + | 55.3 | 10.4 | <0.001 | Sub- |
| rituximab (1 mg/kg, QW × 3) | | | | additive |

The antitumor activity of Compound A in combination with rituximab was sub-additive. Animals could tolerate the treatment with Compound A or rituximab alone or in combination.

### Example 14

Antitumor activity of Compound A and rituximab alone or in combination in female SCID mice bearing Ly19 xenografts.

In the present Example, the antitumor activity of Compound A administered by oral gavage (PO) at 60 mg/kg once a day for 14 days (QD×14) or rituximab at 10 mg/kg administered intravenously (IV) once a week for two weeks (QW×2) was evaluated in female SCID mice bearing SC implanted Ly19 human lymphoma xenografts. The results of the pairwise comparisons showed that, whether given alone or in combination, Compound A and rituximab treatment resulted in the tumor growth inhibition (TGI) values between 36.6% (for Compound A at 60 mg/kg alone) and 79.5% (for rituximab at 10 mg/kg alone). The antitumor activity of the combination treatment with Compound A and rituximab was antagonistic. Animals survived the treatment with Compound A and rituximab alone or in combination.

Test and control articles: Compound A (purity >99% by weight; solid (white to off-white powder)) was stored at room temperature. Rituximab for injection (100 mg/10 mL; liquid) was stored at 4° C. The vehicle for Compound A was 0.5% methylcellulose. The vehicle for rituximab was 0.9% saline.

The dosing solutions preparations are summarized in Table 34.

TABLE 34
Compound A (for one week) and rituximab (for one dose) dosing solution
preparations.

| | Dose | Volume | | | Dosing |
|---|---|---|---|---|---|
| Compound | (mg/kg) | (mL/kg) | Conc. (mg/mL) | Route | Schedule |
| Compound A | 60 | 10 | 6.0 | PO | QD × 14 |
| rituximab | 10 | 10 | 1 | IV | Day 1, 8 |

Vehicle for Compound A: 0.5% methylcellulose. The procedure for making Compound A at 6.0 mg/mL 60.0 mL solution was: (1) weigh 561.6 mg of Compound A powder; (2) add the powder in 60.0 mL of 0.5% methylcellulose; (3) sonicate the resulting off-white suspension for 5 minutes at room temperature and then vortex for 30 minutes; (4) check the pH and the value shall be pH=3.5; (5) store at room temperature and use it to dose animals for a week; (5) vortex the solutions well before each dosing.

The required volume for one dosing of rituximab was calculated as follows: 16 animals×20 g×10 mL/kg/1000×1.5=4.8 mL. The rituximab stock solution was 10 mg/mL (100 mg/10 mL). The procedure for making rituximab (0.1 mg/mL) 10 mL solution was: (1) take 0.5 mL of rituximab stock solution into a centrifuge tube; (2) add 4.5 mL of 0.9% saline and mix manually.

Dosing regimen: Table 35 shows the planned dosing regimens for each treatment group used in the study. Vehicle (5% methylcellulose) or Compound A was administered PO (QD×14). Rituximab was given IV (QW×2) on Day 1 and 8. Dosing was initiated on Day 1 and continued up to Day 14 for animals completing the planned treatment regimen.

TABLE 35
Dosing regimen.

| | | Dose | Volume | Conc. | |
|---|---|---|---|---|---|
| N | Test Article | (mg/kg) | (mL/kg) | (mg/mL) | Route and Schedule |
| 8 | 0.5% | N/A | 10 | N/A | PO, QD × 14 days |
| | methylcellulose | | | | |
| 8 | Compound A | 60 | 10 | 6.0 | PO, QD × 14 days |
| 8 | rituximab | 10 | 10 | 1.0 | IV, QW × 2 |
| 8 | Compound A | 60 | 10 | 6.0 | PO, QD × 14 days |
| | rituximab | 10 | 10 | 1.0 | IV, QW × 2 |

Data collection: Each animal (female SCID mice; Beijing HFK Bioscience Co., Ltd; group average weight at Day 0 was 18.9-19.9 g; acclimation period >5 days) was inoculated with 1×10⁶ Ly19 tumor cells (in 0.1 mL, 1:1 with Matrigel™) at the right flank for tumor model development. Body weight and tumor growth were monitored twice a week. Tumor size was measured to the nearest 0.1 mm using vernier calipers and applying the formula: V=W²×L/2, where V=volume, W=width and L=length of the tumor xenograft. Xenografts were allowed to grow until they reached an average size of approximately 137 mm³ after 6 days. Mice bearing the proper size xenograft were randomly assigned into one of the groups shown in Table 35 and began treatment with their assigned test material, either 5% methylcellulose, Compound A (60 mg/kg), rituximab (10 mg/kg), or the combination of Compound A plus rituximab for up to 14 days.

For this Example, passage was 17. The study was terminated on Day 14.

Results for body weight and tumor size measurement as well as the TGI calculation are summarized in Table 36 (body weight) and Table 37 (tumor growth inhibition), respectively.

Statistical tests: The differences in the tumor growth trends over time between pairs of treatment groups were assessed using linear mixed effects regression models. These models account for the fact that each animal was measured at multiple time points. A separate model was fit for each comparison, and the area under the curve (AUC) for each treatment group was calculated using the predicted values from the model. The percent decrease in AUC (dAUC) relative to the reference group was then calculated. A statistically significant P value suggests that the change over time for the two treatment groups was different.

Drug combinations were assessed for synergy using observed AUC values. The change in AUC relative to the control was calculated for both single agent treatment groups as well as the combination group. The interaction between the two compounds was then assessed by comparing the change in AUC observed in the combination group to the sum of the changes observed in both single agents. Statistically significant negative synergy scores indicate a synergistic combination (“Syn.”). Statistically significant positive synergy scores indicate a sub-additive or antagonistic combination (“Antag.”). Scores that are not statistically significant should be considered additive (“Add.”).

All P values <0.05 were called statistically significant in this Example. Further details regarding pair-wise comparisons are provided in Example 2.

Results and discussion: In the present Example, no decrease in mean body weight was detected in female SCID mice bearing Ly19 xenografts from the vehicle-treated (5% methylcellulose) control group on during the 14-day treatment period. On Day 14, the mean body weight of the vehicle group increased 16.6% (3.1 g) compared to Day 0. Similarly, no decrease in mean body weight was observed in animals treated with Compound A at 60 mg/kg (QD×14) alone or in combination with 10 mg/kg of rituximab (QW×2). The maximal decrease in mean body weight was 1.6% (0.3 g, Day 7) in animals treated with rituximab at 10 mg/kg alone (QW×2).

Treatment with Compound A or rituximab alone resulted in TGI values of 36.6% (dAUC=11.5, P<0.05, for Compound A at 60 mg/kg) and 79.5% (dAUC=61.8, P<0.001, for rituximab at 10 mg/kg). The combination treatment with Compound A and rituximab resulted in TGI value of 51.9% (dAUC=28.1, P<0.001, for Compound A at 60 mg/kg plus rituximab at 10 mg/kg). Nevertheless, the antitumor activity of Compound A combined with rituximab was found to be antagonistic in the present study (Score=47.1, P<0.01).

Changes in animal body weight following the administration of Compound A and rituximab alone or in combination are summarized in Table 36. The antitumor activity of Compound A and rituximab alone or in combination against Ly19 xenografts is summarized in Table 37. The results of combination analysis are summarized in Table 38.

TABLE 36
Effects of Compound A and rituximab on animal body weight (g).

| Group | Day 0 | Day 4 | Day 7 | Day 11 | Day 14 |
|---|---|---|---|---|---|
| 0.5% methylcellulose (QD × 14) | 18.9 | 19.5 | 19.7 | 20.6 | 22.0 |
| | Change (%) | +3.2 | +4.1 | +9.1 | +16.6 |
| Compound A (60 mg/kg, QD × 14) | 19.1 | 19.4 | 19.6 | 20.2 | 20.5 |
| | Change (%) | +2.0 | +3.1 | +6.2 | +7.4 |
| rituximab (10 mg/kg, QW × 2) | 19.4 | 19.3 | 19.1 | 19.7 | 19.9 |
| | Change (%) | −0.8 | −1.6 | +1.3 | +2.6 |
| Compound A (60 mg/kg, QD × 14) + | 19.5 | 19.7 | 19.7 | 19.9 | 20.3 |
| rituximab (10 mg/kg, QW × 2) | Change (%) | +0.7 | +1.0 | +1.9 | +3.7 |

Data presented as Mean of 8 animals in each group unless otherwise specified by the value in parenthesis.

TABLE 37
Effects of Compound A and rituximab on tumor growth in animals.

| | Mean Tumor | | | |
|---|---|---|---|---|
| | Volume | | | |
| | (mm³) ± SEM | TGI^{b} | | P Value |
| Group | at Day 14^{a} | (%) | dAUC | (dAUC)^{c} |
| 5% methylcellulose | 2516.1 ± 343.1 | N/A | N/A | N/A |
| (QD × 14) | | | | |
| Compound A (60 mg/kg, | 1594.0 ± 115.6 | 36.6 | 11.5 | <0.05 |
| QD × 14) | | | | |
| rituximab (10 mg/kg, | 516.8 ± 113.3 | 79.5 | 61.8 | <0.001 |
| QW × 2) | | | | |
| Compound A (60 mg/kg, | 1209.3 ± 145.8 | 51.9 | 28.1 | <0.001 |
| QD × 14) + rituximab | | | | |
| (10 mg/kg, QW × 2) | | | | |

^{a}Data presented as Mean ± SEM of 8 animals in each group unless otherwise specified by the value in parenthesis.
^{b}TGI = (V_{vehicle} − Vₜᵣₑₐₜₘₑₙₜ)/V_{vehicle} × 100% and values were calculated based on the measurements on Day 14.
^{c}The changes in the areas under the tumor volume versus time curves (ΔAUCs) were assessed using linear mixed effects regression models to compare treatment groups with vehicle. P-values < 0.05 indicate the percent decrease in AUC (dAUC) relative to the reference group was statistically significant.

TABLE 38
Results of combination analysis.

| | | | P- | |
|---|---|---|---|---|
| Comparison | Score | SEM | Value | Assess |
| Compound A (60 mg/kg, QD × 14) + | 47.1 | 15.4 | 0.008 | Antag. |
| rituximab (10 mg/kg, QW × 2) | | | | |

Due to fast tumor growth, the treatment lasted for only 14 days in the present study. The antitumor activity of Compound A in combination with rituximab was antagonistic. Animals could tolerate the treatment with Compound A and rituximab whether given alone or in combination.

### Example 15

Clinical non-Hodgkin lymphoma study design—a study of Compound A in combination with bendamustine, bendamustine and rituximab, gemcitabine, lenalidomide, or ibrutinib in subjects with advanced non-Hodgkin lymphoma.

Compound A is an orally bioavailable, potent and reversible inhibitor of SYK and Fms-like tyrosine kinase 3 (FLT3). SYK is a nonreceptor tyrosine kinase with SH2-binding domains that bind to phosphorylated ITAMs located on B and T cells and certain NK cells. SYK becomes activated upon ITAM binding and subsequently controls the activity of downstream signaling cascades that promote cell survival, growth, and proliferation, transcriptional activation, and cytokine release in these cell types. SYK is expressed ubiquitously in hematopoietic cells and abnormal function of SYK has been implicated in NHL, including follicular lymphoma (FL), DLBCL, and mantle cell lymphoma (MCL). Compound A inhibits SYK purified enzyme with an IC₅₀ of 3.2 nM and an EC₅₀ ranging from 25 to 400 nM in sensitive cell systems. Nonclinically, Compound A has exhibited antitumor activity in a number of mouse DLBCL xenograft models including the OCI-Ly10 model, an ABC-DLBCL model; the OCI-Ly19 model, a GCB-DLBCL model; the PHTX-95L model, a primary human DLBCL model; the RL FL model; and the MINO MCL model. Compound A has been tested in nonclinical DLBCL models in combination with a number of agents used in the relapsed/refractory setting, including gemcitabine, bendamustine, ibrutinib, and lenalidomide. With regard to clinical activity, in a recent study, 6 of the 20 response-evaluable subjects responded to treatment. Three subjects with DLBCL achieved a partial response (PR), 1 subject with FL achieved a complete response (CR), and 2 subjects with FL achieved stable disease (SD).

Gemcitabine HCl is a nucleoside analogue that primarily kills cells undergoing DNA synthesis (S-phase) and also blocks the progression of cells through the G1/S-phase boundary. Compound A has shown synergistic antitumor activity when combined with gemcitabine in nonclinical models. Ibrutinib is an inhibitor of BTK that is approved in CLL, MCL Waldenstrom's Macroglobulinemia and marginal zone lymphoma and is currently in clinical trials for DLBCL. It is hypothesized that targeting BTK, which lies downstream of SYK, in combination with SYK inhibition could lead to a more pronounced response in hematologic malignancies. In nonclinical animal models, the combination of Compound A with ibrutinib has shown synergistic antitumor activity. Bendamustine is a standard-of-care agent used in combination with rituximab as a second-line therapy to treat patients with NHL. Bendamustine has also shown synergistic TGI when combined with Compound A. Lenalidomide is an immunomodulatory agent that has been shown to modulate different components of the immune system by altering cytokine production, regulating T-cell co-stimulation, and augmenting the NK cell cytotoxicity. The immunomodulatory properties of lenalidomide are implicated in its clinical efficacy and provide a rationale for combination with Compound A. Nonclinical combination of these agents has shown additive tumor inhibition in mouse models. Overall, data from nonclinical sources support the potential for Compound A to be an effective agent in treating patients with relapsed or refractory NHL in combination with gemcitabine, bendamustine, ibrutinib, or lenalidomide.

Study design: This is a phase 1b, dose escalation study of Compound A in combination with bendamustine, bendamustine+rituximab, gemcitabine, lenalidomide, or ibrutinib (Cohorts A-E) in adult patients with advanced non-Hodgkin lymphoma (NHL) after at least 1 prior line of therapy. The primary objective of the study is to determine the maximum tolerated dose (MTD) or the recommended phase 2 dose (RP2D) of Compound A when administered in each of the combinations.

During dose escalation, the dose of Compound A will be escalated (2 planned dose levels of escalation: 60 and 100 mg) according to a 3+3 dose escalation scheme; bendamustine, bendamustine+rituximab, gemcitabine, lenalidomide, and ibrutinib will be administered at a fixed dose and regimen. Dosing will increase to 100 mg once daily (QD), provided that the safety and tolerability of the 60 mg dose has been demonstrated. Intermediate dose levels between 60 and 100 mg in increments of 20 mg (e.g., 80 mg) or dose levels below the starting dose of 60 mg (e.g., 40 mg) also may be evaluated if appropriate. Dose escalation will continue until the MTD is reached, or until Compound A 100 mg QD (the maximally administered dose [MAD]) is determined to be safe and tolerable, or until an RP2D, if different from the MTD or MAD, is identified on the basis of the safety, tolerability, and preliminary pharmacokinetic (PK) and efficacy data (if available) observed in Cycle 1 and beyond. Approximately 6 additional patients will be added at the MTD/MAD/RP2D for each combination for further safety evaluation. Serial PK samples will be collected at prespecified time points in Cycle 1 to characterize the PK of Compound A when given with each of the combination regimens. Toxicity will be evaluated according to National Cancer Institute Common Terminology Criteria for Adverse Events version 4.03. Common Terminology Criteria for Adverse Events (CTCAE). National Cancer Institute, National Institutes of Health, U.S. Department of Health and Human Services Series v4.03. Jun. 14, 2010. Publication No. 09-5410.

Primary objective: To determine the MTD or RP2D for Compound A when administered in combination with bendamustine, bendamustine+rituximab, gemcitabine, lenalidomide, or ibrutinib. Secondary objectives: To characterize the plasma PK of Compound A when administered in each of the combinations and to observe the preliminary efficacy of Compound A in patients with advanced lymphoma who have relapsed and/or are refractory after ≥1 prior line of therapy. Additional objective: To evaluate safety and tolerability of Compound A in combination with bendamustine, bendamustine+rituximab, gemcitabine, lenalidomide, or ibrutinib. Exploratory objectives: To evaluate response-predictive biomarkers including, but not limited to, cell of origin classification, somatic mutations, copy number changes, and gene expression. To assess germline polymorphic variations in genes encoding drug-metabolizing enzymes and/or transporters involved in the metabolism or disposition of Compound A. To assess the pharmacodynamic effects of Compound A by measuring basal and postdose levels of circulating cytokines/chemokines in subjects with lymphoma malignancies.

Subject population: Patients with advanced NHL of any histology after at least 1 prior line of therapy. Number of subjects: Approximately 100 patients (˜20 patients per arm). Number of sites: estimated total of 15 sites in North America and Europe.

Dose level(s): Compound A: planned 60 or 100 mg orally (PO) QD plus one of the following: bendamustine: 90 mg/m² administered intravenously (IV) over 10 or 60 minutes (depending on which formulation is used) on Days 1 and 2 of a 21-day cycle, up to 8 cycles; bendamustine+rituximab: 90 mg/m² bendamustine administered IV 10 or 60 minutes (depending on which formulation is used) on Days 1 and 2 of a 21-day cycle, up to 8 cycles, and 375 mg/m² rituximab administered IV per local guidelines and labeling on Day 1 of a 21-day cycle, up to 8 cycles; gemcitabine: 1000 mg/m² IV infusion over 30 minutes on Days 1 and 8 of a 21-day cycle; lenalidomide: 25 mg PO QD for Days 1 to 21 of a 28-day cycle; or ibrutinib: 560 mg PO QD of a 28-day cycle.

Route of administration: Compound A PO, bendamustine IV, rituximab IV, lenalidomide PO, gemcitabine IV, and ibrutinib PO. Duration of treatment: Treatment will continue until disease progression, unacceptable toxicities, or withdrawal due to other reasons. Estimated treatment duration is 12 months. Period of evaluation: Patients will be followed for safety for 28 days after the last dose of study drug or until the start of subsequent anticancer therapy, whichever occurs first.

Study population: Subjects must have a histologically or cytologically confirmed diagnosis of advanced NHL of any histology (with the exception of patients with Waldenstrom macroglobulinemia (WM) and chronic lymphocytic leukemia (CLL)), radiographically or clinically measurable disease with at least 1 target lesion per International Working Group (IWG) criteria for malignant lymphoma, and must be refractory or relapsed after at least 1 prior line of therapy and have no effective standard therapy available per investigator's assessment. CHESON et al., J. Clin. Oncol., 25(5):579-586 (2007). Subjects must also be either treatment naïve to, relapsed/refractory to, or have experienced treatment failure due to other reasons with ibrutinib, idelalisib, or any other investigational B-cell receptor (BCR) pathway inhibitors not directly targeting SYK.

Inclusion criteria: Each patient must meet all the following inclusion criteria to be enrolled in the study. (1) Male or female patients aged 18 years or older. (2) Histologically or cytologically confirmed diagnosis of advanced NHL of any histology (with the exception of patients with WM and CLL). (3) Radiographically or clinically measurable disease with at least 1 target lesion per IWG criteria for malignant lymphoma. (4) Patients who are refractory or relapsed after at least 1 prior line of therapy and for whom no effective standard therapy is available per the investigator's assessment: (a) either treatment naïve to, relapsed/refractory to, or experienced treatment failure due to other reasons with ibrutinib, idelalisib, or any other investigational BCR pathway inhibitors not directly targeting SYK; (b) prior treatment with a regimen that includes the combination drug will not necessarily exclude a patient from that cohort if the investigator views treatment with that agent as appropriate; however, a patient who has a contraindication for a particular combination agent or who has been discontinued from prior therapy with a particular agent for toxicity will not be eligible for inclusion in that particular cohort. (5) Eastern Cooperative Oncology Group (ECOG) performance status score of 0 or 1 and life expectancy of greater than 3 months. OKEN et al. Am. J. Clin. Oncol., 5(6):649-655 (1982). (6) Patients must have adequate organ function, including the following: (a) Adequate bone marrow reserve: absolute neutrophil count (ANC)≥1000/μL, platelet count ≥75,000/μL (≥50,000/μL for patients with bone marrow involvement), and hemoglobin ≥8 g/dL (red blood cell [RBC] and platelet transfusion allowed ≥14 days before assessment). (b) Hepatic: total bilirubin ≤1.5×the upper limit of the normal range (ULN); alanine aminotransferase (ALT) and AST≤2.5×ULN. (c) Renal: creatinine clearance ≥60 mL/min either as estimated by the Cockcroft-Gault equation or based on urine collection (12 or 24 hours). (d) Lipase ≤1.5×ULN and amylase ≤1.5×ULN with no clinical symptoms suggestive of pancreatitis or cholecystitis. (e) Blood pressure ≤Grade 1 (hypertensive subjects are permitted if their blood pressure is controlled to ≤Grade 1 by hypertensive medications and glycosylated hemoglobin [HbA1C]≤6.5%). COCKCROFT et al., Nephron, 16(1):31-41 (1976).

(7) Female patients who: (a) are postmenopausal for at least 1 year before the screening visit, or (b) are surgically sterile, or (c) if they are of childbearing potential, agree to practice 1 highly effective method of contraception and 1 additional effective (barrier) method at the same time, from the time of signing the informed consent through 180 days after the last dose of study drug, or (d) agree to practice true abstinence, when this is in line with the preferred and usual lifestyle of the subject. European Heads of Medicines Agencies (HMA) Clinical Trial Facilitation Group (CTFG), Recommendations related to contraception and pregnancy testing in clinical trials (2014), available at hma.eu/fileadmin/dateien/Human_Medicines/01-About_HMA/Working_Groups/CTFG/2014_09_HMA_CTFG_Contraception.pdf. Periodic abstinence (e.g., calendar, ovulation, symptothermal, postovulation methods), withdrawal, spermicides only, and lactational amenorrhea are not acceptable methods of contraception. Female and male condoms should not be used together. Male patients, even if surgically sterilized (i.e., status postvasectomy), who: (a) agree to practice effective barrier contraception during the entire study treatment period and through 180 days after the last dose of study drug, or (b) agree to practice true abstinence, when this is in line with the preferred and usual lifestyle of the subject. Periodic abstinence (e.g., calendar, ovulation, symptothermal, postovulation method), withdrawal, spermicides only, and lactational amenorrhea are not acceptable methods of contraception. Female and male condoms should not be used together. (8) Both men and women in the rituximab combination arm (Cohort B) must practice contraception as described above from the time of signing of the informed consent form (ICF) through 12 months after the last dose of study drug. (9) Both men and women in the lenalidomide combination arm (Cohort D) must adhere to the guidelines of the RevAssist program or, if not using commercial supplies, must adhere to a similar program. (10) Voluntary written consent must be given before performance of any study-related procedure not part of standard medical care, with the understanding that consent may be withdrawn by the patient at any time without prejudice to future medical care. (11) Recovered (i.e., <Grade 1 toxicity) from the reversible effects of prior anticancer therapy.

Exclusion criteria: Subjects meeting any of the following exclusion criteria are not to be enrolled in the study. (1) Central nervous system (CNS) lymphoma; active brain or leptomeningeal metastases, as indicated by positive cytology from lumbar puncture or CT scan/magnetic resonance imaging (MRI). Exceptions include those subjects who have completed definitive therapy, are not on steroids, have a stable neurologic status for at least 2 weeks after completion of the definitive therapy and steroids and do not have neurologic dysfunction that would confound the evaluation of neurologic and other adverse events (AEs). (2) Known human immunodeficiency virus (HIV)-related malignancy. (3) Known hypersensitivity (e.g., anaphylactic and anaphylactoid reactions) to any particular combination drug will result in a patient being ineligible for inclusion in that particular cohort. (4) For patients in the lenalidomide combination arm, demonstrated hypersensitivity (e.g., angioedema, Stevens-Johnson syndrome, toxic epidermal necrolysis) to lenalidomide. (5) History of drug-induced pneumonitis requiring treatment with steroids; history of idiopathic pulmonary fibrosis, organizing pneumonia, or evidence of active pneumonitis on screening chest CT scan; history of radiation pneumonitis in the radiation field (fibrosis) is permitted. (6) Life-threatening illness unrelated to cancer that could, in the investigator's opinion, make the patient not appropriate for this study. (7) Female patients who are lactating and breast-feeding or a positive serum pregnancy test during the screening period or a positive urine pregnancy test on Day 1 before the first dose of study drug. (8) Any serious medical or psychiatric illness, including drug or alcohol abuse, that could, in the investigator's opinion, potentially interfere with the completion of treatment according to this protocol. (9) Known human immunodeficiency virus (HIV) positive. (10) Known hepatitis B surface antigen positive, or known or suspected active hepatitis C infection. (11) Systemic anticancer treatment (including investigational agents) or radiotherapy less than 2 weeks before the first dose of study treatment (≤4 weeks for large molecule agents) or not recovered from acute toxic effects from prior chemotherapy and radiotherapy. (12) Prior treatment with investigational agents ≤21 days or ≤5 times their half-life (whichever is shorter) before the first dose of study drug. (13) Prior autologous stem cell transplant (ASCT) within 6 months or prior ASCT at any time without full hematopoietic recovery before Cycle 1 Day 1, or allogeneic stem cell transplant any time.

(14) Any clinically significant comorbidities, such as uncontrolled pulmonary disease, known impaired cardiac function or clinically significant cardiac disease (specified below), active CNS disease, active infection, or any other condition that could compromise the patient's participation in the study. Patients with any of the following cardiovascular conditions are excluded: (a) acute myocardial infarction within 6 months before starting study drug; (b) current or history of New York Heart Association Class III or IV heart failure; (c) evidence of current, uncontrolled cardiovascular conditions including cardiac arrhythmias, angina, pulmonary hypertension, or electrocardiographic evidence of acute ischemia or active conduction system abnormalities; (d) Friderichia corrected QT interval (QTcF)>450 milliseconds (msec) (men) or >475 msec (women) on a 12-lead ECG during the Screening period; (e) abnormalities on 12-lead ECG including, but not limited to, changes in rhythm and intervals that, in the opinion of the investigator, are considered to be clinically significant. The Criteria Committee of New York Heart Association. Nomenclature and Criteria for Diagnosis of Diseases of the Heart and Great Vessels. Ninth Ed. Boston, Mass.: Little, Brown & Co; 1994:253-256.

(15) For patients in all combination arms (Cohorts A-E), use or consumption of any of the following substances: (a) Medications or supplements that are known to be inhibitors of P-gp and/or strong reversible inhibitors of CYP3A within 5 times the inhibitor half-life (if a reasonable half-life estimate is known) or within 7 days (if a reasonable half-life estimate is unknown) before the first dose of study drug. In general the use of these agents is not permitted during the study except in cases in which an AE must be managed. See, e.g., U.S. Food and Drug Administration, Drug Interaction Studies—Study Design, Data Analysis, Implications for Dosing, and Labeling Recommendations, Draft Guidance (2012), available at http://www.fda.gov/downloads/Drugs/GuidanceComplianceRegulatoryInformation/Guidances/U CM292362.pdf; U.S. Food and Drug Administration, Drug Development and Drug Interactions, available at http://www.fda.gov/Drugs/DevelopmentApprovalProces/DevelopmentResources/%20DrugInter actionsLabeling/ucm080499.htm. (b) Medications or supplements that are known to be strong CYP3A mechanism-based inhibitors or strong CYP3A inducers and/or P-gp inducers within 7 days or within 5 times the inhibitor or inducer half-life (whichever is longer) before the first dose of study drug. In general the use of these agents is not permitted during the study except in cases in which an AE must be managed. (c) Grapefruit-containing food or beverages within 5 days before the first dose of study drug. Note that grapefruit-containing food and beverages are not permitted during the study.

(16) Additionally, for patients in the ibrutinib combination arm (Cohort E), use or consumption of any of the following substances: (a) Medications or supplements that are known to be moderate reversible inhibitors of CYP3A within 5 times the inhibitor half-life (if a reasonable half-life estimate is known) or within 7 days (if a reasonable half-life estimate is unknown) before the first dose of study drugs. In general the use of these agents is not permitted during the study for this combination except in cases in which an AE must be managed. (b) Medications or supplements that are known to be moderate mechanism-based inhibitors or moderate inducers of CYP3A within 7 days or within 5 times the inhibitor or inducer half-life (whichever is longer) before the first dose of study drugs. In general the use of these agents is not permitted during the study for this combination except in cases in which an AE must be managed. (c) Seville oranges within 5 days before the first dose of study drugs and during the study.

(17) Major surgery within 14 days before the first dose of study drug and not recovered fully from any complications from surgery. (18) Systemic infection requiring IV antibiotic therapy or other serious infection within 14 days before the first dose of study drug. (19) Patients with another malignancy within 2 years of study start. Patients with nonmelanoma skin cancer or carcinoma in situ of any type are not excluded if they have undergone complete resection and are considered disease-free at the time of study entry. (20) Known GI disease or GI procedure that could interfere with the oral absorption or tolerance of Compound A including difficulty swallowing tablets or diarrhea >Grade 1 despite supportive therapy. (20) Treatment with high-dose corticosteroids for anticancer purposes within 14 days before the first dose of Compound A; daily dose equivalent to 10 mg oral prednisone or less is permitted. Corticosteroids for topical use or in nasal spray or inhalers are allowed.

Main criteria for evaluation and analyses: Primary: (1) MTD (dose escalation); (2) RP2D (dose escalation). Secondary: (1) summary statistics of Compound A maximum observed concentration (Cmax) on Cycle 1 Days 1 and 15 by dose escalation cohort; (2) summary statistics of Compound A time of first occurrence of Cmax (Tmax) on Cycle 1 Days 1 and 15 by dose escalation cohort; (3) summary statistics of Compound A area under the plasma concentration-time curve during a dosing interval (AUCτ) on Cycle 1 Days 1 and 15 by dose escalation cohort; (4) objective response rate; (5) duration of response; (6) time to progression. Additional: (1) percentage of patients with AEs. (2) percentage of patients with ≥Grade 3 AEs. (3) percentage of patients with serious adverse events. (4) percentage of patients who discontinue due to AEs. (5) clinically significant laboratory values. (6) clinically significant vital sign measurement. (6) summary statistics of Compound A apparent oral clearance, peak-trough ratio, accumulation ratio, and trough concentration on Cycle 1 Day 15 by dose escalation cohort. (7) summary statistics of Compound A plasma concentrations on Cycle 1 Days 1 and 15 by dose escalation cohort. Exploratory: (1) candidate response-predictive biomarker(s) for any of the combinations tested in the study including, but not limited to, cell of origin classification, specific somatic mutation(s), signature(s), defined by copy number changes and/or gene expression, and other disease-relevant molecular biomarkers with diagnostic and prognostic value, such as BCL-2, MYC, BCL-6, and Ki-67. (2) Pharmacodynamic biomarkers include a panel of cytokines/chemokines in patient blood, which include, but not limited to, B-cell receptor-mediated cytokines/chemokines. (3) Germline polymorphisms in genes encoding drug-metabolizing enzymes and/or transporters involved in the metabolism or disposition of Compound A.

Statistical considerations: For each combination, dose escalation will be conducted according to a standard 3+3 dose escalation schema, and approximately 12 dose-limiting toxicity (DLT)-evaluable patients will be enrolled. Dose escalation will continue until the MTD is reached or until Compound A 100 mg QD (the MAD) is determined to be safe and tolerable, or until an RP2D, if different from the MTD or MAD, is identified on the basis of the safety, tolerability, and preliminary PK and efficacy data (if available) observed in Cycle 1 and beyond. The MTD/MAD/RP2D cohort will have at least 6 patients. The escalation of each of the combination cohorts is independent of the other cohorts.

Sample size justification: The dose escalation of this study will use a 3+3 design. Rituximab, gemcitabine, lenalidomide, and ibrutinib dose will be the dose according to the manufacturer's label. The dose of bendamustine will be based on its use in previous combination studies. RUMMEL et al., J. Clin. Oncol., 23(15):3383-3389 (2005); VACIRCA et al., Ann. Hematol., 93(3):403-409 (2014). The planned doses of Compound A are 60 and 100 mg. For each combination arm, 9 to 12 DLT-evaluable patients will be needed for the dose escalation portion. In addition, for each arm, another 6 patients will be needed for safety expansion. Assuming a 10% dropout rate, 20 patients will be needed for each arm; therefore, the total sample size for this study, including all 5 arms, will be 100.

### Example 16

Clinical study design—a study of Compound A in combination with nivolumab in subjects with advanced solid tumors.

Recent studies have shown that myeloid-derived suppressor cells (MDSCs) use CD79-ITAM signaling that uses SYK as a signaling mediator. Additionally, Fms-like tyrosine kinase 3 (FLT3) and its ligand have been shown to induce MDSCs in vitro. LECHNER et al., J. Transl. Med. 9:90 (2011).

MDSC-mediated immune suppression has been reported in many solid tumors, including, but not limited to breast cancer, head and neck, and NSCLC. COTECHINI et al., Cancer. J., 21(4): 343-50 (2015). In addition, a role of B cells in tumor immunity has been studied, and the requirement of B cells for tumor growth and metastasis has been documented. DILILLO et al., J. Immunol., 184(7):4006-4016 (2010). SYK is known to be essential for development, growth, and maintenance of B cells.

SYK inhibition results in the loss of MDSCs and activation of T-cell response, both in vitro and in vivo. See, e.g., LUGER et al., PLoS One, 8(10):e76115 (2013). Although preclinical experiments in tumors in which SYK-mediated MDSC or B-cell immunosuppression are active show that Compound A did not inhibit or activate T cells directly, in certain embodiments, synergistic activity may be observed when a PD-1 receptor inhibitor that promotes T-cell function is administered in combination with a SYK inhibitor. The benefit of administering an anti-PD-1 agent with Compound A has been observed nonclinically and this effect may be indirectly attributed to the decrease in CD11b+MDSC or B220+B cells in the tumor-infiltrating immune cells of Compound A-treated tumors. In particular, this combination was evaluated in in vivo CT26 mouse syngeneic colon cancer model. In this prior non-clinical study, 80% of the animals treated with Compound A (60 mg/kg) in combination with anti-PD-1 (dosed at 5 mL/kg, intraperitoneal administration (IP) for final dose of 10 mg/kg) were tumor free, even 30 days after the last dose was administered. This shows that the combination resulted in a complete cure for a prolonged period of time. In comparison, only 20% of animals treated with Compound A alone survived to 30 days after the last dose, and only 30% of animals treated with anti PD-1 antibody alone survived to 30 days after the last dose.

Accordingly, the addition of a SYK inhibitor, such as Compound A, to an anti-PD-1 agent may improve tumor regression via modulation of the tumor-infiltrating immune cells and other immune cells in the tumor microenvironment. The make-up of the tumor microenvironment, which differs among different tumor types, informs the choice of diseases to be evaluated in this study. Tumors in which there is MDSC or B-cell suppression are of particular interest: nonclinical assessment suggests that tumors such as triple-negative breast cancer (TNBC), non-small cell lung cancer (NSCLC), and head and neck squamous cell carcinoma (HNSCC) show MDSC-mediated tumor immunosuppression. Because the tumor microenvironment informs the choice of diseases to be evaluated, the results of this clinical study can extend to other cancers.

The results of a clinical study of Compound A in combination with nivolumab in subjects with advanced solid tumors such as such as TNBC, NSCLC, and HNSCC can inform about various parameters (e.g., MTD and RP2D) for administration of Compound A in combination with nivolumab in patients with other cancers. For example, such clinical study can be used to determine parameters (e.g., MTD and RP2D) for administration of Compound A in combination with nivolumab in patients with DLBCL. Such clinical study can also be used to determine parameters for administration of Compound A in combination with nivolumab in patients with an NHL, with an NHL other than CLL, CLL, iNHL, MCL, or PTLD.

The study intends to clinically evaluate the combination effect of Compound A and nivolumab in 3 advanced solid tumor types (TNBC, NSCLC, and HNSCC). With the reference response rate of ˜20% observed with nivolumab monotherapy in each of these indications, added benefit of this combination regimen will be assessed by whether a significantly improved overall response rate (ORR; target ORR of 40%) is observed, along with assessment of other efficacy measures such as duration of response (DOR) and survival benefit. While the majority of the subjects in each of the cohorts (24/30 response-evaluable subjects) should be naïve to anti-PD-1, anti-PD-L1, and any other immune-directed antitumor therapies, a small subset of 6/30 response-evaluable subjects with prior exposure to an anti-PD-1 or anti-PD-L1 agent will be enrolled to observe any combination effect of Compound A plus nivolumab in this relapsed/refractory setting with regard to the PD-1/PD-L1 blockade. In addition, after determining a safe and tolerable combination dose for Compound A when co-administered with nivolumab during the initial dose escalation stage of the study, a 2-week, single-agent Compound A treatment period before combination therapy is planned in 10 response-evaluable subjects in each of the expansion cohorts. Correlative science studies are planned in pretreatment and post-treatment tumor biopsies and peripheral blood samples taken from these subjects with the intent of having more mechanistic understanding of the role of SYK in the tumor immunity and direct tumor effect, if any.

This is an open-label, multicenter, phase 1b, dose escalation study of Compound A in combination with nivolumab in subjects with advanced solid tumors. The purpose of this study is to evaluate the maximum tolerated dose (MTD) or recommended Part 2 dose (RP2D), safety and efficacy of Compound A in combination with nivolumab in subjects with advanced solid tumors. The results of this study can be used to determine parameters (e.g. MTD and RP2D) for administration of Compound A in combination with nivolumab in subjects with DLBCL. The results can also be used to determine parameters for administration of Compound A in combination with nivolumab in patients with an NHL, with an NHL other than CLL, CLL, iNHL, MCL, or PTLD.

The drug being tested is Compound A. This study will look at the determination of MTD/RP2D and efficacy measured by overall response rate (ORR) in subjects who take Compound A in combination with nivolumab. The study will include a dose escalation phase (Part 1) and a dose expansion phase (Part 2).

All subjects will be asked to take the tablets of Compound A at the same time each day throughout the study. Subjects will also receive intravenous infusion of nivolumab at the same time once every 2 weeks. This multi-center trial will be conducted globally. The overall time to receive treatment in this study is approximately 12 months. Subjects will be assessed for disease response and PD during the progression-free survival (PFS) follow-up of 6 months (for subjects who discontinue due to reasons other than PD) and OS follow-up of 12 months from the last dose of study drug.

The study will include a dose escalation phase (Part 1) and a dose expansion phase (Part 2). In the dose escalation phase, the subject population will consist of all-comer subjects with advanced solid tumors for whom 1 or more prior lines of therapy have failed and who have no effective therapeutic options available based on investigator assessment. The dose expansion phase will include 3 cohorts: (1) subjects with metastatic triple-negative breast cancer (TNBC) who have had ≥1 prior line of chemotherapy; (2) subjects with locally advanced or metastatic non-small cell lung cancer (NSCLC) that has progressed on or after a prior platinum-based chemotherapy; and (3) subjects with locally advanced or metastatic head and neck squamous cell carcinoma (HNSCC) that has progressed or recurred within 6 months of the last platinum-based chemotherapy.

It is expected that approximately 120 subjects will be enrolled in the study: approximately 9 to 12 subjects in the dose escalation cohort and approximately 36 subjects (30 evaluable subjects+15% drop off) in each of the 3 dose expansion cohorts. Subjects will be assigned to 1 of the 4 treatment groups: Part 1 Compound A+Nivolumab; Part 2 Metastatic TNBC; Part 2 Metastatic NSCLC; and Part 2 Metastatic HNSCC.

Once enrolled in the study, subjects will be administered Compound A orally once daily during each 28-day treatment cycle. Subjects receiving the combination therapy will also receive nivolumab once every 2 weeks intravenously (IV) over 60 minutes on Day 1 and Day 15 of each 28-day treatment cycle (for subjects who receive 2 weeks of Compound A monotherapy before starting combination treatment, the first nivolumab infusion will be administered on Cycle 1 Day 15). On days when both Compound A and nivolumab will be administered, the Compound A dose will be administered first followed by the nivolumab infusion (infusion to begin within 30 minutes after the Compound A dose). Subjects, including those who achieve a complete response, may receive study treatment until they experience disease progression (PD) or unacceptable toxicities.

The dose of nivolumab will be 3 mg/kg IV. The starting dose of Compound A will be 60 mg QD. Dose escalation will follow a standard 3+3 escalation scheme, and dosing will increase to 100 mg QD, provided that the safety and tolerability of the 60 mg dose has been demonstrated. Intermediate dose levels between 60 and 100 mg (e.g., 80 mg) or dose levels below the starting dose of 60 mg (e.g., 40 mg) may be also evaluated if appropriate. Dose escalation will continue until the maximum tolerated dose (MTD) is reached, or until 100 mg QD of Compound A (the maximally administered dose, (MAD)) is determined to be safe and tolerable, or until a recommended phase 2 dose (RP2D), if different from the MTD or MAD, has been identified on the basis of the safety, tolerability, and preliminary pharmacokinetic (PK) and efficacy data (if available) observed in Cycle 1 and beyond. At least 6 subjects will be evaluated at the RP2D (the MTD, MAD, or a lower dose as determined) before making a decision to advance to further dose expansion.

After the combination RP2D (the MTD, MAD, or a lower dose) is determined, expansion cohorts are planned in subjects with TNBC, NSCLC, and HNSCC. Thirty response-evaluable subjects will be enrolled in each expansion cohort, including approximately 10 subjects in each cohort who are able to provide evaluable serial biopsies. Additionally, each expansion cohort will include 24 response-evaluable subjects who are naïve to anti-PD-1/anti-PD-L1 therapy and 6 response-evaluable subjects who are relapsed/refractory to prior anti-PD-1/anti-PD-L1 therapy. Ten response-evaluable subjects in each expansion cohort will first receive single-agent treatment with Compound A for 2 weeks at the RP2D previously determined in combination with nivolumab. Following the 2-week, single-agent treatment, Compound A treatment will continue (at the same dose) in combination with nivolumab during Week 3 and beyond.

The subset of expansion subjects who will be treated with single-agent Compound A at its combination RP2D during Weeks 1 and 2 should have accessible tumors for core or excisional biopsy and provide permission for the biopsies to be taken. These subjects will undergo mandatory biopsies before single-agent Compound A treatment begins, at the end of the 2-week treatment window, and after 6 weeks of treatment with Compound A in combination with nivolumab; an optional biopsy will also be taken at the time of PD. The biopsies will be used for biomarker analysis evaluating the effect of Compound A on tumor cells and on immune/stromal cells supporting tumor tissue.

The remaining 20 response-evaluable subjects in each expansion cohort will receive Compound A at its RP2D in combination with nivolumab, starting from Week 1, Day 1.

During dose escalation, serial blood samples for assessment of Compound A plasma PK will be collected for 24 hours after Compound A dosing on Cycle 1 Days 1 and 15, the days on which both Compound A and nivolumab are administered. Although the risk of drug-drug interaction between Compound A and nivolumab is predicted to be low, Compound A plasma PK following combination administration in the dose escalation will be compared with historical plasma PK following single-agent administration to confirm no clinically meaningful differences in Compound A PK between the single-agent and combination settings. For purposes of population PK analysis, sparse collection of PK samples will occur in the expansion cohorts during both the single-agent and combination administration periods.

All subjects in the expansion cohorts will be treated until either PD or occurrence of unacceptable toxicities. The objectives of these expansion cohorts are to evaluate efficacy of Compound A in combination with nivolumab as measured by overall response rate (ORR) and to determine the safety and tolerability of Compound A in combination with nivolumab.

Primary Objectives: (1) To determine the MTD/RP2D of Compound A when administered in combination with nivolumab (dose escalation). (2) To determine the efficacy of Compound A plus nivolumab as measured by ORR (dose expansion).

Secondary Objectives: (1) To determine the safety and tolerability of Compound A when administered in combination with nivolumab. (2) To evaluate other efficacy measures such as disease control rate (response plus stable disease), duration of response (DOR), rate of PD at 6 months, progression-free survival (PFS), and overall survival (OS). (3) To characterize the plasma PK of Compound A when administered in combination with nivolumab.

### Subject Population.

Dose escalation: Subjects aged 18 years or older with advanced solid tumors for whom 1 or more prior lines of therapy have failed and who have no effective therapeutic options available based on investigator assessment. Dose expansion: Subjects aged 18 years or older with: (1) metastatic TNBC with ≥1 prior line of chemotherapy; (2) locally advanced or metastatic NSCLC that has progressed on or after a prior platinum-based chemotherapy; or (3) locally advanced or metastatic HNSCC that has progressed or recurred within 6 months of the last platinum-based chemotherapy.

### Number of Subjects:

It is expected that approximately 120 subjects will be enrolled in the study: approximately 9 to 12 subjects in the dose escalation cohort and approximately 36 subjects in each of the 3 dose expansion cohorts.

### Dose Level(s):

Compound A: oral daily dosing with 3+3 dose escalation planned at 60 and 100 mg. The RP2D determined in combination with nivolumab during dose escalation will be used for dose expansion cohorts. Nivolumab: 3 mg/kg IV dosing over 60 minutes every 2 weeks (Day 1 and Day 15 of each 28-day cycle). For subjects participating in the 2-week monotherapy run-in with Compound A, the first dose will be on Cycle 1 Day 15.

### Duration of Treatment.

Treatment will continue until disease progression (PD), unacceptable toxicities, or withdrawal due to other reasons. The estimated treatment duration is 12 months.

### Route of Administration:

Compound A: oral. Nivolumab: IV.

### Period of Evaluation:

PFS follow-up of 6 months (for subjects who discontinue due to reasons other than PD) and OS follow-up of 12 months from the last dose of study drug are planned.

### Arms:

Part 1 Compound A+Nivolumab: Compound A 60 mg, tablets, orally, once daily in each 28-day treatment cycles in combination with nivolumab 3 milligram per kilogram (mg/kg), infusion, intravenously over 60 minutes, on Days 1 and 15 in each 28 days treatment cycles until PD or unacceptable toxicity. For subjects who will receive 2 weeks of Compound A monotherapy before starting combination treatment, the first nivolumab infusion will be administered on Cycle 1 Day 15. Dose escalation of Compound A to 100 mg may be done using a 3+3 dose escalation design to determine a maximum tolerated dose (MTD) and/or recommended Phase 2 dose (RP2D).

Part 2 Metastatic TNBC: Subjects with metastatic triple-negative breast cancer (TNBC) will receive Compound A at RP2D as determined in Part 1, tablets, orally, once daily in each 28-day treatment cycles in combination with nivolumab 3 mg/kg, infusion over 60 minutes, intravenously, once only on Day 15 of Cycle 1 (following first 2 week monotherapy of Compound A) and thereafter Days 1 and 15 in each 28 days treatment cycles until progressive disease or unacceptable toxicity.

Part 2 Metastatic NSCLC: Subjects with metastatic NSCLC will receive Compound A at RP2D as determined in Part 1, tablets, orally, once daily in each 28-day treatment cycles in combination with nivolumab 3 mg/kg, infusion over 60 minutes, intravenously, once only on Day 15 of Cycle 1 (following first 2 week monotherapy of Compound A) and thereafter Days 1 and 15 in each 28 days treatment cycles until progressive disease or unacceptable toxicity.

Part 2 Metastatic HNSCC: Subjects with metastatic HNSCC will receive Compound A at RP2D as determined in Part 1, tablets, orally, once daily in each 28-day treatment cycles in combination with nivolumab 3 mg/kg, infusion over 60 minutes, intravenously, once only on Day 15 of Cycle 1 (following first 2 week monotherapy of Compound A and thereafter Days 1 and 15 in each 28 days treatment cycles until progressive disease or unacceptable toxicity.

### Criteria for Inclusion:

(1) Is a male or female subjects aged 18 years or older. (2) Has eastern Cooperative Oncology Group (ECOG) performance status 0 or 1.

(3) Female subjects who: (a) are postmenopausal for at least 1 year before the screening visit, or (b) are surgically sterile, or (c) if childbearing potential, agree to practice 2 effective methods of contraception, at the same time, from the time of signing the informed consent through 180 days after the last dose of study drug, or (d) agree to practice true abstinence, when this is in line with the preferred and usual lifestyle of the subjects. Periodic abstinence (e.g., calendar, ovulation, symptothermal, postovulation methods) and withdrawal are not acceptable methods of contraception. Male subjects, even if surgically sterilized (i.e., status postvasectomy), who: (a) agree to practice effective barrier contraception during the entire study treatment period and through 180 days after the last dose of study drug, or (b) agree to practice true abstinence, when this is in line with the preferred and usual lifestyle of the subjects. Periodic abstinence (e.g., calendar, ovulation, symptothermal, postovulation methods for the female partner) and withdrawal are not acceptable methods of contraception.

(4) Voluntary written consent must be given before performance of any study-related procedure not part of standard medical care, with the understanding that consent may be withdrawn by the subjects at any time without prejudice to future medical care. (5) Suitable venous access for the study-required blood sampling, including PK and pharmacodynamic sampling.

(6) Clinical laboratory values as specified below within 28 days before the first dose of study drug: (a) Total bilirubin must be less than equal to (<=) 1.5*the upper limit of normal (ULN). (b) Alanine aminotransferase (ALT) and aspartate aminotransferase (AST) must be <=2.5*ULN. (c) Serum creatinine must be <=1.5*ULN or creatinine clearance or calculated creatinine clearance must be greater than (>) 50 milliliter per minute (mL/minute). (d) Hemoglobin must be greater than equal to (>=) 8 gram per deciliter (g/dL), absolute neutrophil count (ANC) must be >=1500 per microliter (/mcL), and platelet count must be >=75,000/mcL.

(7) Recovered (i.e., <=Grade 1 toxicity) from the reversible effects of prior anticancer therapy. (8) To be enrolled in the dose escalation phase of the study, subjects must have a radiographically or clinically evaluable tumor, but measurable disease as defined by RECIST version 1.1 is not required for participation in this study. EISENHAUER et al., Eur. J. Cancer, 45(2):228-247 (2009).

(9) To be enrolled in the TNBC expansion cohort, subjects must have: (a) Histologically confirmed, metastatic TNBC with measurable disease per Response Evaluation Criteria in Solid Tumors (RECIST) version 1.1. (b) Triple-negative disease (estrogen receptor, progesterone receptor, and human epidermal growth factor receptor 2 (HER 2) negativity) confirmed on a histological biopsy of a metastatic tumor lesion (receptor conversion not allowed). (c) Safely accessible tumor lesions (based on investigator's assessment) for serial pretreatment and posttreatment biopsies are required for subjects receiving Compound A monotherapy run-in treatment for 2 weeks followed by Compound A plus nivolumab combination treatment (similar approximately 10/30 response-evaluable subjects); adequate, newly obtained, core or excisional biopsy of a metastatic tumor lesion not previously irradiated is required. Mandatory biopsies will be taken before Compound A monotherapy, after the 2 weeks of Compound A monotherapy, and after 6 weeks of Compound A plus nivolumab combination therapy. An optional biopsy may be taken at PD with additional consent from the subject. (d) One, two, or three line(s) of chemotherapy for metastatic disease and with progression of disease on last treatment regimen. For the purposes of this study, neoadjuvant and/or adjuvant chemotherapy regimens do not count as a prior line of therapy. Prior treatment must include an anthracycline and/or a taxane in the neoadjuvant, adjuvant, or metastatic setting with the exception for subjects who are clinically contraindicated for these chemotherapies.

(10) To be enrolled in the NSCLC expansion cohort, subjects must have: (a) Locally advanced or metastatic (stage IIIB, stage IV, or recurrent) NSCLC with measurable lesions per RECIST verion 1.1. (b) PD during or following at least 1 prior treatment. Subjects should have received a prior platinum-containing, 2-drug regimen for locally advanced, unresectable/inoperable or metastatic NSCLC had or disease recurrence within 6 months of treatment with a platinum-based adjuvant/neoadjuvant regimen or combined modality (e.g., chemoradiation) regimen with curative intent. (c) Subjects with epidermal growth factor receptor (EGFR) or anaplastic lymphoma kinase (ALK) genomic alternations should have PD on prior therapy for these aberrations. (d) Safely accessible tumor lesions (based on investigator's assessment) for serial pretreatment and posttreatment biopsies are required for subjects receiving Compound A monotherapy run-in treatment for 2 weeks followed by Compound A plus nivolumab combination treatment (approximately 10/30 response-evaluable subjects); adequate, newly obtained, core or excisional biopsy of a metastatic tumor lesion not previously irradiated is required. Mandatory biopsies will be taken before Compound A monotherapy, after the 2 weeks of Compound A monotherapy, and after 6 weeks of Compound A plus nivolumab combination therapy. An optional biopsy may be taken at PD with additional consent from the subject.

(11) To be enrolled in the HNSCC expansion cohort, subjects must have: (a) Histologically confirmed recurrent or metastatic HNSCC (oral cavity, pharynx, larynx), stage IIIIV, and not amenable to local therapy with curative intent (surgery or radiation therapy with or without chemotherapy). Histologically confirmed recurrent or metastatic squamous cell carcinoma of unknown primary or nonsquamous histologies (e.g., mucosal melanoma) are not allowed. Histologically confirmed recurrent or metastatic carcinoma of the nasopharynx is allowed, but these subjects will not be included as response-evaluable subjects for efficacy analysis of HNSCC. (b) Measurable disease per RECIST version 1.1. (c) Tumor progression or recurrence within 6 months of the last dose of platinum-based therapy in the adjuvant (i.e., with radiation after surgery), primary (i.e., with radiation), recurrent, or metastatic setting. (d) Safely accessible tumor lesions (based on investigator's assessment) for serial pretreatment and posttreatment biopsies are required for subjects receiving Compound A monotherapy run-in treatment for 2 weeks followed by Compound A plus nivolumab combination treatment (approximately 10/30 response-evaluable subjects); adequate, newly obtained, core or excisional biopsy of a metastatic tumor lesion not previously irradiated is required. Mandatory biopsies will be taken before Compound A monotherapy, after the 2 weeks of Compound A monotherapy, and after 6 weeks of Compound A plus nivolumab combination therapy. An optional biopsy may be taken at PD with additional consent from the subject.

### Exclusion Criteria:

(1) Has active brain metastases or leptomeningeal metastases.

(2) Has active, known, or suspected autoimmune disease.

(3) Diagnosis of immunodeficiency or any condition requiring systemic treatment with corticosteroids (>10 mg daily prednisone equivalents) or other immunosuppressive medications within 14 days of treatment.

(4) Has history of pneumonitis requiring treatment with steroids; history of idiopathic pulmonary fibrosis, drug-induced pneumonitis, organizing pneumonia, or evidence of active pneumonitis on screening chest computed tomography scan; history of radiation pneumonitis in the radiation field (fibrosis) is permitted.

(5) Has history of interstitial lung disease.

(6) Prior therapy with experimental antitumor vaccines; any T-cell co-stimulation agents or inhibitors of checkpoint pathways, such as anti-programmed cell death protein 1 (PD-1), anti-programmed cell death 1 ligand 1 (PD-L1), anti-programmed cell death 1 ligand 2 (PD-L2), anti-CD137, or anti-CTLA-4 antibody; or other agents specifically targeting T cells are prohibited. However, in each of the expansion cohorts, 6 response-evaluable subjects with prior exposure to anti-PD-1 or anti-PD-L1 agents will be allowed to enroll.

(7) Has any serious medical or psychiatric illness, including drug or alcohol abuse, that could, in the investigator's opinion, potentially interfere with the completion of treatment according to this protocol.

(8) Life-threatening illness unrelated to cancer.

(9) Is female subject who are lactating and breast-feeding or a positive serum pregnancy test during the Screening period or a positive urine pregnancy test on Day 1 before the first dose of study drug.

(10) Systemic anticancer treatment or radiotherapy less than 2 weeks before the first dose of study treatment (=<4 weeks for monoclonal antibodies with evidence of PD) or not recovered from acute toxic effects from prior chemotherapy and radiotherapy.

(11) Prior treatment with investigational agents=<21 days or =<5*their half-lives (whichever is shorter) before the first dose of study treatment. A minimum of 10 days should elapse from prior therapy to initiating protocol therapy.

(12) Major surgery within 14 days before the first dose of study drug and not recovered fully from any complications from surgery.

(13) Systemic infection requiring intravenous antibiotic therapy or other serious infection within 14 days before the first dose of study drug.

(14) Treatment with high-dose corticosteroids for anticancer purposes within 14 days before the first dose of Compound A; daily dose equivalent to 10 mg oral prednisone or less is permitted. Corticosteroids for topical use or in nasal spray or inhalers are allowed.

(15) Known human immunodeficiency virus (HIV) positive (testing not required).

(16) Known hepatitis B surface antigen-positive or known or suspected active hepatitis C infection (testing not required).

(17) Active secondary malignancy that requires treatment. Subjects with nonmelanoma skin cancer or carcinoma in situ of any type are not excluded if they have undergone complete resection and are considered disease-free at the time of study entry.

(18) Any clinically significant co-morbidities, such as uncontrolled pulmonary disease, known impaired cardiac function or clinically significant cardiac disease (specified below), active central nervous system disease, active infection, or any other condition that could compromise the subject's participation in the study. Subjects with any of the following cardiovascular conditions are excluded: (a) Acute myocardial infarction within 6 months before starting study drug. (b) Current or history of New York Heart Association Class III or IV heart failure. (c) Evidence of current uncontrolled cardiovascular conditions including cardiac arrhythmias, angina, pulmonary hypertension, or electrocardiographic evidence of acute ischemia or active conduction system abnormalities. (d) Friderichia corrected QT interval (QTcF)>450 milliseconds (msec) (men) or >475 msec (women) on a 12-lead electrocardiogram (ECG) during the Screening period. (e) Abnormalities on 12-lead ECG including, but not limited to, changes in rhythm and intervals that in the opinion of the investigator are considered to be clinically significant.

(19) Known gastrointestinal (GI) disease or GI procedure that could interfere with the oral absorption or tolerance of Compound A including difficulty swallowing tablets; diarrhea >Grade 1 despite supportive therapy.

(20) Use or consumption of any of the following substances: (a) Medications or supplements that are known to be inhibitors of P-glycoprotein (P-gp) and/or strong reversible inhibitors of cytochrome P450 (CYP)3A within 5 times the inhibitor half-life (if a reasonable half-life estimate is known) or within 7 days (if a reasonable half-life estimate is unknown) before the first dose of study drug. Use of inhibitors of P-glycoprotein (P-gp) and/or strong reversible inhibitors of cytochrome P450 (CYP)3A, such as amiodarone, azithromycin, captopril, carvedilol, cyclosporine, diltiazem, dronedarone, erythromycin, felodipine, itraconazole, ketoconazole, nefazodone, posaconazole, quercetin, quinidine, ranolazine, ticagrelor, verapamil, and voriconazole is not permitted during the study. The list of prohibited strong cytochrome P450 (CYP) 3A reversible inhibitors and/or P-gp inhibitors is not exhaustive and is based on the US FDA draft DDI guidance. (b) Medications or supplements that are known to be strong CYP3A mechanism-based inhibitors, such as clarithromycin, conivaptan, mibefradil, telithromycin, or strong CYP3A inducers and/or P-gp inducers, such as avasimibe, carbamazepine, phenobarbital, phenytoin, primidone, rifabutin, rifapentine, rifampin, St John's wort, within 7 days, or within 5 times the inhibitor or inducer half-life (whichever is longer), before the first dose of study drug. The use of these agents is not permitted during the study. The list of prohibited strong CYP3A inducers and/or P-gp inducers is not exhaustive and is based on the US FDA draft DDI guidance. Grapefruit-containing foods or beverages within 5 days before the first dose of study drug. (c) Grapefruit-containing food or beverages within 5 days before the first dose of study drug. Note that grapefruit-containing food and beverages are prohibited during the study.

(21) For dose expansion subjects who will have tumor biopsies collected: (a) ECOG performance status >1. (b) Activated partial thromboplastin time (aPTT) or plasma thromboplastin (PT) outside the normal range. (c) Platelet count <75,000/mcL. (d) Known bleeding diathesis or history of abnormal bleeding, or any other known coagulation abnormalities that would contraindicate the tumor biopsy procedure. (e) Ongoing therapy with any anticoagulant or antiplatelet agents (eg, aspirin, clopidogrel, coumadin, heparin, or warfarin) that cannot be held to permit tumor biopsy.

### Main Criteria for Evaluation and Analyses.

Primary endpoints: (1) MTD or RP2D (dose escalation). (2) ORR as assessed by the investigator per RECIST version 1.1 (dose expansion).

Secondary endpoints: (1) Percentage of subjects with adverse events (AEs), Grade 3 and Grade 4 AEs, serious AEs, and discontinuations for AEs, and clinical laboratory values and vital sign measurements outside the normal range that are of clinical significance. (2) Disease control rate. (3) DOR. (4) Rate of PD at 6 months. (5) PFS. (6) OS. (7) Summary statistics of Compound A maximum (peak) plasma concentration, first time to reach maximum (peak) plasma concentration, and area under the plasma concentration versus time curve over the dosing interval on Cycle 1, Days 1 and 15, by dose escalation cohort.

Statistical considerations: The MTD/MAD will be estimated by a standard 3+3 method using data collected in the dose escalation phase. AEs will be summarized by treatment group and overall. Categorical variables such as ORR, disease control rate, and rate of PD at 6 months will be tabulated by treatment group and overall. Time to event variables such as DOR, PFS, and OS will be analyzed using Kaplan-Meier survival curves, and Kaplan-Meier medians (if estimable) will be provided. PK parameters will be summarized as appropriate.

Sample size justification: During the dose escalation phase, dose escalation will be conducted according to a standard 3+3 dose escalation schema, and approximately 9 to 12 dose-limiting toxicity-evaluable subjects will be enrolled. The MTD/RP2D cohort will have at least 6 subjects. The sample sizes for each expansion cohort are estimated using a 1-sided exact binomial test at a significance level of α=0.1 with a power of 80%. Each cohort uses a null hypothesis of response rate ≤20%, versus an alternative hypothesis of response rate ≥40% for subjects who are naïve to anti-PD/PD-L1 and any other immune-directed antitumor therapies. Therefore, approximately 24 response-evaluable subjects for each cohort will be needed. In addition, 6 response-evaluable subjects with prior exposure to a PD-1 or PD-L1 inhibitor will be enrolled in each expansion cohort. In total, 30 response-evaluable subjects for each cohort and 90 response-evaluable subjects in total (˜108 subjects based on a 15% drop-out rate) will be n eeded for all expansion cohorts.

### Primary Outcome Measure:

(1) Part 1, MTD (baseline up to Day 28): MTD: highest dose level for which less than or equal to (=<)1 of 6 subjects in a dose cohort experience. Part 1, RP2D (baseline up to 6 months): the RP2D of Compound A will be determined in Part 1 (dose escalation) on the basis of the safety, tolerability, preliminary pharmacokinetics (PK), and efficacy data observed in Cycle 1 and beyond.

(2) Part 2, ORR (baseline up to 6 months after the last dose of study treatment, approximately 18 months): ORR is defined as the percentage of subjects with complete response (CR), or partial response (PR) according to Response Evaluation Criteria in Solid Tumors (RECIST) version 1.1. CR is defined as complete disappearance of all target lesions and non-target disease, with the exception of nodal disease. All nodes, both target and non-target, must decrease to normal (short axis <10 mm). No new lesions. PR is defined as >=30% decrease under baseline of the sum of diameters of all target lesions. The short axis is used in the sum for target nodes, while the longest diameter is used in the sum for all other target lesions. No unequivocal progression of non-target disease. No new lesions.

### Secondary Outcome Measures.

(1) Percentage of subjects experiencing 1 or more treatment-emergent adverse events (TEAEs) (baseline through 28 days after the last dose of study drug or to the start of subsequent alternative anticancer therapy, whichever occurs first (approximately up to 12 months)).

(2) Percentage of subjects with 1 or more grade 3 and grade 4 AEs (baseline through 28 days after the last dose of study drug or to the start of subsequent alternative anticancer therapy, whichever occurs first (approximately up to 12 months)).

(3) Percentage of subjects experiencing serious adverse events (SAES) (baseline through 28 days after the last dose of study drug or to the start of subsequent alternative anticancer therapy, whichever occurs first (approximately up to 12 months)).

(4) Percentage of subjects with TEAEs resulting in study drug discontinuation (baseline through 28 days after the last dose of study drug or to the start of subsequent alternative anticancer therapy, whichever occurs first (approximately up to 12 months)).

(5) Number of subjects with clinically significant laboratory values (baseline through 28 days after the last dose of study drug or to the start of subsequent alternative anticancer therapy, whichever occurs first (approximately up to 12 months)).

(6) Number of subjects with clinically significant vital sign measurements (baseline through 28 days after the last dose of study drug or to the start of subsequent alternative anticancer therapy, whichever occurs first (approximately up to 12 months)).

(7) Part 2: percentage of subjects with disease control (baseline up to 6 months after the last dose of study treatment (approximately 18 months)). Disease control rate: percentage of subjects with CR, PR or stable disease (SD) according to RECIST version 1.1. CR: complete disappearance of all target lesions and non-target disease, with exception of nodal disease.

(8) Part 2: Duration of Response (DOR) (from first dose until discontinuation of study drug due to disease progression, unacceptable toxicity, or death (approximately 18 months)). DOR is defined as the time from the date of first documentation of a response to the date of first documentation of PD according to RECIST version 1.1 criteria. PD is defined as >=25% increase from lowest value in: serum/urine M-component; difference between involved, uninvolved FLC levels; bone marrow plasma cell percent; development of new bone lesions or soft tissue plasmacytomas development or increase in the size of existing bone lesions or soft tissue plasmacytomas; hypercalcaemia development. Subjects without documentation of PD at the time of analysis will be censored at the date of their last response assessment that is SD or better.

(9) Part 2: percentage of subjects with progression of disease (PD) at Month 6. PD is defined as >=25% increase from lowest value in: serum/urine M-component; difference between involved, uninvolved FLC levels; bone marrow plasma cell percent; development of new bone lesions or soft tissue plasmacytomas development or increase in the size of existing bone lesions or soft tissue plasmacytomas; hypercalcaemia development.

(10) Part 2: progression free survival (PFS) (baseline up to 6 months after the last dose of study treatment (approximately 18 months)). Progression-free survival is defined as the time from the date of randomization to the date of first documentation of progressive disease or death due to any cause, whichever occurs first. PD is defined as >=25% increase from lowest value in: serum/urine M-component; difference between involved, uninvolved FLC levels; bone marrow plasma cell percent; development of new bone lesions or soft tissue plasmacytomas development or increase in the size of existing bone lesions or soft tissue plasmacytomas; hypercalcaemia development.

(11) Part 2: overall survival (OS) (baseline up to 6 months after the last dose of study treatment (approximately 18 months)). Overall survival is defined as the time from study entry to the time of death.

(12) Part 1: maximum observed plasma concentration (Cmax) for Compound A (Cycle 1: Days 1 and 15: predose and at multiple time points (up to 8 hours)).

(13) Part 1: time to reach the Cmax (Tmax) for Compound A (Cycle 1: Days 1 and 15: predose and at multiple time points (up to 8 hours)).

(14) Part 1: area under the plasma concentration-time curve from time 0 to time tau (AUC tau)(Cycle 1: Days 1 and 15: predose and at multiple time points (up to 8 hours)).

### Example 17

Clinical study design—a study of Compound A in combination with venetoclax in subjects with advanced non-Hodgkin's lymphoma.

Data from nonclinical studies support the potential for Compound A to be an effective agent in treating subjects in combination with venetoclax in relapsed or refractory NHL.

The results of a clinical study of Compound A in combination with venetoclax in subjects with advanced NHL can inform about various parameters (e.g., MTD and RP2D) for administration of Compound A in combination with venetoclax in subjects with other cancers. For example, such clinical study can be used to determine parameters (e.g., MTD and RP2D) for administration of Compound A in combination with venetoclax in subjects with advanced solid tumors such as TNBC, NSCLC, and HNSCC.

This study will look at the determination of MTD/RP2D and efficacy measured by overall response rate (ORR) in subjects who take Compound A in combination with venetoclax. The study will include a dose escalation phase (Part 1) and a dose expansion phase (Part 2).

This is a phase 1b dose escalation study of Compound A in combination with venetoclax in adult subjects with advanced NHL after at least 1 prior line of therapy. The primary objective of the study is to determine the maximum tolerated dose (MTD) and/or the recommended phase 2 dose (RP2D) of Compound A and venetoclax when administered in combination. Compound A and venetoclax doses will be escalated according to a Bayesian logistic regression model (BLRM) with overedose control escalation schema shown below. The Compound A/venetoclax MTD/RP2D will be determined from the collective experience in the clinic considering the safety data, preliminary pharmacokinetic (PK) data and any early anti-tumor activity observed along with the statistical inference from the BLRM.

During Cycle 1 the dose of venetoclax will be ramped up over a 3-week period for subjects who receive a maximum daily dose of 400 mg once daily (QD) and over a 4-week period for subjects who receive a maximum daily dose of 800 mg QD. In the ramp-up to the daily dose of 400 mg QD venetoclax, subjects will receive 100 mg QD venetoclax in Week 1, 200 mg QD venetoclax in Week 2, and 400 mg QD venetoclax in Week 3 and thereafter. In the ramp-up to the daily dose of 800 mg QD venetoclax, subjects will receive 100 mg QD venetoclax in Week 1, 200 mg QD venetoclax in Week 2, 400 mg QD venetoclax in Week 3, and 800 mg QD venetoclax in Week 4 and thereafter. The dose of Compound A (60 mg QD or 100 mg QD) will begin on Cycle one Day 1.

The starting dose of Compound A will be 60 mg QD and the starting dose of venetoclax (following ramp-up) will be 400 mg QD. Safety data collected from subjects enrolled at the starting dose will be added into BLRM to determine if more subjects should be enrolled at the starting dose, or if either of the doses should be escalated, and the optimal route for escalation. The design is adaptive and can accommodate intermediate doses.

Intermediate dose levels of Compound A between 60 and 100 mg in increments of 20 mg (e.g., 80 mg) or dose levels below the starting dose of 60 mg (e.g., 40 mg) may be also evaluated if appropriate. Each time only one of the two agent's dose can be escalated. For each cohort there should be at least 3 dose-limiting toxicity (DLT)-evaluable subjects. Compound A+venetoclax dose escalation will continue until the combination MTD is reached, or until 100 mg QD of Compound A (the maximally administered dose [MAD])+800 mg venetoclax is determined to be safe and tolerable, or until an RP2D, if different from the MTD or MAD, has been identified on the basis of the safety, tolerability, and preliminary PK and efficacy data (if available) observed in Cycle 1 and beyond. Alternative regimens/schedule are permissible, if such measures are needed for subject safety or for a better understanding of the dose-toxicity and dose-exposure relationship of Compound A or venetoclax.

Following dose escalation, the safety and tolerability of the MTD/RP2D of the Compound A+venetoclax combination will be further explored in 2 dose safety expansion cohorts, Cohort 1 in subjects with diffuse large B-cell lymphoma (DLBCL) and Cohort 2 in subjects with follicular lymphoma (FL).

Serial PK samples will be collected at pre-specified time points in Cycle 1 during dose escalation and safety expansion cohorts to characterize the PK of Compound A and venetoclax when administered in combination. Toxicity will be evaluated according to National Cancer Institute Common Terminology Criteria for Adverse Events version 4.03. Common Terminology Criteria for Adverse Events (CTCAE). National Cancer Institute, National Institutes of Health, U.S. Department of Health and Human Services Series v4.03. Jun. 14, 2010. Publication No. 09-5410.

Primary Objectives: (1) To determine the MTD/RP2D of Compound A when administered in combination with venetoclax. (2) To evaluate safety and tolerability of Compound A in combination with venetoclax.

Secondary Objectives: (1) To characterize the plasma PK of Compound A when administered in combination with venetoclax. (2) To observe preliminary efficacy of Compound A and venetoclax in combination in subjects with advanced non-Hodgkin lymphoma relapsed and/or refractory after ≥1 prior line of therapy.

### Subject Population:

Male and female subjects 18 years or older with histologically or cytologically confirmed diagnosis of advanced NHL of any histology (with the exception of subjects with Waldenstrom macroglobulinemia [WM], mantle cell lymphoma [MCL], chronic lymphocytic leukemia [CLL], post-transplant lymphoproliferative disease (PTLD), Burkitt's lymphoma, Burkitt-like lymphoma, or lymphoblastic lymphoma/leukemia), including radiographically or clinically measurable disease with ≥1 target lesion per International Working Group (IWG) criteria for malignant lymphoma CHESON B D et al., J. Clin. Oncol., 25(5):579-86(2007). Subjects must be refractory or relapsed after at least 1 prior line of therapy for whom no effective standard therapy is available per investigator's assessment and either (1) treatment naïve to; (2) relapsed/refractory to; or (3) treatment failure (due to other reasons) with ibrutinib, idelalisib, or any other investigational B cell receptor (BCR) pathway inhibitors not directly targeting spleen tyrosine kinase (SYK). Subjects must have Eastern Cooperative Oncology Group (ECOG) performance status score of 0 or 1, adequate organ and coagulation function, and life expectancy of greater than 3 months.

### Number of Subjects:

Estimated total of 50, including 18 dose-limiting toxicity (DLT)-evaluable in the dose escalation phase and 12 response-evaluable in each of the DLBCL and FL expansion cohorts, assuming a 15% dropout rate.

### Dose Level(s):

Compound A: planned 60 or 100 mg orally (PO), QD, plus one of the following: (1) Venetoclax: 100 mg QD in Week 1, 200 mg QD in Week 2, 400 mg QD in Week 3 and thereafter; or (2) 100 mg QD in Week 1, 200 mg QD in Week 2, 400 mg QD in Week 3, and 800 mg QD in Week 4 and thereafter. Compound A and venetoclax will be administered in 28 day cycles.

### Duration of Treatment:

Treatment will continue until disease progression, unacceptable toxicities, or withdrawal for other reasons. The estimated median treatment duration is 6 months.

### Route of Administration:

Compound A: oral. Venetoclax: oral.

### Period of Evaluation:

Subjects who discontinue the study for any reason other than death will continue to be followed for adverse events (AEs) for 28 days after the last administration of Compound A or until the start of subsequent anticancer therapy, whichever occurs first.

### Criteria for Inclusion.

(1) Is a male or female subjects 18 years or older.

(2) Subject must have histologically or cytologically confirmed diagnosis of advanced NHL of any histology (with the exception of subjects with WM, MCL, or CLL, PTLD, Burkitt's lymphoma, Burkitt-like lymphoma, or lymphoblastic lymphoma/leukemia).

(3) Subject must have advanced NHL which is refractory or relapsed after at least 1 prior line of therapy for whom no effective standard therapy is available per investigator's assessment. The treatment is (a) naïve to; (b) relapsed/refractory to; or (c) treatment failure (due to other reasons) with ibrutinib, idelalisib, or any other investigational BCR pathway inhibitors not directly targeting SYK.

(4) Has eastern Cooperative Oncology Group (ECOG) performance status 0 or 1.

(5) Subject must have adequate organ and coagulation function, including the following: (a) Bone marrow reserve consistent with: absolute neutrophil count (ANC)≥1,000/μL, platelet count ≥75,000/μL (≥50,000/μL for subjects with bone marrow involvement), and hemoglobin ≥8 g/dL (red blood cell [RBC] transfusion allowed ≥14 days before assessment). (b) Hepatic: total bilirubin ≤1.5× the upper limit of normal (ULN); alanine aminotransferase (ALT) and aspartate aminotransferase (AST)≤2.5×ULN. (c) Renal: creatinine clearance ≥60 mL/min as estimated by the Cockcroft-Gault equation or based on urine collection (12 or 24 hours). (d) a PTT and PT not to exceed 1.2×ULN. (e) Others: (i) Lipase ≤1.5×ULN and amylase ≤1.5×ULN with no clinical symptoms suggestive of pancreatitis or cholecystitis. (ii) Blood pressure ≤Grade 1 (hypertensive subjects are permitted if their blood pressure is controlled to ≤Grade 1 by hypertensive medications and glycosylated hemoglobin is ≤6.5%).

(6) Female subjects who: (a) are postmenopausal for at least 1 year before the screening visit; or (b) are surgically sterile; or (c) if childbearing potential, agree to practice 2 effective methods of contraception, at the same time, from the time of signing the informed consent through 180 days after the last dose of study drug; or (d) agree to practice true abstinence, when this is in line with the preferred and usual lifestyle of the subjects. Periodic abstinence (e.g., calendar, ovulation, symptothermal, postovulation methods) and withdrawal are not acceptable methods of contraception. Male subjects, even if surgically sterilized (i.e., status postvasectomy), who: (a) agree to practice effective barrier contraception during the entire study treatment period and through 120 days (or if the drug has a very long half-life, for 90 days plus five half-lives) after the last dose of study drug; or (b) agree to practice true abstinence, when this is in line with the preferred and usual lifestyle of the subjects. Periodic abstinence (e.g., calendar, ovulation, symptothermal, postovulation methods for the female partner) and withdrawal are not acceptable methods of contraception.

(7) Voluntary written consent must be given before performance of any study-related procedure not part of standard medical care, with the understanding that consent may be withdrawn by the subjects at any time without prejudice to future medical care. (8) Suitable venous access for the study-required blood sampling, including PK and pharmacodynamic sampling. (9) Recovered (i.e., ≤Grade 1 toxicity) from the reversible effects of prior anticancer therapy.

### Exclusion Criteria.

(1) Is a female subject who is lactating and breastfeeding or has a positive serum pregnancy test during the screening period or a positive urine pregnancy test on Day 1 before first dose of study drug.

(2) Subject with a central nervous system (CNS) lymphoma; active brain or leptomeningeal metastases, as indicated by positive cytology from lumbar puncture or computed tomography (CT) scan/magnetic resonance imaging (MRI).

(3) Has history of drug-induced pneumonitis requiring treatment with steroids; history of idiopathic pulmonary fibrosis, organizing pneumonia; or evidence of active pneumonitis on screening chest CT scan. History of radiation pneumonitis in the radiation field (fibrosis) is permitted.

(4) Subject requires the use of warfarin.

(5) Recent receipt of live attenuated vaccines.

(6) History of a prior significant toxicity, other than thrombocytopenia, from another B cell lymphoma (Bcl)-2 family protein inhibitor.

(7) Any serious medical or psychiatric illness that could, in the investigator's opinion, potentially interfere with the completion of treatment according to this protocol.

(8) Systemic anticancer treatment (including investigational agents) or radiotherapy <2 weeks before the first dose of study treatment (≤4 weeks for antibody-based therapy including unconjugated antibody, antibody-drug conjugate, and bi-specific T-cell engager agents ≤8 weeks for cell-based therapy or antitumor vaccine) or have not recovered from acute toxic effects from prior chemotherapy and radiotherapy.

(9) Major surgery within 14 days before the first dose of study drug and not recovered fully from any complications from surgery.

(10) Systemic infection requiring intravenous antibiotic therapy or other serious infection within 14 days before the first dose of study drug.

(11) Known human immunodeficiency virus (HIV) positive.

(12) Known hepatitis B surface antigen-positive, or known or suspected active hepatitis C infection.

(13) Subjects with another malignancy within 2 years of study start. Subjects with nonmelanoma skin cancer or carcinoma in situ of any type are not excluded if they have undergone complete resection and are considered disease-free at the time of study entry.

(14) Any clinically significant co-morbidities, such as uncontrolled pulmonary disease, known impaired cardiac function or clinically significant cardiac disease (specified below), active central nervous system disease, active infection, or any other condition that could compromise the subject's participation in the study. Subjects with any of the following cardiovascular conditions are excluded: (a) Acute myocardial infarction within 6 months before starting study drug. (b) Current or history of New York Heart Association Class III or IV heart failure. (c) Evidence of current uncontrolled cardiovascular conditions including cardiac arrhythmias, angina, pulmonary hypertension, or electrocardiographic evidence of acute ischemia or active conduction system abnormalities. (d) Friderichia corrected QT interval (QTcF)>450 milliseconds (msec) (men) or >475 msec (women) on a 12-lead electrocardiogram (ECG) during the Screening period. (e) Abnormalities on 12-lead ECG including, but not limited to, changes in rhythm and intervals that in the opinion of the investigator are considered to be clinically significant.

(15) Known gastrointestinal (GI) disease or GI procedure that could interfere with the oral absorption or tolerance of study drug, including difficulty swallowing capsules.

(16) Use or consumption of any of the following substances: (a) Medications or supplements that are known to be inhibitors of P-glycoprotein (P-gp), such as amiodarone, azithromycin, captopril, carvedilol, clarithromycin, conivaptan, cyclosporine, diltiazem, dronedarone, erythromycin, felodipine, itraconazole, ketoconazole, quercetin, quinidine, ranolazine, ticagrelor, verapamil, and/or strong or moderate reversible inhibitors of cytochrome P450 (CYP)3A, such as clarithromycin, conivaptan, itraconazole, ketoconazole, mibefradil, nefazodone, posaconazole, telithromycin, voriconazole, aprepitant, ciprofloxacin, diltiazem, erythromycin, fluconazole, verapamil, within 5 times the inhibitor half-life (if a reasonable half-life estimate is known) or within 7 days (if a reasonable half-life estimate is unknown) before the first dose of study drug. In general the use of these agents is not permitted during the study. The list of prohibited strong cytochrome P450 (CYP) 3A reversible inhibitors and/or P-gp inhibitors is not exhaustive and is based on the US FDA draft DDI guidance. (b) Medications or supplements that are known to be strong or moderate CYP3A mechanism-based inhibitors or strong CYP3A inducers, such as avasimibe, carbamazepine, phenytoin, rifampin, St. John's wort, and/or P-gp inducers such as avasimibe, carbamazepine, phenytoin, rifampin, St John's wort, within 7 days, or within 5 times the inhibitor or inducer half-life (whichever is longer), before the first dose of study drug. in general the use of these agents is not permitted during the study. The list of prohibited strong CYP3A inducers and/or P-gp inducers is not exhaustive and is based on the US FDA draft DDI guidance. Grapefruit-containing food or beverages within 5 days before the first dose of study drug. (c) Food or beverages containing grapefruit within 5 days before the first dose of study drug. Note that food and beverages containing grapefruit, Seville orange, or Star fruit are not permitted during the study.

### Main Criteria for Evaluation and Analyses.

Primary endpoints: (1) Percentage of subjects with adverse events (AEs). (2) Percentage of subjects with Grade 3 adverse events (AEs). (3) Percentage of subjects with serious adverse events (SAEs). (4) Percentage of subjects who discontinued due to adverse events (AEs). (5) Number of subjects with a dose-limiting toxicity (DLT). (6) Percentage of subjects with clinically significant laboratory values. (7) Percentage of subjects with clinically significant vital sign measurements.

Secondary endpoints: (1) Summary statistics of Compound A and venetoclax maximum observed plasma concentration (Cₘₐₓ) on Cycle 1, Day 1 and either Day 22 or Day 28 by combination dose regimen. (2) Summary statistics of Compound A and venetoclax time of first occurrence of Cₘₐₓ (Tₘₐₓ) on Cycle 1, Day1 and either Day 22 or Day 28 by combination dose regimen. (3) Summary statistics of Compound A area under the plasma concentration versus time curve over the dosing interval (AUCτ) on Cycle 1, Day 1 and either Day 22 or Day 28 by combination dose regimen. (4) Overall response rate (ORR). (5) Complete response rate (CRR). (6) Time to progression (TPP).

Statistical considerations: It is estimated that approximately 50 subjects will be enrolled in this study, with approximately 18 dose-limiting toxicity (DLT)-evaluable subjects in the dose escalation phase, 12 response evaluable subjects each in DLBCL and FL safety expansion cohorts, assuming a 15% dropout rates.

An adaptive BLRM that implements escalation with overdose control will be used in this study for purposes of dose escalation recommendations and estimation of the MTD. The 5-parameter model will be used and updated after each cohort of 3 subjects. For each dose level, the posterior probability of having DLT rates that fall into the following intervals will be estimated: (a) [0, 0.16): underdosing. (b) [0.16, 0.35): targeted toxicity. (c) [0.35, 1.00): excessive toxicity.

Data from prior single agent studies in NHL for Compound A and venetoclax will be used as prior information in the BLRM. During the dose escalation, only one of the two drugs will be escalated in each step, and the escalation of one particular drug cannot exceed 100% of the current dose. The selection of the next recommended dose will be determined along with other available information on safety, PK and pharmacodynamics.

Sample Size Justification: The study will use an adaptive design using BLRM with safety data evaluation and PK guidance. The design allows flexible cohort size. The total number of subjects in the dose escalation is dependent on the observed safety profile and PK guidance, which will determine the number of subjects per combination dose cohort, as well as the number of dose escalations required to achieve the MTD. It is anticipated that approximately 18 subjects will be enrolled in the dose escalation in up to 6 cohorts. In addition, another 24 subjects will be enrolled for safety expansion, with 12 subjects in each of DLBCL and FL safety expansion. Assuming a 15% drop-out rate, the total sample size for this study will be approximately 50.

The embodiments described herein are intended to be merely exemplary, and those skilled in the art will recognize, or will be able to ascertain using no more than routine experimentation, numerous equivalents of specific compounds, materials, and procedures. All such equivalents are considered to be within the scope of the disclosure.

All combinations of the embodiments disclosed herein are within the scope of the disclosure.

All of the patents, patent applications and publications referred to herein are incorporated herein in their entireties. Citation or identification of any reference in this application is not an admission that such reference is available as prior art to this application. The full scope of the disclosure is better understood with reference to the appended claims.

We claim:

1. A method of treating a non-Hodgkin lymphoma comprising administering to a subject having the non-Hodgkin lymphoma a therapeutically effective amount of a combination comprising:
a SYK inhibitor; and
a second therapeutic agent.

2. The method of claim 1, wherein the non-Hodgkin lymphoma is chronic lymphocytic leukemia, indolent non-Hodgkin lymphoma, mantle cell lymphoma, post-transplant lymphoproliferative disorder, or diffuse large B-cell lymphoma.

3. The method of any one of claim 1 or 2, wherein the non-Hodgkin lymphoma is diffuse large B-cell lymphoma.

4. The method of any one of claims 1 to 3, wherein the SYK inhibitor is a compound of Formula II: 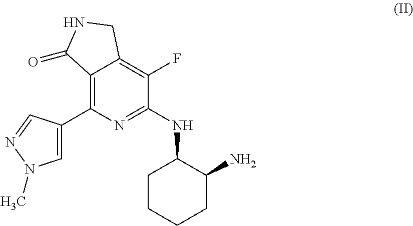 [SMILES: Cn1cc(-c2nc(N[C@@H]3CCCC[C@@H]3N)c(F)c3c2C(=O)NC3)cn1]
or a pharmaceutically acceptable salt thereof.

5. The method of any one of claims 1 to 4, wherein the SYK inhibitor is a compound of Formula III: 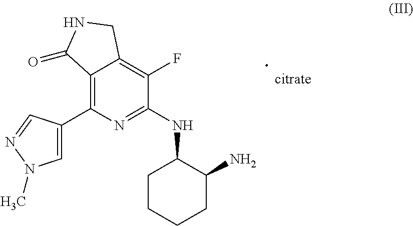 [SMILES: Cn1cc(-c2nc(N[C@@H]3CCCC[C@@H]3N)c(F)c3c2C(=O)NC3)cn1]
or a crystalline form thereof.

6. The method of any one of claims 1 to 5, wherein the combination further comprises one or more additional therapeutic agent(s).

7. A method of treating a non-Hodgkin lymphoma other than chronic lymphocytic leukemia comprising administering to a subject having the non-Hodgkin lymphoma a therapeutically effective amount of a combination comprising:
a SYK inhibitor; and
a second therapeutic agent.

8. The method of claim 7, wherein the non-Hodgkin lymphoma is indolent non-Hodgkin lymphoma, mantle cell lymphoma, post-transplant lymphoproliferative disorder, or diffuse large B-cell lymphoma.

9. The method of any one of claim 7 or 8, wherein the non-Hodgkin lymphoma is diffuse large B-cell lymphoma.

10. The method of any one of claims 7 to 9, wherein the SYK inhibitor is a compound of Formula II: 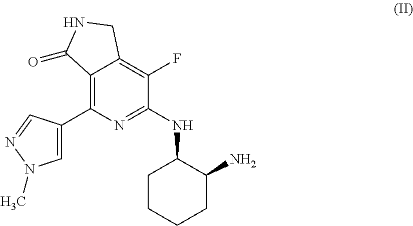 [SMILES: Cn1cc(-c2nc(N[C@@H]3CCCC[C@@H]3N)c(F)c3c2C(=O)NC3)cn1]
or a pharmaceutically acceptable salt thereof.

11. The method of any one of claims 7 to 10, wherein the SYK inhibitor is a compound of Formula III: 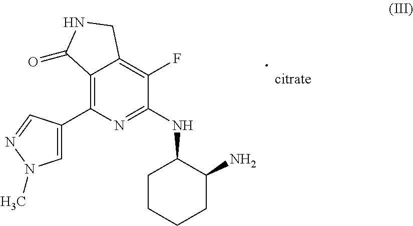 [SMILES: Cn1cc(-c2nc(N[C@@H]3CCCC[C@@H]3N)c(F)c3c2C(=O)NC3)cn1]
or a crystalline form thereof.

12. The method of any one of claims 7 to 11, wherein the combination further comprises one or more additional therapeutic agent(s).

13. A method of treating a non-Hodgkin lymphoma comprising administering to a subject having the non-Hodgkin lymphoma a therapeutically effective amount of a combination comprising:
a SYK inhibitor; and
a second therapeutic agent other than ibrutinib, idelalisib, or fludarabine.

14. The method of claim 13, wherein the non-Hodgkin lymphoma is chronic lymphocytic leukemia, indolent non-Hodgkin lymphoma, mantle cell lymphoma, post-transplant lymphoproliferative disorder, or diffuse large B-cell lymphoma.

15. The method of any one of claim 13 or 14, wherein the non-Hodgkin lymphoma is diffuse large B-cell lymphoma.

16. The method of any one of claims 13 to 15, wherein the SYK inhibitor is a compound of Formula II: 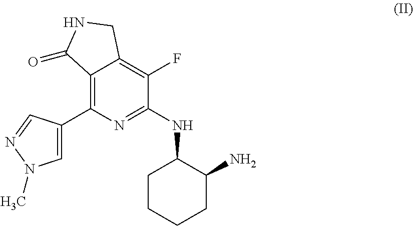 [SMILES: Cn1cc(-c2nc(N[C@@H]3CCCC[C@@H]3N)c(F)c3c2C(=O)NC3)cn1]
or a pharmaceutically acceptable salt thereof.

17. The method of any one of claims 13 to 16, wherein the SYK inhibitor is a compound of Formula III: 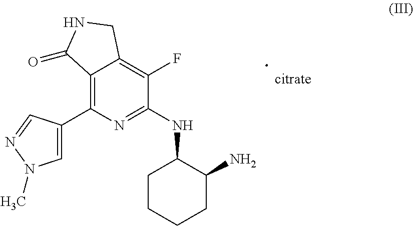 [SMILES: Cn1cc(-c2nc(N[C@@H]3CCCC[C@@H]3N)c(F)c3c2C(=O)NC3)cn1]
or a crystalline form thereof.

18. The method of any one of claims 13 to 17, wherein the combination further comprises one or more additional therapeutic agent(s).

19. The method of any one of claims 1 to 18, wherein the second therapeutic agent is a nitrogen mustard.

20. The method of claim 19, wherein the nitrogen mustard is selected from chlorambucil, uramustine, ifosfamide, melphalan, and bendamustine.

21. The method of claim 20, wherein the nitrogen mustard is bendamustine.

22. The method of any one of claims 19 to 21, wherein the combination further comprises an anti-CD20 antibody.

23. The method of claim 22, wherein the anti-CD20 antibody is selected from rituximab, obinutuzumab, ibritumomab tiuxetan, and tositumomab.

24. The method of claim 23, wherein the anti-CD20 antibody is rituximab.

25. The method of claim 24, wherein the nitrogen mustard is bendamustine and the anti-CD20 antibody is rituximab.

26. The method of any one of claims 1 to 18, wherein the second therapeutic agent is a nucleoside analog.

27. The method of claim 26, wherein the nucleoside analog is selected from gemcitabine and 5-FU.

28. The method of claim 27, wherein the nucleoside analog is gemcitabine.

29. The method of any one of claims 1 to 18, wherein the second therapeutic agent is an immunomodulatory agent.

30. The method of claim 29, wherein the immunomodulatory agent is a thalidomide analogue.

31. The method of claim 30, wherein the thalidomide analogue is lenalidomide.

32. The method of any one of claims 1 to 18, wherein the second therapeutic agent is a BTK inhibitor.

33. The method of any one of claims 1 to 12, wherein the second therapeutic agent is ibrutinib.

34. The method of any one of claims 1 to 18, wherein the second therapeutic agent is a BCL-2 inhibitor.

35. The method of claim 34, wherein the BCL-2 inhibitor is venetoclax.

36. The method of any one of claims 1 to 25 wherein the second agent is bendamustine administered on days 1 and 2 of a 21-day cycle at about 90 mg/m² dose.

37. The method of claim 36, wherein the combination further comprises rituximab administered on day 1 of a 21-day cycle at about 375 mg/m² dose.

38. The method of any one of claims 1 to 18 or 26 to 28, wherein the second agent is gemcitabine administered on days 1 and 8 of a 21 day cycle at about 1000 mg/m² dose.

39. The method of any one of claims 1 to 18 or 29 to 31, wherein the second agent is lenalidomide administered once daily on days 1 to 21 of a 28 day cycle at about 25 mg dose.

40. The method of any one of claims 1 to 12 or 33, wherein the second agent is ibrutinib administered once daily each day of a 28 day cycle at about 560 mg dose.

41. The method of any one of claims 1 to 18, 34, or 35 wherein the second agent is venetoclax administered once daily at about 10 mg to about 400 mg dose.

42. The method of any one of claims 1 to 18, wherein the second agent is nivolumab administered once every two weeks on day 1 and 15 of a 28-day cycle at about 3 mg/kg dose.

43. The method of any one of claims 1 to 18, wherein the second agent is nivolumab administered once every two weeks on day 1 and 15 of a 28-day cycle at about 240 mg dose.

44. The method of any one of claims 1 to 43, wherein the SYK inhibitor is administered once daily.

45. The method any one of claims 1 to 44, wherein a dose of the SYK inhibitor is about 20 mg to about 200 mg per day.

46. The method of claim 45, wherein the dose of the SYK inhibitor is about 40 mg per day, and wherein the SYK inhibitor is administered once daily.

47. The method of claim 45, wherein the dose of the SYK inhibitor is about 60 mg per day, and wherein the SYK inhibitor is administered once daily.

48. The method of claim 45, wherein the dose of the SYK inhibitor is about 80 mg per day, and wherein the SYK inhibitor is administered once daily.

49. The method of claim 45, wherein the dose of the SYK inhibitor is about 100 mg per day, and wherein the SYK inhibitor is administered once daily.

50. The method of any one of claims 1 to 49, wherein the SYK inhibitor is administered orally.

51. The method of any one of claims 1 to 50, wherein the second therapeutic agent and the SYK inhibitor are administered simultaneously.

52. The method of any one of claims 1 to 50, wherein the second therapeutic agent and the SYK inhibitor are administered sequentially.

53. The method of claim 52, wherein the second therapeutic agent is administered prior to the SYK inhibitor.

54. The method of claim 52, wherein the SYK inhibitor is administered prior to the second therapeutic agent.
